(19) United States
(12) Patent Application Publication
(10) Pub. No.: US 2024/0308939 A1
(43) Pub. Date: Sep. 19, 2024

(54) BI-FUNCTIONALIZED DICYCLOPENTADIENE MONOMER AND POLYMER EMBODIMENTS, AND METHODS OF MAKING AND USING SAME

(71) Applicant: UVIC INDUSTRY PARTNERSHIPS INC., Victoria (CA)

(72) Inventors: Jeremy Wulff, Victoria, BC (CA); Benjamin Godwin, Victoria, BC (CA)

(73) Assignee: UVIC INDUSTRY PARTNERSHIPS INC., Victoria, BC (CA)

(21) Appl. No.: 18/578,571

(22) PCT Filed: Mar. 30, 2022
(86) PCT No.: PCT/IB2022/052969

§ 371 Date: Jan. 11, 2024

Related U.S. Application Data
(60) Provisional application No. 63/221,718, filed on Jul. 14, 2021.

(51) Int. Cl. C07C 13/36; C07C 49/623; C07C 49/693; C08F 4/80; C08F 32/08; C08J 3/24

(52) U.S. Cl. CPC C07C 13/36; C07C 49/623; C07C 49/693; C08F 4/80; C08F 32/08; C08J 3/247

(57) ABSTRACT

Disclosed herein are embodiments of a bi-functionalized dicyclopentadiene monomer and polymer embodiments formed therefrom. The monomer embodiments exhibit tunability and can be used to form thermally stable homopolymers, copolymers, and/or crosslinked polymers.

### CROSS REFERENCE TO RELATED APPLICATION

This application claims the benefit of and priority to the earlier filing date of U.S. Provisional application No. 63/221,718, filed on Jul. 14, 2021, the entirety of which is incorporated herein by reference.

### FIELD

The present disclosure relates to bi-functionalized dicyclopentadiene monomer and polymer embodiments, as well as method embodiments for making and using the same.

### BACKGROUND

Polydicyclopentadiene (PDCPD) is a crosslinked organic polymer produced by ring-opening metathesis polymerization (ROMP) from dicyclopentadiene. Crosslinked PDCPD is used in a wide variety of commercial applications, and has found particular commercial success as a material for the production of body panels for tractors, construction vehicles, heavy trucks, and busses. These applications leverage PDCPD's high impact resistance (at both high and low temperatures), high degree of chemical corrosion resistance, high heat deflection temperature, and high glass transition temperature. Moreover, the low density of crosslinked PDCPD (ca. 1.05 g/cm³), coupled with its high storage modulus and tensile strength, suggests the possibility of expanded applications in the aerospace and marine sectors, as well as in the manufacture of anti-ballistic armor. For example, the twin objectives of light weight and self-healing armor are not met by today's steel-based add-on vehicle armor which have areal densities of up to 54 kg/m² for STANAG Level 1 protection, and 177 kg/m² for Level 3. Moreover, steel has a miniscule elastic deformation range coupled with a large plastic deformation, meaning that steel-based armor has almost no ability to recover their shape following ballistic impact.

Several disadvantages, however, have so far limited the broader application of PDCPD. These include, but are not limited to, an unpleasant odor (due to the presence of entrapped dicyclopentadiene monomer within the polymer matrix), a complex chemical structure (due to the likely presence of several different types of chemical crosslinks within the bulk polymer), and a lack of recyclability (something that is common to almost all thermoset materials). Also, PDCPD also has a low surface energy when freshly prepared (making it hard to paint or apply adhesives without a separate oxidation step), and is not chemically tunable (due to the unfunctionalized monomer feedstock). Thus, whereas polymers of functionalized ethylene (e.g., propylene, styrene, acrylic acid, methyl acrylate, acrylonitrile, methyl methacrylate, vinylidene chloride, etc.) exhibit a broad range of very distinct and very useful material properties, no such variability can be readily obtained for PDCPD-based polymers.

There exists a need in the art for functionalized dicyclopentadiene-based compounds and polymers formed therefrom that do not exhibit the disadvantages associated with un-functionalized PDCPD.

### SUMMARY

Disclosed herein are embodiments of a compound having a structure according to Formula I

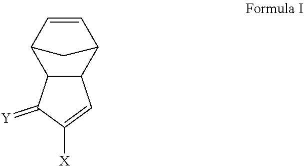

wherein
X is selected from hydrogen, deuterium, halogen, aliphatic, haloaliphatic, haloheteroaliphatic, heteroaliphatic, aromatic, an organic functional group, a detectable moiety, or an active component, or any combination of such groups; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, or an organic functional group; provided that if Y is O, then X is not hydrogen.

Also disclosed herein are embodiments of a polymer, comprising at least one monomer subunit having a structure according to Formula IA

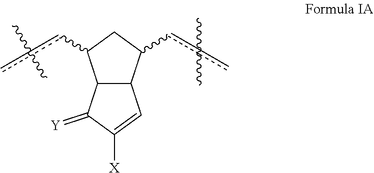

wherein
X is selected from hydrogen, deuterium, halogen, aliphatic, haloaliphatic, haloheteroaliphatic, heteroaliphatic, aromatic, an organic functional group, a detectable moiety, or an active component, or any combination thereof; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, an organic functional group, a detectable moiety, an active component, or a combination thereof.

Also disclosed herein are embodiments of a method, comprising:
forming a polymer by combining (i) a catalyst comprising ruthenium (Ru), molybdenum (Mo), tungsten (W), titanium (Ti), tantalum (Ta), or a combination thereof; and (ii) a compound having a Formula I

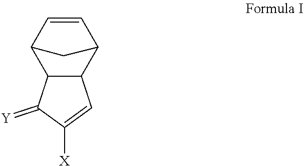

wherein
X is selected from hydrogen, deuterium, halogen, aliphatic, haloaliphatic, haloheteroaliphatic, heteroaliphatic, aromatic, an organic functional group, a detectable moiety, or an active component, or any combination of such groups; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, or an organic functional group.

The foregoing and other objects and features of the present disclosure will become more apparent from the following detailed description, which proceeds with reference to the accompanying figures.

### BRIEF DESCRIPTION OF THE DRAWINGS

FIG. 1 shows an infrared (IR) spectrum for endo-dicyclopentadienone.

FIG. 2 shows a comparison of 1H-NMR spectra (0-9 ppm) for four different polymer embodiments of the present disclosure.

FIG. 3 shows a comparison of ¹H NMR spectra (4.8-8.5 ppm) for four different polymer embodiments of the present disclosure.

FIG. 4 shows an IR spectrum for poly(endo-dicyclopentadienone) (also referred to herein as “oxaPDCPD”).

FIG. 5 shows an IR spectrum of progressively crosslinked oxaPDCPD.

FIG. 6 shows solid state ¹³C nuclear magnetic resonance (NMR) spectra of progressively crosslinked oxaPDCPD.

FIG. 7 shows variable temperature ¹H NMR spectra of oxaPDCPD.

FIG. 8 shows representative sequential DSC traces of oxaPDCPD.

FIG. 9 shows infrared (IR) spectra obtained from monitoring the thermal curing (also referred to herein as “crosslinking”) for a polymer embodiment of the present disclosure (namely, oxaPDCPD), along with comparison spectra for the monomer (endo and exo forms) and the linear polymer; the appearance of a new carbonyl stretch at longer wavenumbers is consistent with the formation of chemical crosslinks.

FIG. 10 shows IR spectra obtained from monitoring the thermal curing (also referred to herein as “crosslinking”) for a polymer embodiment of the present disclosure (namely, p, along with comparison spectra for the monomer (endo and exo forms); the appearance of a new carbonyl stretch at longer wavenumbers is consistent with the formation of chemical crosslinks.

FIG. 11 is a graph of percent weight (%) as a function of temperature (° C.) showing the thermal stability for crosslinked oxaPDCPD and poly(exo-dicyclopentadienone) using thermogravimetric analysis (TGA); the graph confirms that both polymers exhibit good thermal stability.

FIG. 12 shows photograph images of reaction-injection molding (RIM) samples of oxaPDCPD, prepared with various catalyst loadings; the darker colors correspond to higher concentrations of spent ruthenium catalyst.

FIG. 13 shows graphs comparing dynamic mechanical analysis (DMA) data for PDCPD (left) and oxaPDCPD (right), providing results for measured tan-delta (top), loss modulus (middle), and storage modulus (bottom).

FIG. 14 is a graph of stress-strain curves for low strain rate compression strength evaluations of PDCPD and oxaPDCPD.

FIG. 15 is a graph of stress-strain curves for high strain rate compression strength evaluations of PDCPD and oxaPDCPD.

FIG. 16 shows graphs of DMA data for reaction injection molded of a copolymer embodiment comprising PDCPCD and oxaPDCPD (referred to herein as “PDCPD-co-oxaPDCPD”), providing results for measured loss modulus (top), loss factor (middle), and storage modulus (bottom).

FIG. 17 is a graph showing stress-strain curves for ultimate tensile strength of PDCPD and oxaPDCPD.

FIG. 18 is a plot comparing simulated ballistic response for PDCPD to experimental results.

FIG. 19 is a plot comparing simulated ballistic response for oxaPDCPD-co-PDCPD to experimental results.

FIG. 20 is a plot comparing V50 (m/s) to areal density (kg/m²) for PDCPD, a 1:1 copolymer of oxaPDCPD-co-PDCPD (referred to as ‘coPDCPD’ in FIG. 20), and aluminum 5083.

FIG. 21 is a cutaway schematic representation of a composite-of-composite ballistic solution material (2100) comprising a strikeface fronting (2102), an polymer layer (2104) comprising a polymer embodiment according to the present disclosure, and a reinforcing fiber layer (2106) that provides additional strength.

### DETAILED DESCRIPTION

### I. Overview of Terms

The following explanations of terms and/or symbols are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

Certain functional group terms used herein include a symbol “-”, which is used to show how the defined functional group attaches to, or within, the compound to which it is bound. Also, a dashed bond (i.e., “ - - -”) as used in certain formulas described herein indicates an “optional” bond to a substituent or atom of the formula other than hydrogen in the sense that the bond (and in some embodiments, the substituent) may or may not be present. In any formulas comprising a dashed bond, if the optional bond and/or any corresponding substituent is not present, then the valency requirements of any atom(s) bound thereto is completed by a bond to a hydrogen atom.

The symbol “ [symbol] ” when not indicated as being directly attached to an atom of a formula

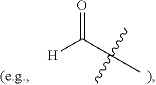 [SMILES: *C=O]

is used to indicate a bond disconnection in abbreviated structures/formulas provided herein. In some formulas, the symbol “ [symbol] ” is used and is directly bound to a carbon atom of a double bond

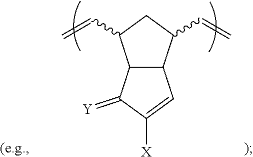

and, in these instances, this symbol indicates that the double bond can be a Z or E isomer unless otherwise specified. A person of ordinary skill in the art recognizes that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

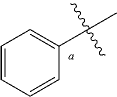 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃, or CH₃ wherein from 1 to 3 hydrogens are replaced by deuterium, such as in CDₓH₃₋ₓ.

As used herein, the term “substituted” refers to all subsequent modifiers in a term, for example in the term “substituted aliphatic-aromatic,” substitution may occur on the “aliphatic” portion, the “aromatic” portion or both portions of the aliphatic-aromatic group.

“Substituted,” when used to modify a specified group or moiety, means that at least one, and perhaps two or more, hydrogen atoms of the specified group or moiety is independently replaced with the same or different substituent groups. In a particular embodiment, a group, moiety, or substituent may be substituted or unsubstituted, unless expressly defined as either “unsubstituted” or “substituted.” Accordingly, any of the functional groups specified herein may be unsubstituted or substituted unless the context indicates otherwise or a particular structural formula precludes substitution. In particular embodiments, a substituent may or may not be expressly defined as substituted but is still contemplated to be optionally substituted. For example, an “aliphatic” or a “cyclic” moiety may be unsubstituted or substituted, but an “unsubstituted aliphatic” or an “unsubstituted cyclic” is not substituted. In one embodiment, a group that is substituted has at least one substituent up to the number of substituents possible for a particular moiety, such as 1 substituent, 2 substituents, 3 substituents, or 4 substituents.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

To facilitate review of the various examples of this disclosure, the following explanations of specific terms are provided:

Active Component: A molecule or fragment of a molecule which elicits a desirable response. In some embodiments, the response can be a biological response, a detectable response, a retardant or quenching response, or the like. In some embodiments, the active component can include, but is not limited to, antibacterial agents, antifungal agents, anticancer agents, peptides promoting cellular adhesion, signaling factors controlling cellular growth or motility, vitamins, cofactors, anticancer agents, fire retardants, sensors (e.g., pH sensors, H₂S sensors, antibody-based sensors, and the like), and combinations thereof.

Acyl Halide: —C(O)X, wherein X is a halogen, such as Br, F, I, or Cl.

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Aliphatic groups may be substituted with one or more groups other than hydrogen, such as aliphatic, heteroaliphatic, haloaliphatic, haloheteroaliphatic, aromatic, or an organic functional group.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z). Alkenyl groups may be substituted with one or more groups other than hydrogen, such as aliphatic, heteroaliphatic, haloaliphatic, haloheteroaliphatic, aromatic, or an organic functional group.

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds). Alkoxy groups may be substituted with one or more groups other than hydrogen, such as aliphatic, heteroaliphatic, haloaliphatic, haloheteroaliphatic, aromatic, or an organic functional group.

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl). Alkyl groups may be substituted with one or more groups other than hydrogen, such as aliphatic, heteroaliphatic, haloaliphatic, haloheteroaliphatic, aromatic, or an organic functional group.

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl). Alkenyl groups may be substituted with one or more groups other than hydrogen, such as aliphatic, heteroaliphatic, haloaliphatic, haloheteroaliphatic, aromatic, or an organic functional group.

Amide: —C(O)NR^{b}R^{c} or —NR^{b}C(O)R^{c} wherein each of R^{b} and R^{c} independently is selected from hydrogen, aliphatic, heteroaliphatic, haloaliphatic, haloheteroaliphatic, aromatic, or an organic functional group and can be substituted with one or more groups other than hydrogen, such as aliphatic, heteroaliphatic, haloaliphatic, haloheteroaliphatic, aromatic, or an organic functional group.

Amino (or Amine): —NR^{b}R^{c}, wherein each of R^{b} and R^{c} independently is selected from hydrogen, aliphatic, heteroaliphatic, haloaliphatic, haloheteroaliphatic, aromatic, or an organic functional group, and can be substituted with one or more groups other than hydrogen, such as aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

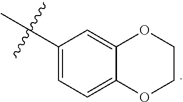 [SMILES: *c1ccc2c(c1)OCCO2]

However, in certain examples, context or express disclosure may indicate that the point of attachment is through a non-aromatic portion of the condensed ring system. For example,

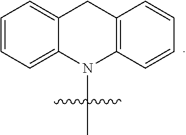 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: —O-aromatic. Aroxy groups may be substituted with one or more groups other than hydrogen, such as aliphatic, heteroaliphatic, haloaliphatic, haloheteroaliphatic, aromatic, or an organic functional group.

Azo: —N═NR^{a} wherein R^{a} is hydrogen, aliphatic, heteroaliphatic, haloaliphatic, haloheteroaliphatic, aromatic, or an organic functional group. Azo groups may be substituted with one or more groups other than hydrogen, such as aliphatic, heteroaliphatic, haloaliphatic, haloheteroaliphatic, aromatic, or an organic functional group.

Carbamate: —OC(O)NR^{b}R^{c}, wherein each of R^{b} and R^{c} independently is selected from hydrogen, aliphatic, heteroaliphatic, haloaliphatic, haloheteroaliphatic, aromatic, or an organic functional group. Carbamate groups can be substituted with one or more groups other than hydrogen, such as aliphatic, heteroaliphatic, haloaliphatic, haloheteroaliphatic, aromatic, or an organic functional group.

Carbonate: —OC(O)OR^{b}, wherein R^{b} is selected from aliphatic, heteroaliphatic, haloaliphatic, haloheteroaliphatic, aromatic, or an organic functional group. Carbonate groups can be substituted with one or more groups other than hydrogen, such as aliphatic, heteroaliphatic, haloaliphatic, haloheteroaliphatic, aromatic, or an organic functional group. In independent embodiments, R^{b} can be hydrogen.

Carboxyl: —C(O)OH.

Carboxylate: —C(O)O or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Chromogen: A compound that exhibits color and/or changes the color of substance, reaction mixture, or the like. Exemplary chromogens can include, but are not limited to, acridine dyes (including any derivatives of acridine), anthraquinone dyes (including any derivatives of anthraquinone), arylmethane dyes (including diarylmethane dyes, triarylmethane dyes), azo dyes (including dyes having an azo group), diazonium dyes (including diazonium salt compounds), nitro dyes (including dyes having a nitro group), nitroso dyes (including dyes having a nitroso functional group), phthalocyanine dyes (including derivatives of phthalocyanine), quinone-imine dyes (including azin dyes, indamins, indophenols, oxazins, oxazones, thiazines, or derivatives of quinone), thiazole dyes (including thiazole derivatives), safranin dyes (including derivatives of safranin), xanthene dyes (including derivatives of xanthene, fluorine, and rhodamine), and the like.

Copolymer: A polymeric material comprising two or more different types of repeating monomer subunits. The monomer subunits may occur randomly throughout the polymer, or may be grouped together into blocks of similar subunits. The monomer subunits can comprise monomers according to Formula I of the present disclosure; cyclic olefin-containing compounds, such as cyclobutene, cyclopentene, cycloheptene, cyclooctene, cyclononene, cyclodecene, cyclododecene, norbornene, cyclooctadiene, cyclononadiene, norbornadiene, and the like, as well as functionalized forms of such cyclic olefin-containing compounds; acyclic dienes; unfunctionalized dicyclopentadiene; or other types of monomer subunits, such as silyl ether-containing monomer subunits.

Cyano: —CN.

Disulfide: —SSR^{a}, wherein R^{a} is selected from hydrogen, aliphatic, heteroaliphatic, haloaliphatic, haloheteroaliphatic, aromatic, or an organic functional group. Disulfide groups can be substituted with one or more groups other than hydrogen, such as aliphatic, heteroaliphatic, haloaliphatic, haloheteroaliphatic, aromatic, or an organic functional group.

Dithiocarboxylic: —C(S)SR^{a} wherein R^{a} is selected from hydrogen, aliphatic, heteroaliphatic, haloaliphatic, haloheteroaliphatic, aromatic, or an organic functional group. Dithiocarboxylic groups can be substituted with one or more groups other than hydrogen, such as aliphatic, heteroaliphatic, haloaliphatic, haloheteroaliphatic, aromatic, or an organic functional group.

Ester: —C(O)OR^{a} or —OC(O)R^{a}, wherein R^{a} is selected from aliphatic, heteroaliphatic, haloaliphatic, haloheteroaliphatic, aromatic, or an organic functional group. Ester groups can be substituted with one or more groups other than hydrogen, such as aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic. Ether groups can be substituted with one or more groups other than hydrogen, such as aliphatic, heteroaliphatic, haloaliphatic, haloheteroaliphatic, aromatic, or an organic functional group.

Fluorophore: A functional group or portion of a molecule that is capable of fluorescence. In some embodiments, a fluorophore can cause the molecule to fluoresce when exposed to an excitation source. Representative fluorophores can include, but are not limited to, a xanthene derivative (e.g., fluorescein, rhodamine, eosin, Texas red, Oregon green, or the like), cyanine or a cyanine derivative (e.g., indocarbocyanine, oxacarbocyanine, thiacarbocyanine, merocyanine, Cy3, or Cy5), a naphthalene derivative (e.g., dansyl, prodan, and the like), coumarin and derivatives thereof (e.g., hydroxycoumarin, aminocoumarin, methoxycoumarin, and the like), oxadiazole derivatives (e.g., pyridyloxazole, nitrobenzoxadiazole, benzoxadiazole, and the like), anthracene derivatives, pyrene derivatives (e.g., cascade blue), oxazine derivatives (e.g., Nile red, Nile blue, cresyl violet, and the like), acridine derivatives (e.g., auramine, crystal violet, malachite green, and the like), fluorone dyes (e.g., rhodamine, rhodol, methylrhodol), isoquinoline dyes (e.g., 2-(2-methoxyethyl)-1H-benzo[de]isoquinoline-1,3(2H)-dione), a naphthalimide compound (e.g., naphthalimide or 4-(2-methoxyethoxy)-N-butyl-1,8-naphthalimide), a chromenone dye (e.g., 4-methyl-2H-chromen-2-one), and tetrapyrrole derivatives (e.g., porphin, phthalocyanine, and the like) and in some embodiments can be methylrhodol, 2-(2-methoxyethyl)-1H-benzo[de]isoquinoline-1,3(2H)-dione, 4-methyl-2H-chromen-2-one, coumarin, naphthalimide, fluorescein, rhodamine, rhodol, Cy3, or Cy5. In some instances, compound embodiments of the present disclosure comprise a precursor to such fluorophore groups. Also, fluorophore compound embodiments can be described as heteroaryl and/or heteroaliphatic (e.g., heterocyclic) groups in the present disclosure.

Halo (or halide or halogen): Fluoro, chloro, bromo, or iodo. In some embodiments, halo can also include astatine.

Haloaliphatic: An aliphatic group wherein one or more hydrogen atoms, such as one to 10 hydrogen atoms, independently is replaced with a halogen atom, such as fluoro, bromo, chloro, or iodo. Haloaliphatic groups can be substituted with one or more groups other than hydrogen, such as aliphatic, heteroaliphatic, haloaliphatic, haloheteroaliphatic, aromatic, or an organic functional group.

Haloheteroaliphatic: A heteroaliphatic group wherein one or more hydrogen atoms, such as one to 10 hydrogen atoms, independently is replaced with a halogen atom, such as fluoro, bromo, chloro, or iodo. Haloheteroaliphatic groups can be substituted with one or more groups other than hydrogen, such as aliphatic, heteroaliphatic, haloaliphatic, haloheteroaliphatic, aromatic, or an organic functional group.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. Heteroaliphatic groups can be substituted with one or more groups other than hydrogen, such as aliphatic, heteroaliphatic, haloaliphatic, haloheteroaliphatic, aromatic, or an organic functional group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Hydroxyl: —OH.

Ketone: —C(O)R^{a}, wherein R^{a} is selected from aliphatic, heteroaliphatic, haloaliphatic, haloheteroaliphatic, aromatic, or an organic functional group. Ketone groups can be substituted with one or more groups other than hydrogen, such as aliphatic, heteroaliphatic, haloaliphatic, haloheteroaliphatic, aromatic, or an organic functional group.

Lower Alkyl: A saturated monovalent hydrocarbon having at least one carbon atom to 10 carbon atoms (C₁₋₁₀), such as 1, 2, 3, 4, 5, 6, 7, 8, 9, or 10 carbon atoms (C₁₋₂₅), wherein the saturated monovalent hydrocarbon can be derived from removing one hydrogen atom from one carbon atom of a parent compound (e.g., alkane). A lower alkyl group can be branched, straight-chain, or cyclic (e.g., cycloalkyl). Lower alkyl groups may be substituted with one or more groups other than hydrogen, such as aliphatic, heteroaliphatic, haloaliphatic, haloheteroaliphatic, aromatic, or an organic functional group.

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic acid or ester; phosphonate; phosphate; silyl ether; sulfonamide; sulfinyl; thial; or combinations thereof. Organic functional groups can be substituted with one or more groups other than hydrogen, such as aliphatic, heteroaliphatic, haloaliphatic, haloheteroaliphatic, aromatic, or an organic functional group.

Oxime: —CR^{a}═NOH, wherein R^{a} is hydrogen, aliphatic, heteroaliphatic, haloaliphatic, haloheteroaliphatic, aromatic, or an organic functional group. Oxime groups can be substituted with one or more groups other than hydrogen, such as aliphatic, heteroaliphatic, haloaliphatic, haloheteroaliphatic, aromatic, or an organic functional group.

Peroxy: —O—OR^{a} wherein R^{a} is hydrogen, aliphatic, heteroaliphatic, haloaliphatic, haloheteroaliphatic, aromatic, or an organic functional group. Peroxy groups can be substituted with one or more groups other than hydrogen, such as aliphatic, heteroaliphatic, haloaliphatic, haloheteroaliphatic, aromatic, or an organic functional group.

Phosphate: —O—P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{a} groups of the phosphate can be substituted with one or more groups other than hydrogen, such as aliphatic, heteroaliphatic, haloaliphatic, haloheteroaliphatic, aromatic, or an organic functional group.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{a} groups of the phosphonate group can be substituted with one or more groups other than hydrogen, such as aliphatic, heteroaliphatic, haloaliphatic, haloheteroaliphatic, aromatic, or an organic functional group.

Phosphorophore: A compound or complex capable of phosphorescence.

Photocatalyst: Refers to a molecule that is capable of absorbing light and transferring energy to a different molecule, in order to trigger a chemical reaction. Exemplary photocatalysts include porphyrin species (with or without metals bound), as well as methylene blue, rose Bengal, and various complexes of iridium, ruthenium, and rhodium.

Polymer: An organic or mixed organic/inorganic molecule comprising repeating monomer subunits. In some embodiments, a polymer can comprise repeating monomer subunits that are the same (e.g., a homopolymer) or different (e.g., a copolymer). In some embodiments a polymer can comprise a crosslinked polymer.

Quantum dot: A nanoscale particle that exhibits size-dependent electronic and optical properties due to quantum confinement. Quantum dots have, for example, been constructed of semiconductor materials (e.g., cadmium selenide and lead sulfide) and from crystallites (grown via molecular beam epitaxy), etc. Quantum dots also can include alloy quantum dots, such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN quantum dots.

Silyl Ether: —OSiR^{b}R^{c}, wherein each of R^{b} and R^{c} independently is selected from hydrogen, aliphatic, heteroaliphatic, haloaliphatic, haloheteroaliphatic, aromatic, or an organic functional group. Silyl ether groups can be substituted with one or more groups other than hydrogen, such as aliphatic, heteroaliphatic, haloaliphatic, haloheteroaliphatic, aromatic, or an organic functional group.

Sulfinyl: —S(O)R^{a}, wherein R^{a} is selected from hydrogen, aliphatic, heteroaliphatic, haloaliphatic, haloheteroaliphatic, aromatic, or an organic functional group. Sulfinyl groups can be substituted with one or more groups other than hydrogen, such as aliphatic, heteroaliphatic, haloaliphatic, haloheteroaliphatic, aromatic, or an organic functional group.

Sulfonyl: —SO₂R^{a}, wherein R^{a} is selected from hydrogen, aliphatic, heteroaliphatic, haloaliphatic, haloheteroaliphatic, aromatic, or an organic functional group. Sulfonyl groups can be substituted with one or more groups other than hydrogen, such as aliphatic, heteroaliphatic, haloaliphatic, haloheteroaliphatic, aromatic, or an organic functional group.

Sulfonamide: —SO₂NR^{b}R^{c} or —N(R^{b})SO₂R^{c}, wherein each of R^{b} and R^{c} independently is selected from hydrogen, aliphatic, heteroaliphatic, haloaliphatic, haloheteroaliphatic, aromatic, or an organic functional group. Sulfonamide groups can be substituted with one or more groups other than hydrogen, such as aliphatic, heteroaliphatic, haloaliphatic, haloheteroaliphatic, aromatic, or an organic functional group.

Sulfonate: —SO₃⁻, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Thial: —C(S)H.

Thiocarboxylic acid: —C(O)SH, or —C(S)OH.

Thiocyanate: —S—CN or —N═C═S.

Thioester: —C(O)SR^{a} wherein R^{a} is selected from hydrogen, aliphatic, heteroaliphatic, haloaliphatic, haloheteroaliphatic, aromatic, or an organic functional group. Thioester groups can be substituted with one or more groups other than hydrogen, such as aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S-heteroaryl; or -aliphatic-S-aliphatic, -aliphatic-S-aromatic, -aromatic-S-aliphatic, or -aromatic-S-aromatic. Thioether groups can be substituted with one or more groups other than hydrogen, such as aliphatic, heteroaliphatic, haloaliphatic, haloheteroaliphatic, aromatic, or an organic functional group.

Thioketone: —C(S)R^{a} wherein R^{a} is selected from hydrogen, aliphatic, heteroaliphatic, haloaliphatic, haloheteroaliphatic, aromatic, or an organic functional group. Thioketone groups can be substituted with one or more groups other than hydrogen, such as aliphatic, heteroaliphatic, haloaliphatic, haloheteroaliphatic, aromatic, or an organic functional group.

Thionoester: —C(S)OR^{a} wherein R^{a} is selected from hydrogen, aliphatic, heteroaliphatic, haloaliphatic, haloheteroaliphatic, aromatic, or an organic functional group. Thionoester groups can be substituted with one or more groups other than hydrogen, such as aliphatic, heteroaliphatic, haloaliphatic, haloheteroaliphatic, aromatic, or an organic functional group.

### II. Introduction

Crosslinked PDCPD is used in a wide variety of commercial applications; however, it is not chemically tunable and has other drawbacks that limit its use in particular industries. There are some methods known in the art that permit adding chemical functionality to the surface of PDCPD following polymerization and crosslinking; however, because PDCPD cannot be melted or dissolved due to the high crosslink density, these methods are limited to manipulating the polymer surface and cannot be used to tune the polymer's bulk mechanical properties.

An alternative, but less extensively explored strategy, is to chemically modify the dicyclopentadiene monomer in such a way that it can still participate in both the primary polymerization reaction and subsequent crosslinking events, but will incorporate a desirable functional group into the final material. This approach is challenging because the dicyclopentadiene monomer is itself a dimer of two cyclopentadiene sub-monomers, joined together through a [4+2] cycloaddition. Dicyclopentadiene incorporates both a strained norbornene-type alkene, which is required for primary polymerization, and an unstrained cyclopentene-type alkene, which participates in subsequent crosslinking events (along with, potentially, the backbone alkenes from the polymerization process). Adding functionality in the vicinity of the strained alkene will negatively affect the primary polymerization. Adding functionality near (or on) the unstrained alkene may be better tolerated (although it may change the mechanism of polymer crosslinking), but selectively manipulating the unstrained (and therefore less reactive) alkene in preference to the more strained (and therefore more reactive) alkene is challenging to accomplish chemically. As such, strategies currently known in the art for making chemically functionalized dicyclopentadiene monomers are limited, and products resulting therefrom still have associated drawbacks (e.g., thermal instability due to the presence of a labile allylic functional group and/or need to the need to separate regioisomeric products of similar molecular weight and polarity). Additionally, some such methods rely on using expensive reagents (e.g., SeO₂), often at stochiometric amounts, which leads to undesirably high costs in preparing monomers for polymerization.

Disclosed herein are embodiments of a monomer (along with polymer embodiments formed therefrom) that exhibit desirable properties and that can be made using an efficient method with low-cost reagents. Monomer embodiments of the present disclosure have a unique skeleton that provides the ability for modifications to the skeleton pre- or post-polymerization and/or pre- or post-crosslinking. Moreover, the unique skeleton of the monomer provides the ability to tune the polymer's bulk mechanical properties and the method furthers this gain by providing the ability to mix and match different monomers that become part of the skeleton during polymerization (or making structural changes to different monomer subunits of the polymer after polymerization and/or crosslinking). In addition, monomer embodiments disclosed herein can be made with high regioselectivity, and the polymer embodiments obtained therefrom exhibit high thermal stability that cannot be achieved by polymers made from the monomer embodiments presently known in the art. Other benefits and advantages of the present disclosure are discussed herein.

### III. Monomer and Polymer Embodiments

Disclosed herein are embodiments of a monomer that can be used in an olefin metathesis reaction to form a polymer. In particular embodiments, the monomer has a structure according to Formula I.

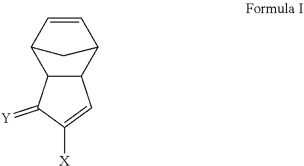

With reference to Formula I, X is selected from hydrogen, deuterium, halogen (e.g. Cl, Br, F, or I), aliphatic, haloaliphatic, haloheteroaliphatic, heteroaliphatic, aromatic, an organic functional group, a detectable moiety (e.g., a chromogen, a fluorophore, a phosphorophore, a quantum dot, or the like), an active component, or any combination of such groups; and Y is selected from O; S; Se; or NR^{c}, wherein R^{c} is hydrogen, —OR^{d}, or —NR^{d}R^{e}, wherein, for —OR^{d} and —NR^{d}R^{e}, R^{d} and R^{e} independently are selected from hydrogen, aliphatic, haloaliphatic, haloheteroaliphatic, heteroaliphatic, aromatic, or an organic functional group. In some embodiments, if Y is O, then X is other than hydrogen. In particular embodiments, X is hydrogen, deuterium, Cl, Br, I, alkyl, alkynyl, alkenyl, heteroalkyl, heteroalkenyl, heteroalkynyl, aryl, heteroaryl, hydroxyl, aldehyde, ester, amide, ketone, a fluorophore, an active component, or a combination of any such groups; and Y is O; S; N-aromatic; or N—OR^{d} or N—NR^{d}R^{e}, wherein, for-OR^{d} and —NR^{d}R^{e}, R^{d} and R^{e} independently are selected from hydrogen, alkyl, heteroalkyl, aryl, or heteroaryl. In yet additional embodiments, X is hydrogen, deuterium, Br, I, lower alkyl, —C≡CR^{d} (wherein R^{d} is selected from hydrogen, aliphatic, haloaliphatic, haloheteroaliphatic, heteroaliphatic, aromatic, or an organic functional group), —C═CR^{d}R^{e} (wherein R^{d} and R^{e} independently are selected from hydrogen, aliphatic, haloaliphatic, haloheteroaliphatic, heteroaliphatic, aromatic, or an organic functional group), thioether, ether, amine, hydroxyl, aldehyde, ester (lower alkyl ester, such as methyl ester, ethyl ester, propyl ester, and the like), amide, ketone, an aryl ring system comprising from 6 ring atoms to 10 ring atoms (such as 6 ring atoms to 8 ring atoms, including, but not limited to, phenyl, naphthyl, or the like), a heteroaryl ring system comprising from 5 ring atoms to 10 ring atoms, with at least one heteroatom (such as 5 ring atoms to 8 ring atoms, or 5 ring atoms to 7 ring atoms, including, but not limited to, furanyl, thiophenyl, pyrrolyl, pyridinyl, or pyrimidinyl), fluorescein, rhodamine, BODIPY, an antibacterial agent, an antifungal agent, an anticancer agent, a peptide promoting cellular adhesion, a signaling factor controlling cellular growth or motility, a vitamin, a cofactor, or any combination of any such groups. In yet additional embodiments, Y is O, S, Se, N-phenyl, N-pyridinyl, N—OH, N—OMe, N—OEt, N—NH₂, N—NHMe, N—N(Me)₂, N—N(Et)₂, or N—N(Me)Et. In an independent embodiment, the monomer is not endo-dicyclopentadienone, wherein, if Y is O, then X is hydrogen (also known as (3aS,4R,7S,7aR)-3a,4,7,7a-tetrahydro-1H-4,7-methanoinden-1-one) or is not exo-dicyclopentadienone wherein, if Y is O, then X is hydrogen (also known as (3aR,4R,7S,7aS)-3a,4,7,7a-tetrahydro-1H-4,7-methanoinden-1-one). However, in particular embodiments, such a monomer may be used to make a polymer according to the present disclosure.

In some embodiments, the monomer of Formula I is an endo diastereomer or an exo diastereomer or a mixture of endo and exo diastereomers. Exemplary, non-limiting, species of the monomer are provided below.

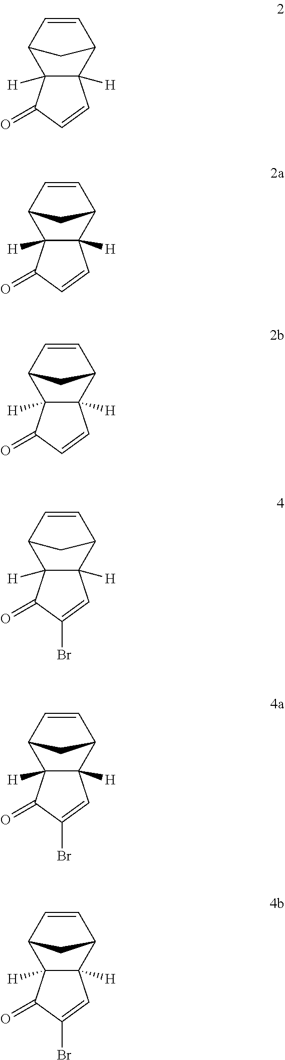 [SMILES: O=C1C=CC2C3C=CC(C3)C12 ; O=C1C=C[C@@H]2[C@H]1[C@@H]1C=C[C@H]2C1 ; O=C1C=C[C@H]2[C@@H]1[C@@H]1C=C[C@H]2C1 ; O=C1C(Br)=CC2C3C=CC(C3)C12 ; O=C1C(Br)=C[C@@H]2[C@H]1[C@@H]1C=C[C@H]2C1 ; O=C1C(Br)=C[C@H]2[C@@H]1[C@@H]1C=C[C@H]2C1]

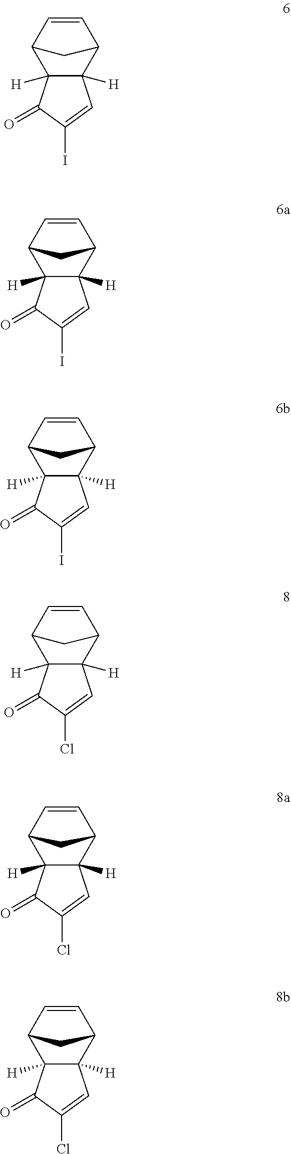 [SMILES: O=C1C(I)=CC2C3C=CC(C3)C12 ; O=C1C(I)=C[C@@H]2[C@H]1[C@@H]1C=C[C@H]2C1 ; O=C1C(I)=C[C@H]2[C@@H]1[C@@H]1C=C[C@H]2C1 ; O=C1C(Cl)=CC2C3C=CC(C3)C12 ; O=C1C(Cl)=C[C@@H]2[C@H]1[C@@H]1C=C[C@H]2C1 ; O=C1C(Cl)=C[C@H]2[C@@H]1[C@@H]1C=C[C@H]2C1]

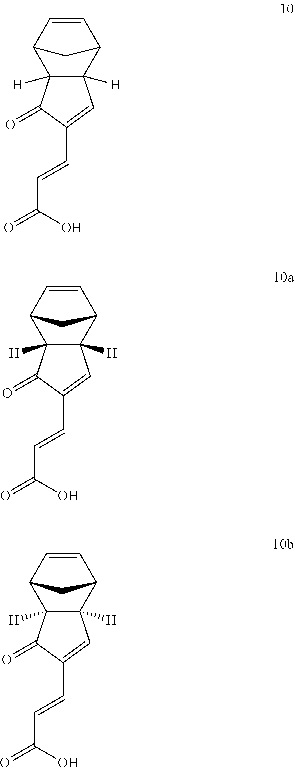 [SMILES: O=C(O)/C=C/C1=CC2C3C=CC(C3)C2C1=O ; O=C(O)/C=C/C1=C[C@@H]2[C@H](C1=O)[C@@H]1C=C[C@H]2C1 ; O=C(O)/C=C/C1=C[C@H]2[C@@H](C1=O)[C@@H]1C=C[C@H]2C1]

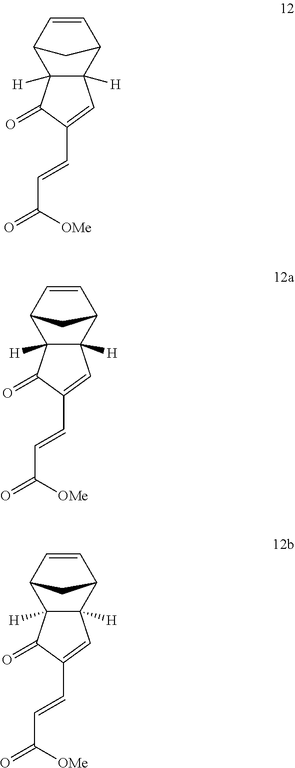 [SMILES: COC(=O)/C=C/C1=CC2C3C=CC(C3)C2C1=O ; COC(=O)/C=C/C1=C[C@@H]2[C@H](C1=O)[C@@H]1C=C[C@H]2C1 ; COC(=O)/C=C/C1=C[C@H]2[C@@H](C1=O)[C@@H]1C=C[C@H]2C1]

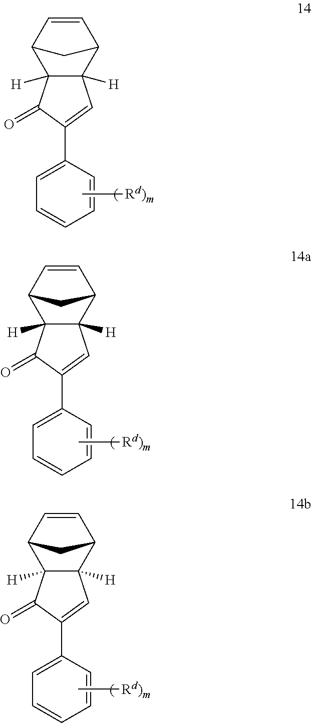

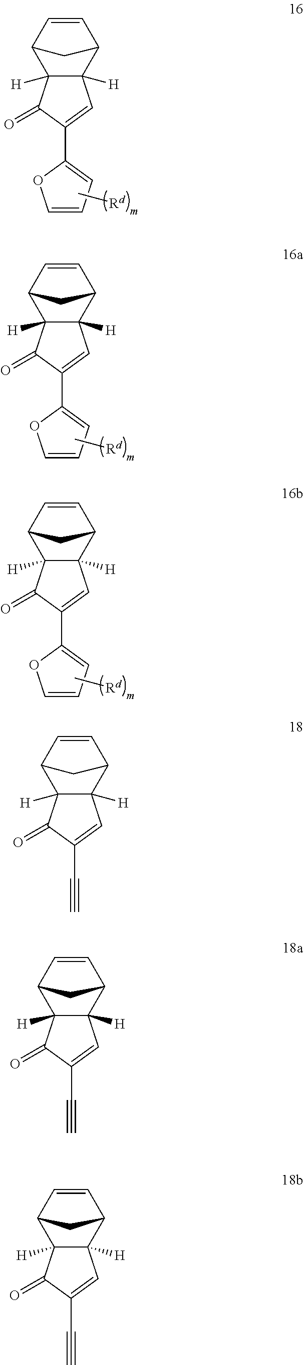

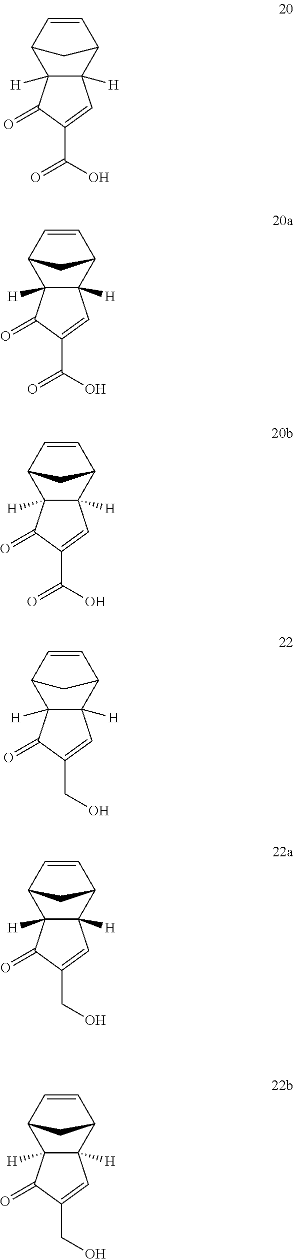 [SMILES: O=C(O)C1=CC2C3C=CC(C3)C2C1=O ; O=C(O)C1=C[C@@H]2[C@H](C1=O)[C@@H]1C=C[C@H]2C1 ; O=C(O)C1=C[C@H]2[C@@H](C1=O)[C@@H]1C=C[C@H]2C1 ; O=C1C(CO)=CC2C3C=CC(C3)C12 ; O=C1C(CO)=C[C@@H]2[C@H]1[C@@H]1C=C[C@H]2C1 ; O=C1C(CO)=C[C@H]2[C@@H]1[C@@H]1C=C[C@H]2C1]

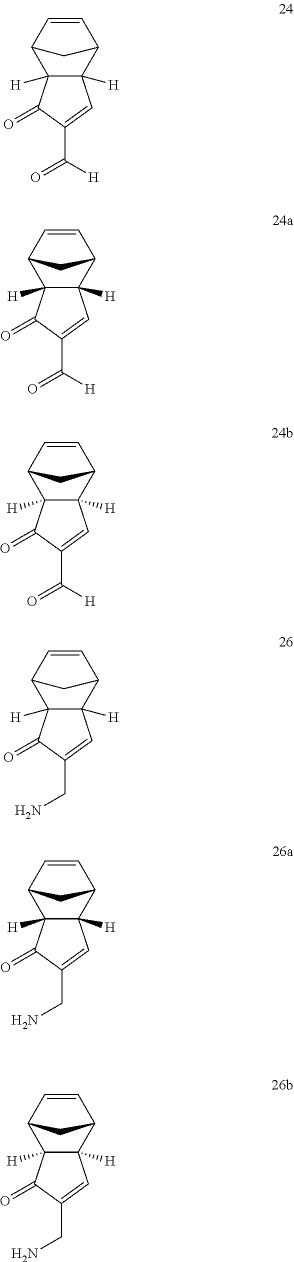 [SMILES: O=CC1=CC2C3C=CC(C3)C2C1=O ; O=CC1=C[C@@H]2[C@H](C1=O)[C@@H]1C=C[C@H]2C1 ; O=CC1=C[C@H]2[C@@H](C1=O)[C@@H]1C=C[C@H]2C1 ; NCC1=CC2C3C=CC(C3)C2C1=O ; NCC1=C[C@@H]2[C@H](C1=O)[C@@H]1C=C[C@H]2C1 ; NCC1=C[C@H]2[C@@H](C1=O)[C@@H]1C=C[C@H]2C1]

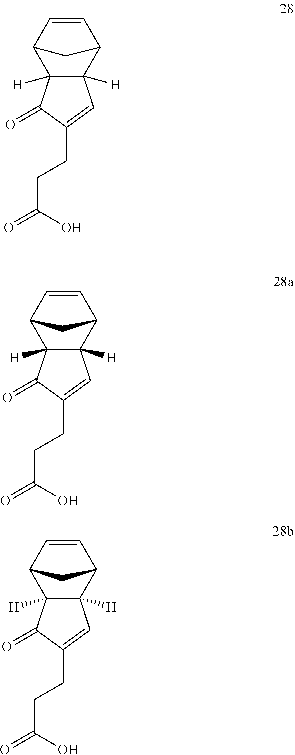

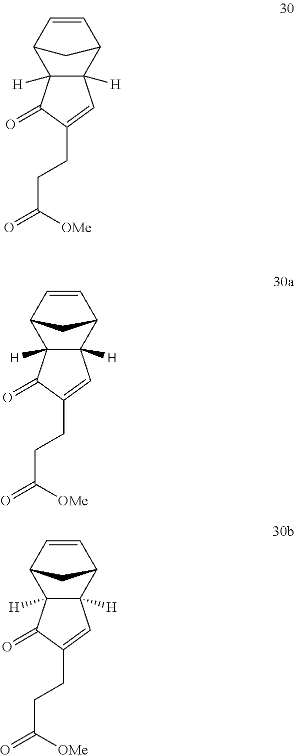

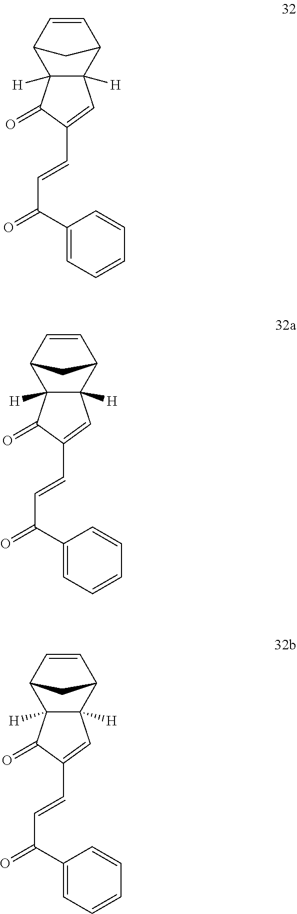 [SMILES: O=C(/C=C/C1=CC2C3C=CC(C3)C2C1=O)c1ccccc1 ; O=C(/C=C/C1=C[C@@H]2[C@H](C1=O)[C@@H]1C=C[C@H]2C1)c1ccccc1 ; O=C(/C=C/C1=C[C@H]2[C@@H](C1=O)[C@@H]1C=C[C@H]2C1)c1ccccc1]

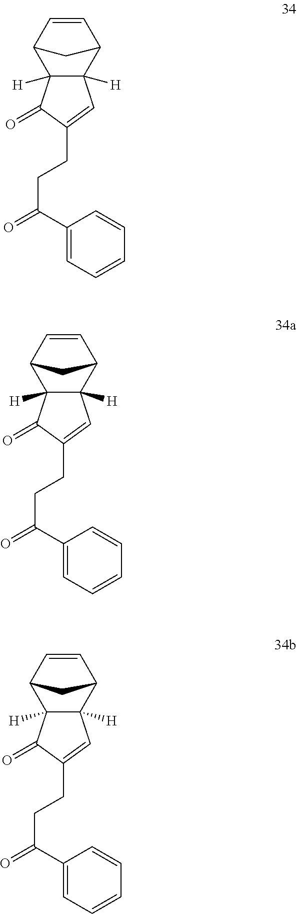 [SMILES: O=C(CCC1=CC2C3C=CC(C3)C2C1=O)c1ccccc1 ; O=C(CCC1=C[C@@H]2[C@H](C1=O)[C@@H]1C=C[C@H]2C1)c1ccccc1 ; O=C(CCC1=C[C@H]2[C@@H](C1=O)[C@@H]1C=C[C@H]2C1)c1ccccc1]

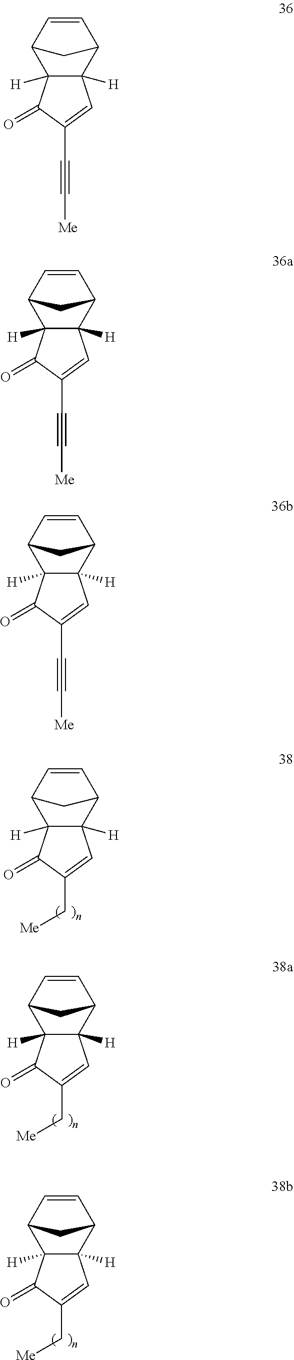

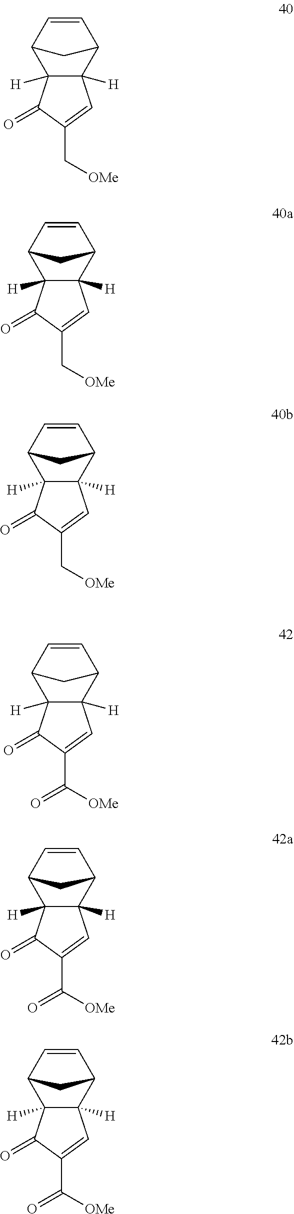

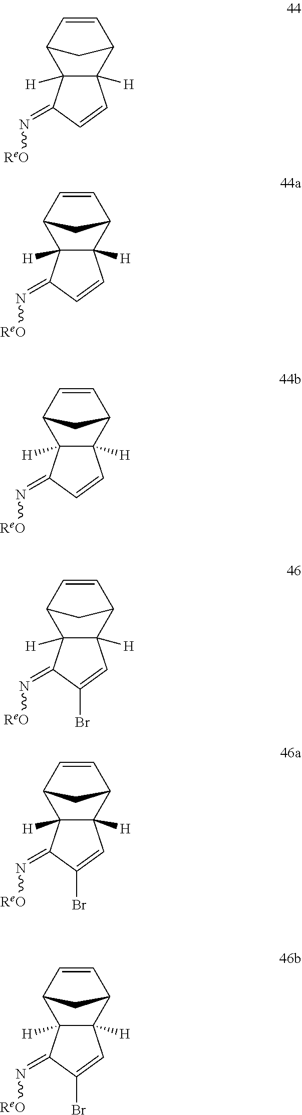

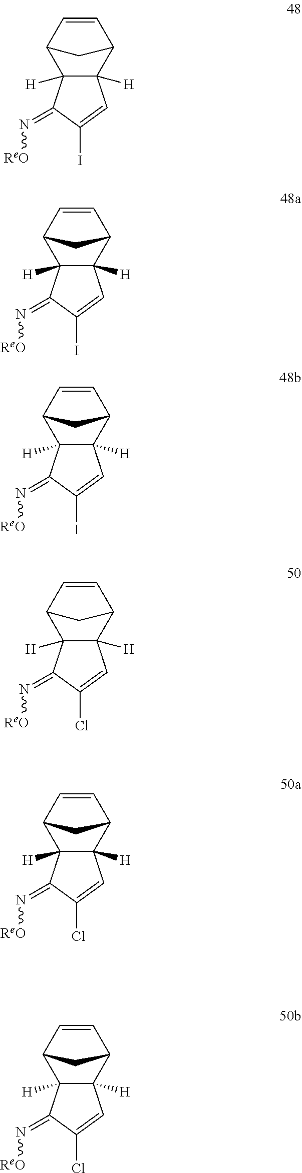

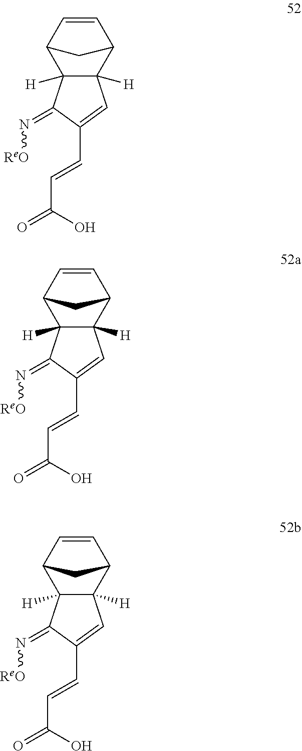

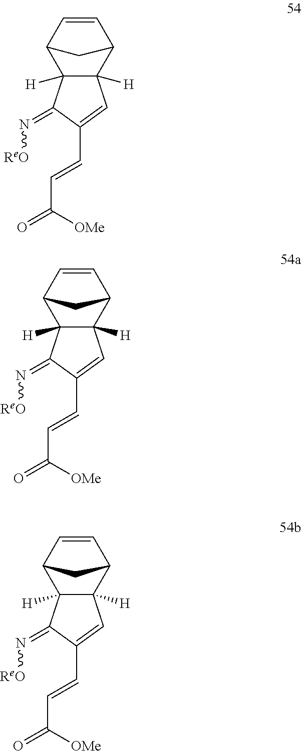

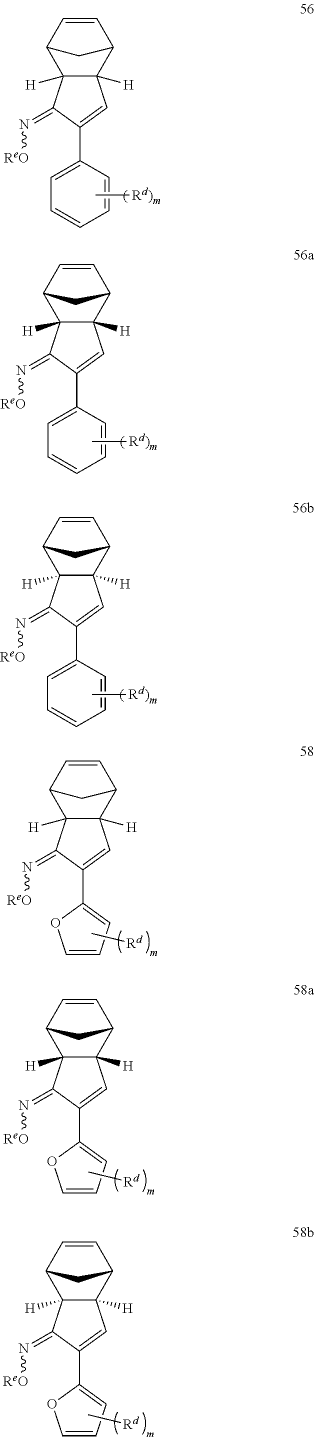

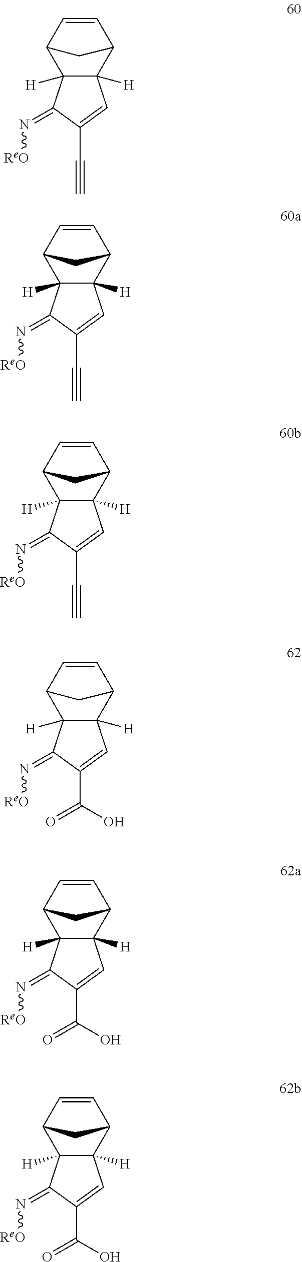

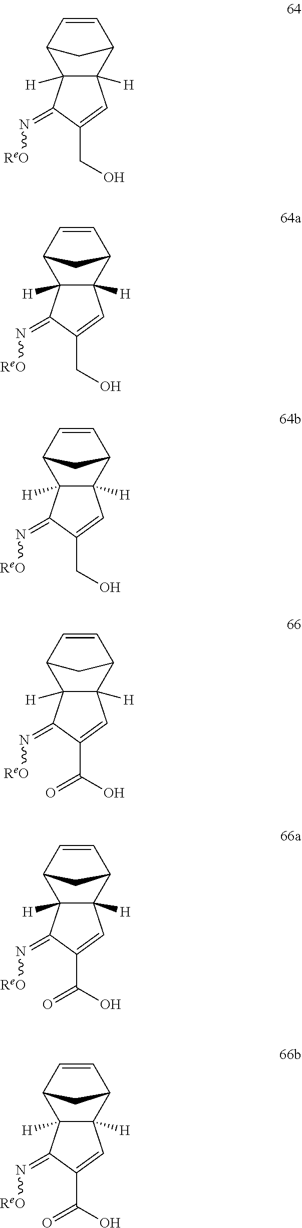

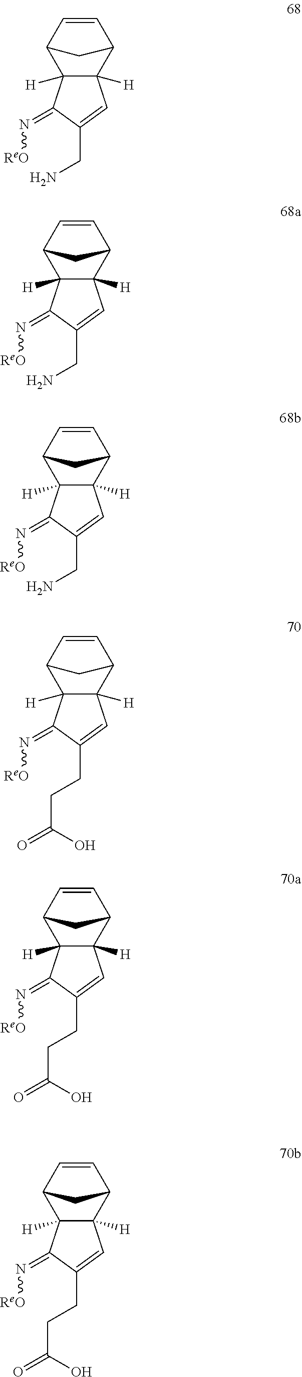

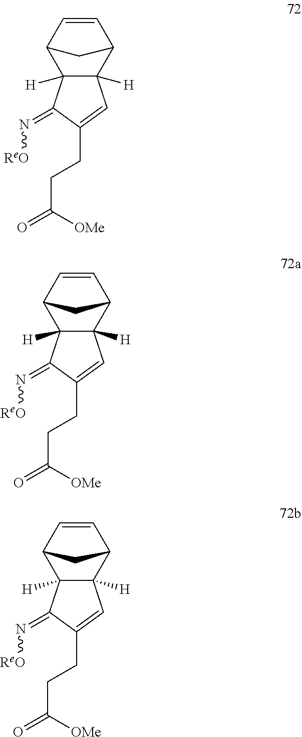

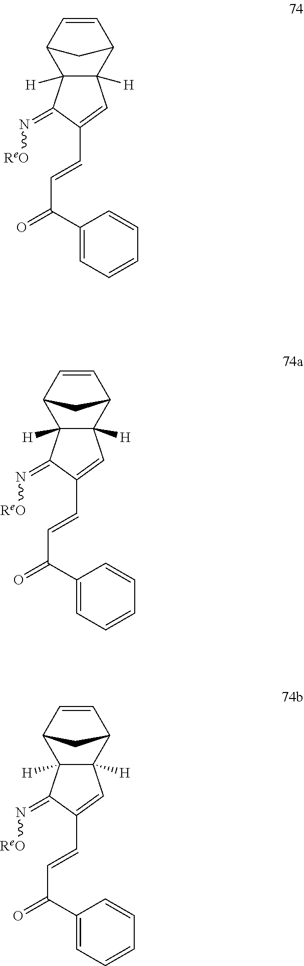

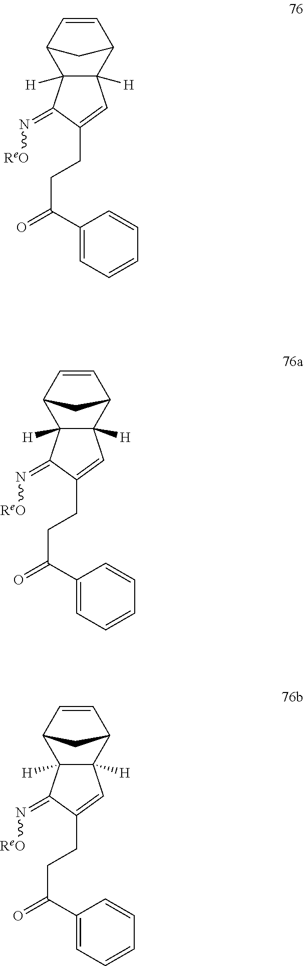

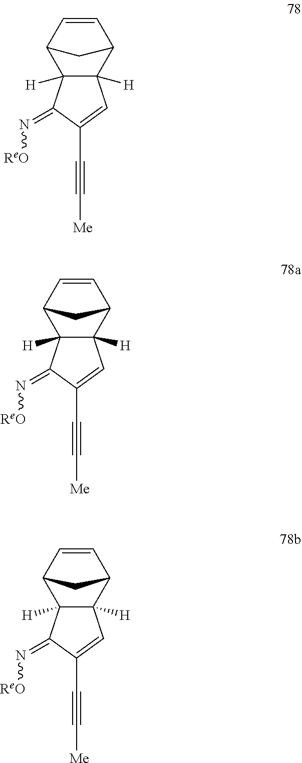

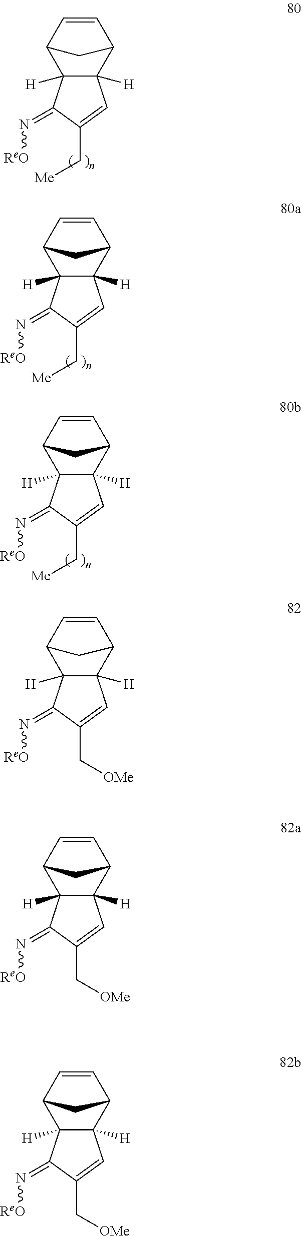

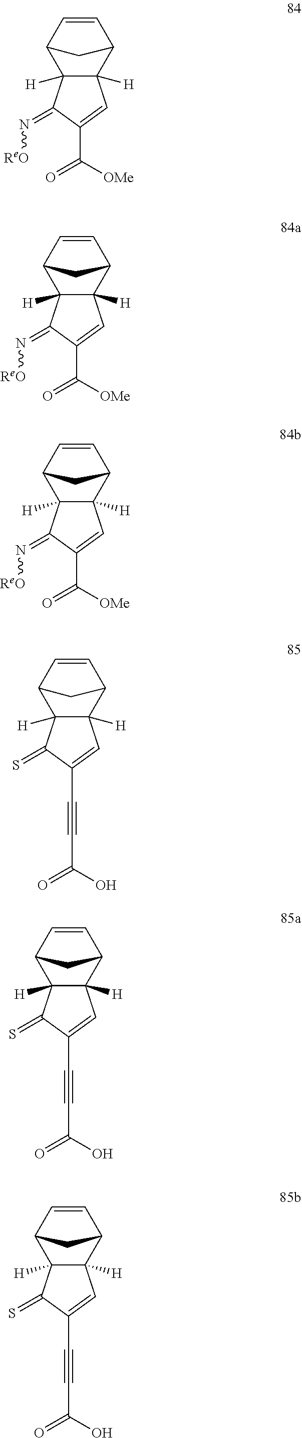

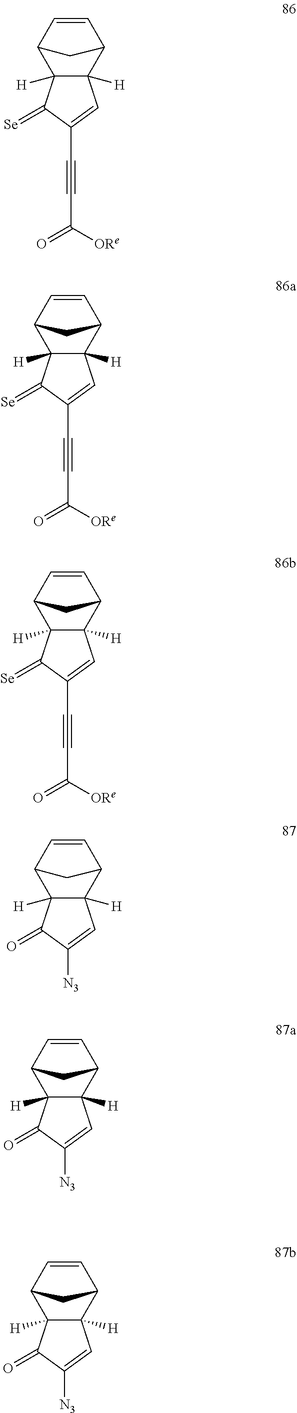

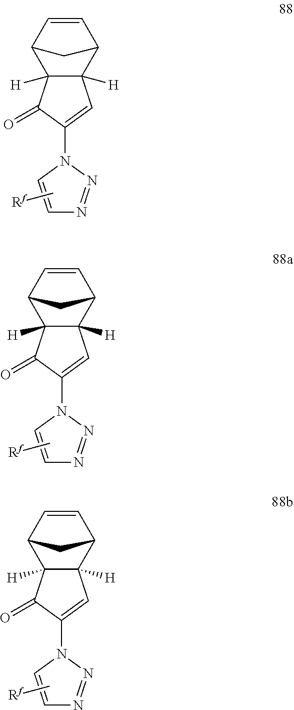

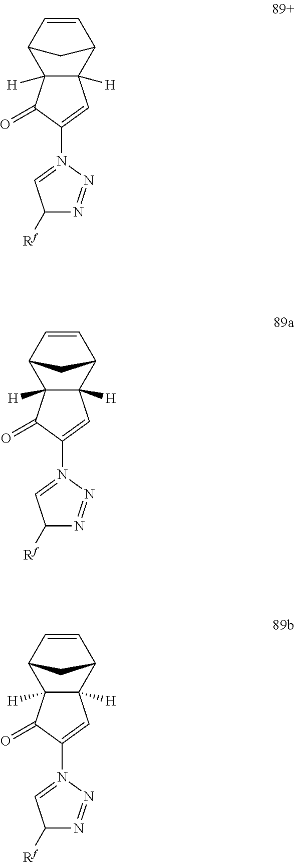

With reference to the above-illustrated monomer embodiments, the following substituent recitations can apply for the variable groups included in the formulas:
each R^{d} and R^{e} independently can be selected from hydrogen, aliphatic, haloaliphatic, haloheteroaliphatic, heteroaliphatic, aromatic, or an organic functional group and in particular embodiments can be any of the particular groups recited above for Formula I and/or any particular groups included in definitions provided herein for aliphatic, haloaliphatic, haloheteroaliphatic, heteroaliphatic, aromatic, and/or organic functional groups;
R^{f} can be an active component or a detectable moiety;
each n can be an integer selected from 0 to 25, such as 0 to 20, or 0 to 10, or 0 to 6, or 1, 2, 3, 4, 5, 6, 7, 8, 9, or 10; and
each m can be an integer selected from 0 to 5, such as 0, 1, 2, 3, 4, or 5.

Also disclosed herein are embodiments of polymers that can be made using a monomer according to Formula I (or any other embodiments described above). In some embodiments, the polymer can be a linear homopolymer, wherein the polymer is formed from the same monomer having a structure according to Formula I. In some other embodiments, the polymer can be a linear copolymer, wherein the polymer is formed from two or more monomer subunits that are structurally distinct, with at least one of the monomer subunits being a monomer according to Formula I. In some embodiments, the copolymer comprises at least one monomer according to Formula I and a different monomer subunit which can be selected from (i) cyclic olefin-containing compounds, such as cyclobutene, cyclopentene, cycloheptene, cyclooctene, cyclononene, cyclodecene, cyclododecene, norbornene, cyclooctadiene, cyclononadiene, norbornadiene, and the like, as well as functionalized forms of such cyclic olefin-containing compounds; (ii) acyclic dienes; or (iii) unfunctionalized dicyclopentadiene. In particular embodiments, the polymer has a structure comprising at least one monomer subunit according to Formula IA, wherein each X and each Y independently can be selected from X and Y groups, respectively, as provided herein for Formula I. In some embodiments, each Y independently can be modified to comprise a detectable moiety (e.g., a chromogen, a fluorophore, a phosphorophore, a quantum dot, or the like) or an active component after the polymer is formed (and/or before or after any crosslinking, as discussed herein).

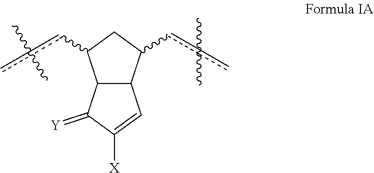

In some embodiments, the polymer can comprise a polymer end-capping group, such as is illustrated in Formula IB. As illustrated in Formula IIB, the polymer end-capping group typically is attached to C^{a} or C^{b} of Formula IIB (each of which further bears a hydrogen atom to provide a complete valency for the sp² hybridized C^{a} or C^{b} group). In some embodiments, an end-capping group can be obtained from the catalyst or initiator employed during polymerization and/or by quenching polymerization with an additional chemical reagent. In some embodiments, the end-capping group can be selected from hydrogen, aliphatic, aromatic, haloaliphatic, haloheteroaliphatic, heteroaliphatic, or an organic functional group. In particular embodiments, the end-capping group is hydrogen, alkyl, or phenyl. In some embodiments, other suitable end-capping groups can be used and would be recognized by a person having at least ordinary skill in the art with the benefit of the present disclosure.

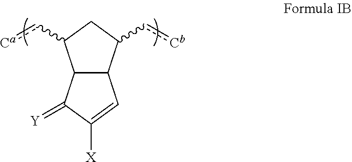

In particular embodiments, the polymer can be a homopolymer having a structure according to Formula II.

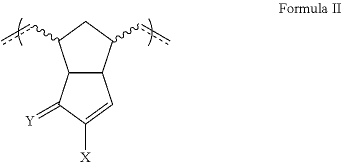

With reference to Formula II, each of X and Y independently for each occurrence can be as recited above for Formula I and/or Y can further comprise a detectable moiety (e.g., a chromogen, a fluorophore, a phosphorophore, a quantum dot, or the like) or an active component. Any number of the monomer units can be included in the polymer, such as 2 or more monomer units, or 4 or more monomer units, or 8 or more monomer units, or 10 or more monomer units. In exemplary embodiments, the polymer can comprise 2 to 1000 monomer units, such as 10 to 500 monomer units. In particular embodiments, the polymer can have a molecular weight ranging from 2,000 Daltons to 200,000 Daltons. In some embodiments, the polymer can be a polymer comprising an unsaturated backbone (e.g., see Formula IIA) or a polymer comprising a saturated backbone (e.g., see Formula IIB).

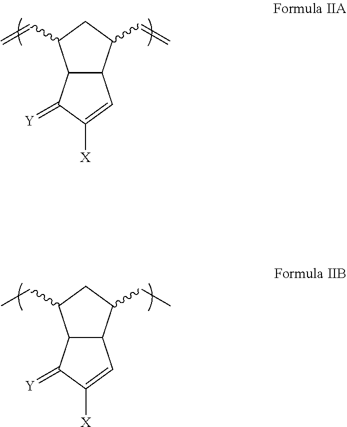

In yet additional embodiments, the polymer can be a copolymer having a structure according to Formula IIIA or Formula IIIB.

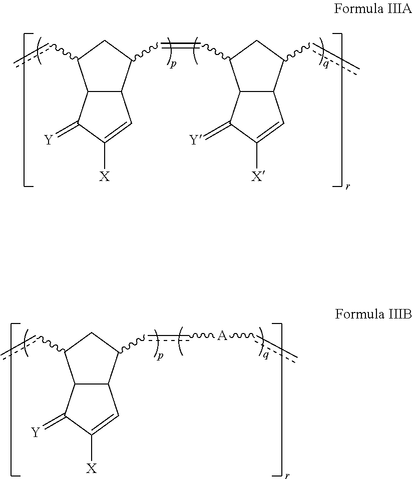

As with the polymer embodiments described above, the copolymer can be a copolymer having an unsaturated backbone (e.g., see Formula IIIA′ and Formula IIIB′) or a polymer comprising a saturated backbone (e.g., see Formula IIIA″ or Formula IIIB″).

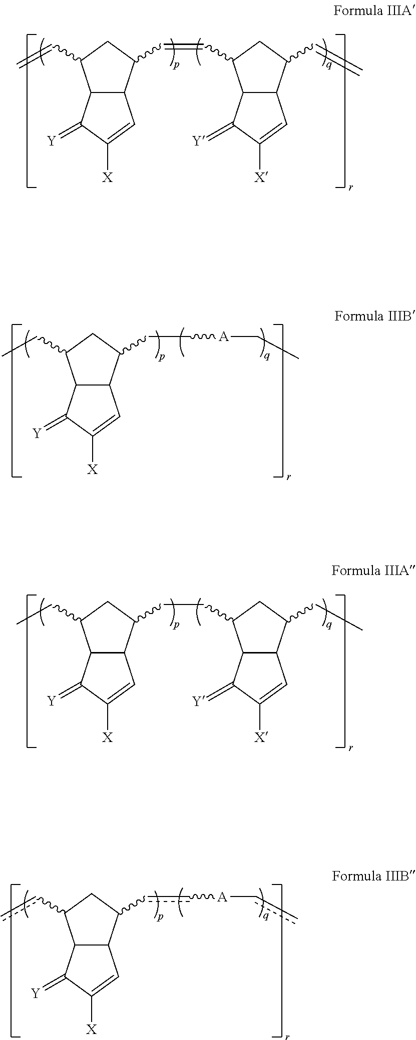

With reference to any of Formulas IIIA, IIIA′, and IIIA″, each of Y and X independently for each occurrence can be as recited for Formula I and each of Y′ and X independently for each occurrence' can be as recited for X and Y of Formula I, provided that either (i) at least one X and at least one X′ are different from one another; or (ii) at least one Y and at least one Y′ are different from one another; each p independently can be an integer of at least 1; each q independently can be an integer of at least 1; and r can be an integer of at least 1. In particular embodiments, each p independently can be 1 to 500 (e.g., 1 to 250); each q independently can be 1 to 500 (e.g., 1 to 250); and r can be an integer of at least 1 to 500. In some embodiments, each Y and each Y′ independently for each occurrence can comprise a detectable moiety (e.g., a chromogen, a fluorophore, a phosphorophore, a quantum dot, or the like) or an active component. With reference to any of Formulas IIIB, IIIB′, and IIIB″, each of Y and X independently for each occurrence can be as recited for Formula I, and/or each Y independently for each occurrence can comprise a detectable moiety (e.g., a chromogen, a fluorophore, a phosphorophore, a quantum dot, or the like) or an active component; component “A” can be the product after a metathesis reaction between a cyclic olefin-containing compound, an acyclic diene, or unfunctionalized dicyclopentadiene with a monomer subunit of Formula I (or a second cyclic olefin-containing compound, an acyclic diene, or unfunctionalized dicyclopentadiene); each p independently can be an integer of at least 1; each q independently can be an integer of at least 1; and r can be an integer of at least 1. In particular embodiments, “A” can be an aliphatic group, such as an acyclic aliphatic group (e.g., a linear alkyl change or branched alkyl chain) or a cyclic aliphatic group (e.g., a bicyclic group).

In yet additional embodiments, the polymer can be a crosslinked homopolymer comprising at least one molecular crosslink that links monomer subunits of the homopolymer (such as an intrapolymer crosslink formed between a monomer subunit of the same polymer) or that links monomer subunits of two or more separate homopolymers (such as an interpolymer crosslink between two different monomer subunits of two different polymers). In such embodiments, if two separate homopolymers are crosslinked, each homopolymer can be the same or different as the other. In yet additional embodiments, the polymer can be a crosslinked copolymer comprising at least one molecular crosslink that links monomer subunits of the copolymer (such as an intrapolymer crosslink formed between a monomer subunit of the same polymer) or that links monomer subunits of two or more separate copolymers (such as an interpolymer crosslink between two different monomer subunits of two different polymers). In such embodiments, if two separate copolymers are crosslinked, each copolymer can be the same or different as the other. In some embodiments, crosslinking can be accomplished by subjecting a polymer (e.g., a copolymer or a homopolymer) to a thermal or chemical crosslinking reaction using conditions for crosslinking (e.g., exposing the polymer to air, radical crosslinking conditions, anionic crosslinking conditions, and/or a crosslinking reagent), or it can take place during polymerization. A representative example of a crosslinked polymer structure is illustrated below (Formula IV); however, it should be recognized that the present disclosure is not limited to this specific structure and the illustrated monomer subunits can be replaced with (i) other structures obtained from cross-metathesis of a monomer of Formula I with a cyclic olefin-containing compound, an acyclic diene, or unfunctionalized dicyclopentadiene, such as with an A group of Formula IIIB above; or (ii) monomers wherein the illustrated X and Y groups are different from other monomers in the polymer (e.g., such as which can be the case in a crosslinked copolymer as described above). Further, while only one exemplary crosslink is illustrated in Formula IV, other crosslinks are contemplated, such as additional crosslinks that can be formed between other monomers in the polymer by way of carbon-carbon bond formation between vinylic carbon atoms of the monomers' ring systems. Also, with reference to Formula IV, each X and each Y independently, for each occurrence, can be as defined herein for any of Formulas I, II, or IIIA and IIIB.

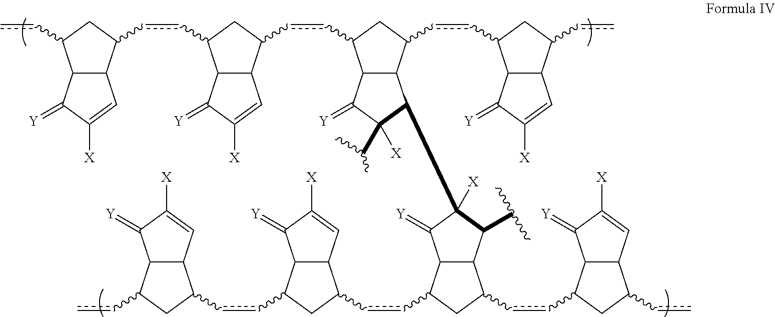

In some embodiments, the polymer can have a structure according to any of the formulas illustrated below, wherein the X and Y groups of the illustrated monomer subunit independently and for each occurrence can be selected from groups defined as X and Y groups for Formula I; or Y can, independently for each occurrence, comprise a detectable moiety (e.g., a chromogen, a fluorophore, a phosphorophore, a quantum dot, or the like) or an active component. Any “cargo” group illustrated below can be a detectable moiety or an active component; and any R or R′ groups are selected from aliphatic, haloaliphatic, haloheteroaliphatic, heteroaliphatic, aromatic, or an organic functional group. While the embodiments illustrated below are shown as formulas contemplating homopolymers and crosslinked homopolymers, the present disclosure also contemplates embodiments wherein the polymer is a copolymer (or crosslinked copolymer) comprising at least one monomer subunit having a structure as shown in the monomer subunits of the polymers below.

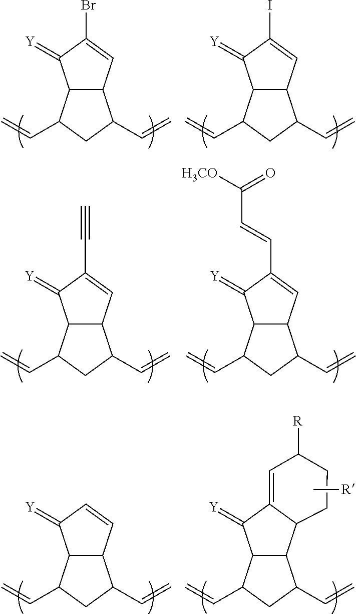

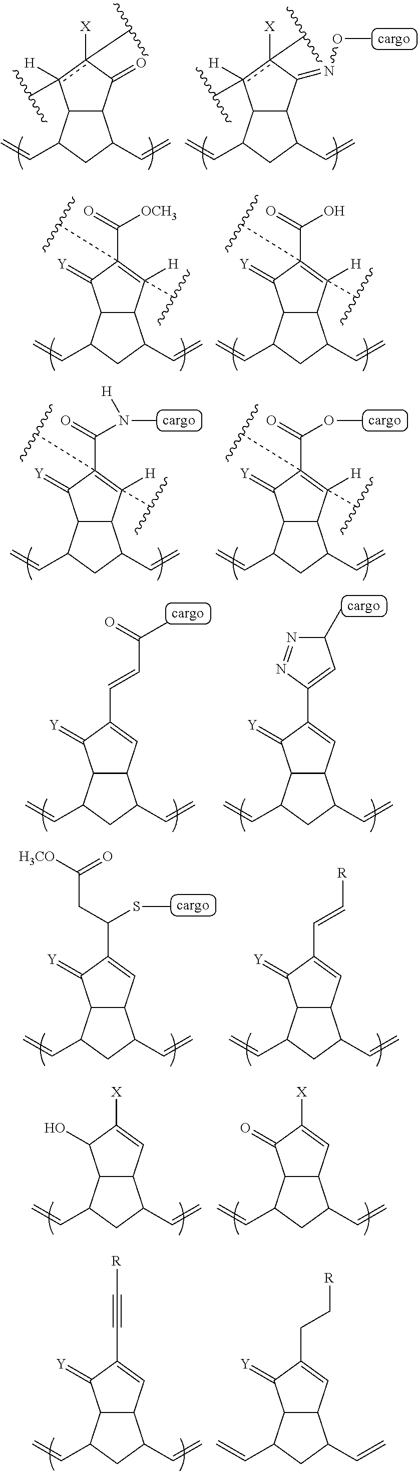

Exemplary, but non-limiting, polymer species are provided by the Examples section.

### IV. Method of Making Monomer and Polymer Embodiments

Method embodiments for making monomer and polymer compounds disclosed herein are described. In particular embodiments and as illustrated in Scheme 1, a monomer embodiment according to the present disclosure can be made by first oxidizing the endo or exo diastereomer of dicyclopentadiene (100) to the corresponding α,β-unsaturated C═O group of Formula I (102). This step is carried out photochemically using low loadings of commercially available photocatalysts and in turn renders the unstrained alkene of Formula I electrophilic. This facilitates further functionalization of the monomer with various types of X groups using methods described herein (104), such as nucleophilic catalysis to install a vinyl X group, as illustrated in Scheme 1. With reference to Scheme 1, the monomer can be the endo monomer or the exo monomer and, in particular embodiments, X can be halogen or —CH₂OH.

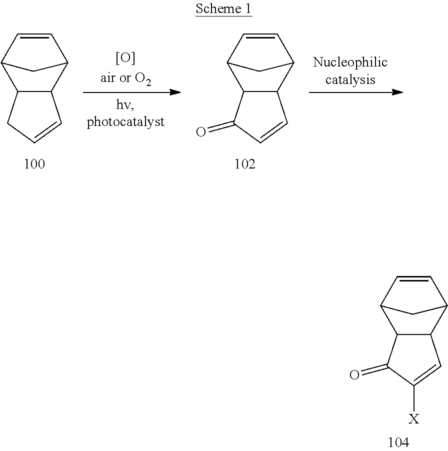

After the functionalized monomer 104 is obtained, other types of chemical transformations can be carried out, such as to convert the X group of functionalized monomer 104 to a different X group. For example, in some embodiments, functionalized monomer 104 can be converted to a different monomer compound using further catalytic reactions, such as palladium cross-couplings, oxidation/reduction reactions, or cycloadditions, to name a few. Scheme 2 illustrates some representative method embodiments for such conversions using different X groups (e.g., Br or I). As shown in Scheme 2, vinyl halide monomers (200) can be converted to different monomer compounds comprising different X groups. With reference to Scheme 2, each Y independently can be selected from any Y group defined herein for Formula I. The present disclosure is not limited to those reactions disclosed in Scheme 2. For example, in some embodiments, further transformations (e.g., a Sonogashira reaction, such as for converting 200 to 206; a Negishi coupling, such as for converting 200 to 208; a Heck reaction, such as for converting 200 to 210; and a Suzuki coupling, such as for converting 200 to 212) can be carried out as discussed in more detail below.

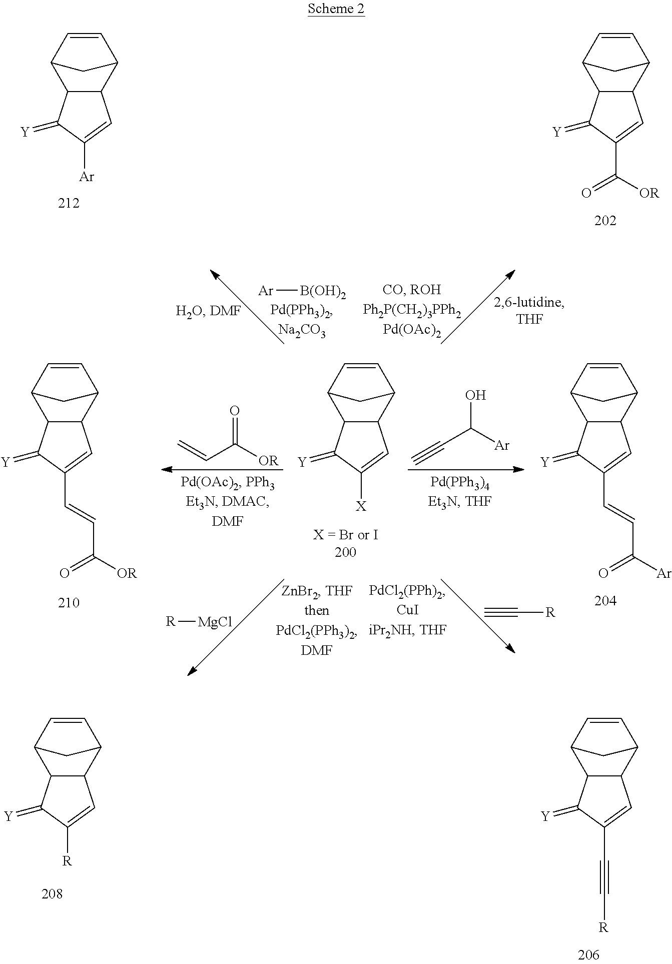

Particular reactions illustrated in Scheme 2 are discussed. In some embodiments, vinyl halide monomer 200 can be converted to ester compound 202 (wherein R can be aliphatic, haloaliphatic, haloheteroaliphatic, heteroaliphatic, aromatic, or an organic functional group) using conditions illustrated in Scheme 2. In other embodiments, vinyl halide monomer 200 can be converted to compound 204 (wherein “Ar” can be an aromatic ring system, such as an aryl or heteroaryl group, which can comprise one or more substituents that can be selected from aliphatic, haloaliphatic, haloheteroaliphatic, heteroaliphatic, aromatic, and/or organic functional groups) using conditions illustrated in Scheme 2. In yet additional embodiments, vinyl halide monomer 200 can be converted to alkyne 206 (wherein R can be hydrogen, aliphatic, haloaliphatic, haloheteroaliphatic, heteroaliphatic, aromatic, or an organic functional group) using conditions illustrated in Scheme 2. In yet further embodiments, vinyl halide monomer 200 can be converted to compound 208 (wherein R can be selected from aliphatic, haloaliphatic, haloheteroaliphatic, heteroaliphatic, aromatic, and/or organic functional groups) using conditions illustrated in Scheme 2. In some embodiments, vinyl halide monomer 200 can be converted to α,β-unsaturated ester compound 210 (wherein R can be selected from hydrogen, aliphatic, haloaliphatic, haloheteroaliphatic, heteroaliphatic, aromatic, and/or organic functional groups) using conditions illustrated in Scheme 2. In yet additional embodiments, vinyl halide monomer 200 can be converted to compound 212 (wherein “Ar” can be an aromatic ring system, such as an aryl or heteroaryl group, which can comprise one or more substituents that can be selected from aliphatic, haloaliphatic, haloheteroaliphatic, heteroaliphatic, aromatic, and/or organic functional groups) using conditions illustrated in Scheme 2. With reference to Scheme 2, the illustrated monomers can be the endo monomer or the exo monomer.

In some embodiments, a monomer having an X group that is a —CH₂OH group (which can be obtained by exposing compound 102 in Scheme 1 to a nucleophilic catalyst and formaldehyde, such as with Morita-Baylis-Hillman chemistry) can be converted to monomer embodiments having different X groups, such as oxidized X groups (e.g., aldehydes, esters, or carboxylic acids). Such transformations are summarized by Scheme 3 and can be accomplished using conditions recognized by a person of at least ordinary skill in the art with the benefit of the present disclosure. In some embodiments, monomer 300 can be converted to aldehyde 302 or to an ester (304, wherein R is aliphatic, haloaliphatic, haloheteroaliphatic, heteroaliphatic, aromatic, or an organic functional group) or to a carboxylic acid (304, wherein R is H). With reference to Scheme 3, the illustrated monomers can be the exo monomer or the endo monomer.

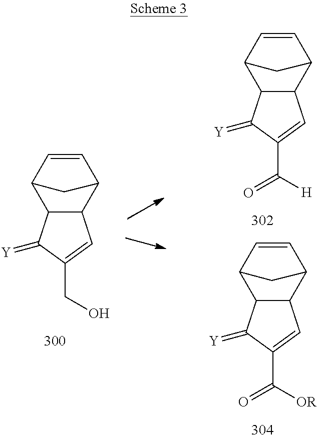

Monomer embodiments made according to any methods illustrated in Schemes 2 or 3 can also be modified to comprise yet other types of X groups by carrying out further chemical transformations. For example, in some embodiments, a compound 208 can be obtained wherein R is an alkene group, which can further be converted to a higher-order polycycle by reacting the diene-containing monomer with a suitable alkene-containing compound under appropriate conditions to facilitate a Diels-Alder reaction (e.g., conditions sufficient to promote a 4+2 cycloaddition) between the diene region of the monomer and the alkene, thereby providing a fused ring system. In some other examples, an aldehyde-containing monomer (e.g., compound 302) can be subjected to reductive amination conditions to provide an amine compound. Additional chemical moiety transformations are described herein, which can be conducted with the monomers pre- or post-polymerization and/or pre- or post-crosslinking. In any such embodiments, the monomers can be the endo monomer or the exo monomer.

In some embodiments, monomer embodiments comprising Y groups that can be selected from Y groups defined herein for Formula I can be made by converting a starting monomer comprising a Y group that is oxygen to the desired compound. Exemplary such embodiments are illustrated in Scheme 4, below.

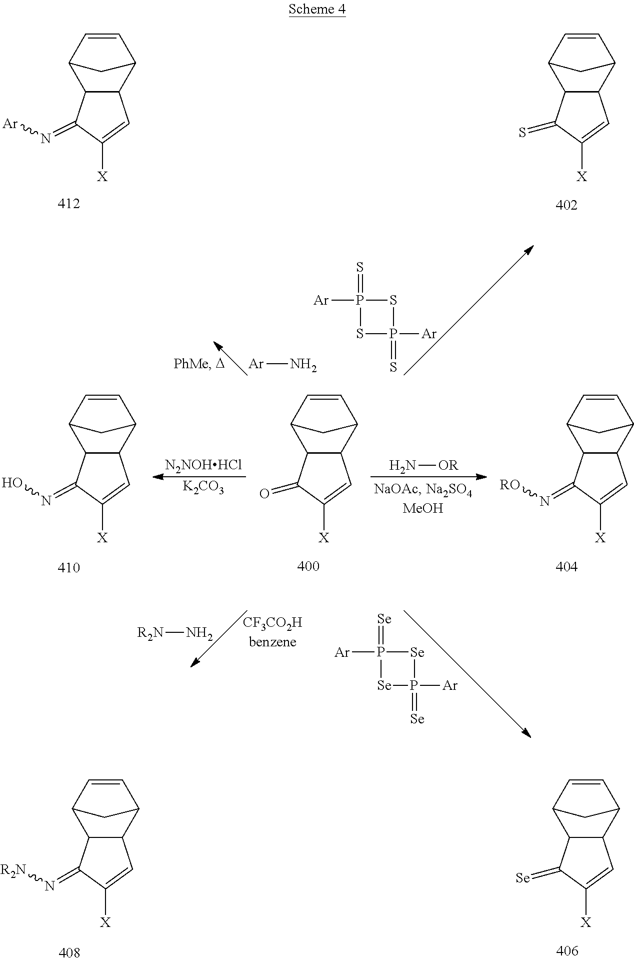

With reference to Scheme 4, ketone compound 400 can be converted to a thioketone compound 402 using the conditions illustrated in Scheme 4 (e.g., Lawesson's reagent). In other embodiments, ketone compound 400 can be converted to oxime-based compound 404 using the conditions illustrated in Scheme 4 (e.g., using an R-substituted hydroxyl amine compound and suitable reagents, and wherein, with reference to oxime-based compound 404, R can be aliphatic, haloaliphatic, haloheteroaliphatic, heteroaliphatic, aromatic, or an organic functional group). In yet additional embodiments, ketone compound 400 can be converted to selenoketone compound 406 using conditions illustrated in Scheme 406 (e.g., using Woollins' reagent). In yet additional embodiments, ketone compound 400 can be converted to hydrazone compound 408 using conditions illustrated in Scheme 4 (e.g., using a hydrazine-based reagent, and wherein, with reference to hydrazone compound 408, each R independently is hydrogen, aliphatic, haloaliphatic, haloheteroaliphatic, heteroaliphatic, aromatic, or an organic functional group). In some other embodiments, ketone compound 400 can be converted to oxime compound 410 using conditions illustrated in Scheme 4 (e.g., using hydroxyl amine and a base). In other embodiments, ketone compound 400 can be converted to imine compound 412 using conditions illustrated in Scheme 4 (e.g., an amine reagent, and wherein, with reference to imine compound 412, “Ar” can be an aromatic ring system, such as an aryl or heteroaryl group, which can comprise one or more substituents that can be selected from aliphatic, haloaliphatic, haloheteroaliphatic, heteroaliphatic, aromatic, and/or organic functional groups). With reference to Scheme 4, X of any of compounds 400 through 412 can be any X group as defined herein for Formula I and any of the illustrated monomers can be the endo monomer or the exo monomer.

Monomer embodiments of the present disclosure can be converted to polymers using a catalyst suitable for promoting ring-opening/metathesis reactions. Either of the endo monomer or the exo monomer (or a combination thereof) can be used. The catalyst may contain a ruthenium, molybdenum or tungsten metal, or may contain other metals. Exemplary catalysts include, but are not limited to, ruthenium-based metathesis catalysts, such as Grubbs catalysts (e.g., benzylidene-bis(tricyclohexylphosphino)-dichlororuthenium, CAS #172222-30-9; [1,3-bis-(2,4,6-trimethylphenyl)-2 imidazolidinylidene]dichloro(phenylmethylene)(tricyclohexylphosphino)ruthenium, CAS #246047-72-3, dichloro[1,3-bis(2,4,6-trimethylphenyl)-2-imidazolidinylidene](benzylidene)bis(3-bromopyridine)ruthenium, CAS #900169-53-1), Hoveyda-Grubbs catalysts (e.g., dichloro(o-isopropoxyphenylmethylene)(tricyclohexylphosphine)ruthenium(II), CAS #203714-71-0); dichloro(o-isopropoxyphenylmethylene)(tricyclohexylphosphine)ruthenium(II), CAS #203714-71-0; [1,3-Bis-(2,4,6-trimethylphenyl)-2-imidazolidinylidene]dichloro(o-isopropoxyphenylmethylene)ruthenium, CAS #301224-40-8), other Grubbs catalysts or similar catalysts (e.g., dichloro[1,3-bis(2-methylphenyl)-2-imidazolidinylidene](2-isopropoxyphenylmethylene)ruthenium(II), CAS #927429-61-6); dichloro[1,3-bis(2,6-isopropylphenyl)-2-imidazolidinylidene](2-isopropoxyphenylmethylene)ruthenium(II), CAS #635679-24-2); dichloro[1,3-bis(2,4,6-trimethylphenyl)-2-imidazolidinylidene](3-methyl-2-butenylidene) (tricyclohexylphosphine)ruthenium(II), CAS #253688-91-4; [2-(1-methylethoxy-O)phenylmethyl-C](nitrato-O,O′){rel-(2R,5R,7R)-adamantane-2,1-diyl[3-(2,4,6-trimethylphenyl)-1-imidazolidinyl-2-y lidene]}ruthenium, CAS #1352916-84-7, dichloro[1,3-bis(2,4,6-trimethylphenyl)-2-imidazolidinylidene][(5-isobutoxycarbonylamino)-(2-isopropoxy)benzylidene]ruthenium, CAS #1025728-57-7), Piers catalysts (e.g., dichloro(tricyclohexylphosphine)[(tricyclohexylphosphoranyl)methylidene]ruthenium(II) tetrafluoroborate, CAS #1020085-61-3); dichloro[1,3-bis(2,4,6-trimethylphenyl)-2-imidazolidinylidene][(tricyclohexylphosphoranyl)methylidene]ruthenium(II) tetrafluoroborate, CAS #832146-68-6); molybdenum carbine catalysts, such as catalysts having structures meeting the formula Cp₂TiCl₂/RMgX, wherein “Cp” is η⁵-cyclopentadienyl, R is CH₃, C₂H₅, i-C₃H₇, n-C₄H₉, n-C₆H₁₃, or C₆H₅, and X is Cl, Br, or I; ReCl₅/Me₄Sn catalyst systems; Tungsten(IV) or molybdenum(IV)-based catalysts, such as Schrock alkyldenes derived catalysts (e.g., W(C-t-Bu)(CH₂-t-Bu)₃, Mo(C-t-Bu)(CH₂-t-Bu)₃. W(CH-t-Bu)(O)(PR₃)₂Cl₂, W(CH-t-Bu)(O)(PR₃)Cl₂, Mo(NAr)(CH-t-Bu)(OHIPT)(Pyrrolide), and W(O)(CH-t-Bu)(OHMT)(Me₂Pyr)), Schrock-Hoveyda Catalyst (e.g., 2,6-Diisopropylphenylimido-neophylidene[(S)-(−)-BIPHEN]molybdenum(VI), CAS #205815-80-1); allyl silane/tungsten catalysts (e.g., catalysts consisting of WCl₆ and/or WOCl₄ and organosilicon compounds such as SiAllyl₄, SiMe₂Allyl₂, (iBu)₂Al—O—Al(iBu)₂), and the like. In some embodiments, the amount of catalyst is selected depending on the reaction rate desired. In some embodiments, 0.00001 to 0.10 mole of catalyst can be used per mole of monomer(s), such as 0.00005 to 0.001 mole of catalyst per mole of monomer(s).

In some particular embodiments, the polymerization step comprises using a ring opening metathesis polymerization (ROMP) mechanism to form the polymer. In yet further embodiments, the polymerization step comprises using a frontal ring opening metathesis polymerization (FROMP) mechanism to form the polymer. In such embodiments, the monomer is exposed to a catalyst and a thermally labile inhibitor (such as a phosphite compound), which facilitates maintaining the reaction mixture as a liquid. The reaction mixture is heated using an appropriate method known to those having at least ordinary skill in the art with the benefit of the present disclosure (e.g., applying an external heating element to the reaction vessel). Upon heating, a thermal wave is produced that propagates through the reaction mixture, which facilitates polymerization.

Scheme 5A summarizes an exemplary polymerization wherein ROMP takes place upon exposing monomer compound 500 to a catalyst as described above to provide polymer 502. In particular embodiments using a FROMP mechanism, the monomers that are used typically are the exo monomers; however, the endo monomers also could be used.

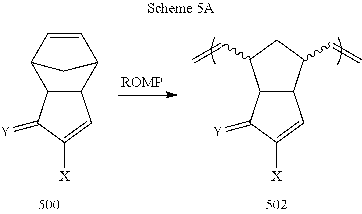

In yet other embodiments, metal-free conditions and/or catalysts may be used to promote the reaction. For example, the monomers can be polymerized in a suitable solvent, including polar solvents (such as THF, 2-MeTHF, DMSO, or other polar aprotic solvents known to those having at least ordinary skill in the art, with the benefit of the present disclosure) and non-polar solvents (such as CH₂Cl₂, toluene, or other hydrocarbon solvents like aliphatic-based solvents, such as hexane, pentane, heptane, hexanes, and the like) using an enol ether initiator (such as, ethyl propenyl ether, 1-methoxy-4-phenyl butane, or 2-cyclohexyl-1-methoxyethylene) and photoredox mediator (such as a pyrylium salt, an acridinium salt, a thiopyrylium salt, a persulfated salt, 2,4,6-tris(4-methoxyphenyl)pyrylium tetrafluoroborate or 2,3-dichloro-5,6-dicyano-1,4-benzoquinone) and further exposing the reaction mixture to blue LED light. In such embodiments, the conditions and reagents described by Goetz and Boydston, J. Am. Chem. Soc. 2015, 137, 7572-7575, which is incorporated herein by reference, can be adapted for use with the disclosed compounds to facilitate metal-free polymerization.

In some embodiments, the polymer can be crosslinked as described herein and as illustrated in Scheme 5B. In such embodiments, crosslinking can be carried out by exposing the polymer to heat. In some embodiments, the polymer is heated at a temperature ranging from greater than ambient temperature to 250° C. or higher, such as 70 to 250° C., 100° C. to 250° C. or higher, such as 100° C. to 200° C., or 110 to 200° C., or 110° C. to 150° C. In some additional embodiments, a chemical initiator, such as an anion or free radical or the like, may be added to stimulate and/or initiate crosslinking. In some embodiments, a crosslinked polymer can be obtained using a separate heating step after polymerization. In yet other embodiments, crosslinking may occur during polymerization.

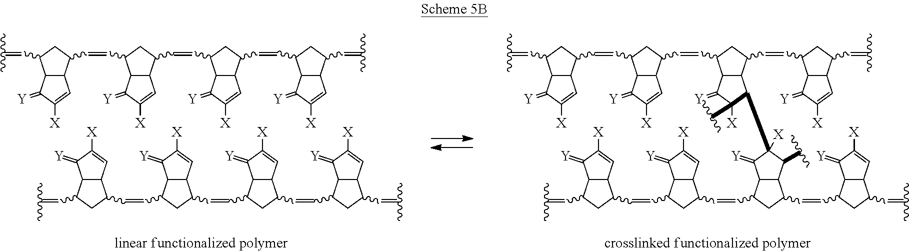

In particular embodiments, the crosslinking process is reversible. This is in contrast to unfunctionalized polydicyclopentadiene, where crosslinking (curing) is essentially irreversible. As a result, PDCPD is considered to be a thermoset material, and cannot be melted and reformed into new objects, which makes it non-recyclable for all practical purposes. When PDCPD objects reach the end of their lifespan they are either sent to landfills or else incinerated. By contrast, in particular polymer embodiments of the present disclosure, the crosslinking process is fundamentally reversible. In some embodiments, the degree of reversibility is defined by the overall ΔG for the reaction, as well as the free energy barrier ΔG^{‡}. These properties, in turn, can depend on the identity of the functional groups Y and X. As such, in some embodiments, reversibility of the crosslinking process can be controlled by selecting particular X and Y groups for the monomer subunits of the polymer(s) that are crosslinked. Solely by way of example, if X and Y are chosen in such a way as to make the crosslinking step substantially exergonic then there will be little to no reversibility under normal conditions. Such embodiments can comprise X and Y groups that are aliphatic. Alternatively, if X and Y are chosen in such a way as to make the crosslinking step closer to thermoneutral, the resulting equilibrium control of crosslinks within the material can be exploited to provide improved energy dissipation throughout the material. For example, when Y═O and X═CO₂R, the pendent substituted cyclopentene residues along the linear polymer chain encode a ketomethacrylate group, which can promote reversible crosslinking. These, and related functional groups, are known to polymerize through a head-to-tail olefin addition reaction that can be promoted by traces of moisture or sources of anions or free radicals. In the context of a functionalized polydicyclopentadiene, these head-to-tail polymerizations will constitute a dominant crosslinking mechanism. Energy dissipation of the polymer may be of particular interest for polymer embodiments that are used in ballistics applications, as discussed herein. In yet other embodiments, the crosslinking reaction may be chemically reversed by applying a particular stimulus (e.g., heat or a strong base, such as NaOH, KOH, K₂CO₃, amines, etc.). In some embodiments, such conditions can facilitate reverting the crosslinked material back to the linear polymer(s), at which point the polymers could be reformed into new objects. This type of thermoset recycling can be of particular interest for certain applications and/or to provide reusable materials.

In some further embodiments, the polymer can be reduced, or at least partially reduced, such that the double bonds formed from the ROMP or FROMP (or other polymerization method) between the monomers can be reduced to the saturated form(s), such as is illustrated in Scheme 6.

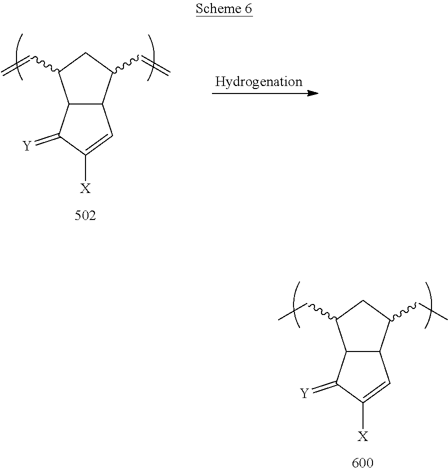

As illustrated in Scheme 6, a method for making reduced or partially reduced linear polymer 600 is disclosed, wherein polymer 502 is subjected to hydrogenation conditions. This method may involve hydrogenation over a palladium-, platinum-, or nickel-containing catalyst, or may involve hydrogenation (or transfer hydrogenation) over a different metal catalyst. Examples of such catalysts include, but are not limited to, palladium oxide, platinum oxide, Pd/C, Pt/C, tetrakis(triphenylphosphine)palladium(0), palladium acetate, nickel boride, and the like. Alternatively, metal-free conditions such as diimide reductions may be employed to promote reduction.

Polymer embodiments described herein (e.g., homopolymers, copolymers, and/or crosslinked forms thereof) can be further modified post-polymerization (and/or post-crosslinking) to provide functionalized polymer embodiments. Examples of such post-polymerization (and/or post-crosslinking) modifications are illustrated in Scheme 7. With reference to the “cargo” component illustrated in Scheme 7, this component can be selected from a chromogen (e.g., a dye), fluorophores, or active components (e.g., antibacterial or antifungal agents, peptides, and the like) and may be connected directly to the reactive group used for conjugation (e.g., the oxygen atom of oxime-based compound 702, the nitrogen atom of the amide of compound 708, or the oxygen atom of the carboxyl group of compound 710). Alternatively, the molecular cargo may be connected to the reactive group through a suitable linker group, such as a bifunctional linker comprising terminal functional groups that facilitate coupling the linker group to the cargo moiety and the polymer (e.g., amine groups, a hydroxyl group, carbonyl-containing groups, amide-containing groups, azide containing groups, alkyne groups, and the like). Bifunctional groups can further comprise an aliphatic, haloaliphatic, haloheteroaliphatic, heteroaliphatic, aromatic, or organic functional groups that are positioned between the terminal functional groups. With reference to Scheme 7, each X and each Y independently can be selected from the X and Y groups defined for Formula I, herein.

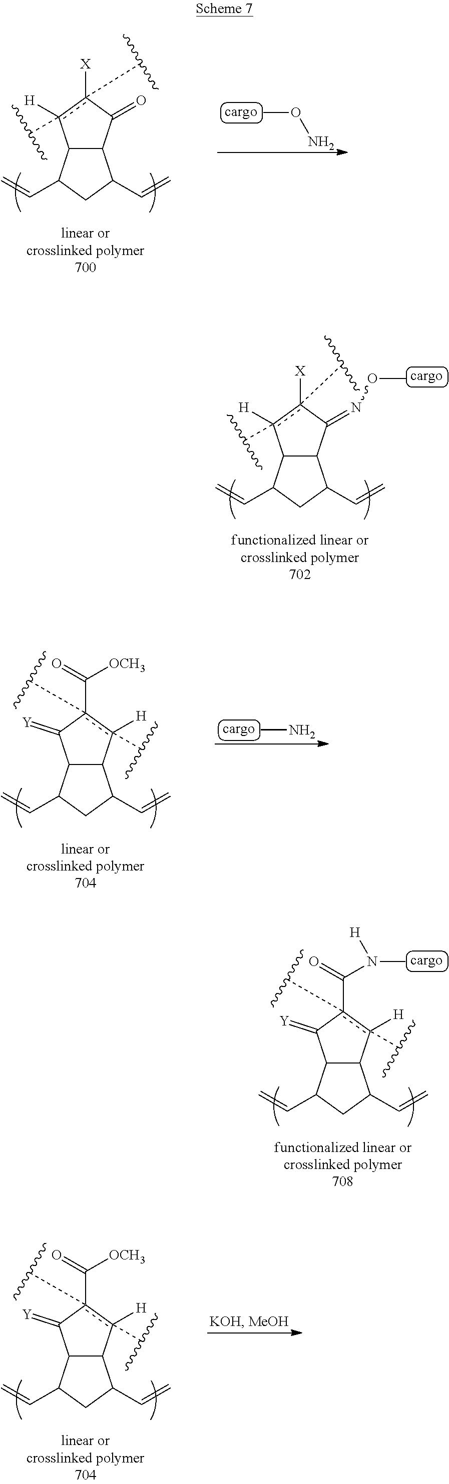

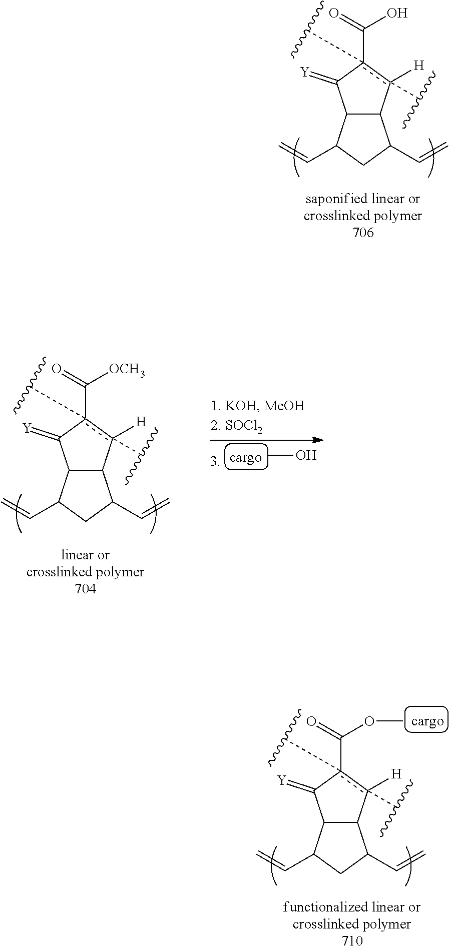

In yet further embodiments, polymer embodiments described herein (e.g., homopolymers, copolymers, and/or crosslinked forms thereof) can be further modified post-polymerization (and/or post-crosslinking) to provide functionalized polymer embodiments using methods such as those illustrated in Scheme 8. As described above, the “cargo” component of compounds shown in Scheme 8 can be selected from a chromogen (e.g., a dye), fluorophores, or active components (e.g., antibacterial or antifungal agents, peptides, and the like) and may be connected directly to the reactive group used for conjugation (e.g., the carbon atom of ketone compound 802, the nitrogen atom of triazine compound 806, or β-carbon of ester compound 810). Alternatively, the molecular cargo may be connected to the reactive group through a suitable linker group, such as a bifunctional linker comprising terminal functional groups that facilitate coupling the linker group to the cargo moiety and the polymer (e.g., amine groups, a hydroxyl group, carbonyl-containing groups, amide-containing groups, azide-containing groups, alkyne groups, and the like). Bifunctional groups can further comprise an aliphatic, haloaliphatic, haloheteroaliphatic, heteroaliphatic, aromatic, or organic functional groups that are positioned between the terminal functional groups. With reference to Scheme 8, each Y independently can be selected from the Y groups defined for Formula I, herein.

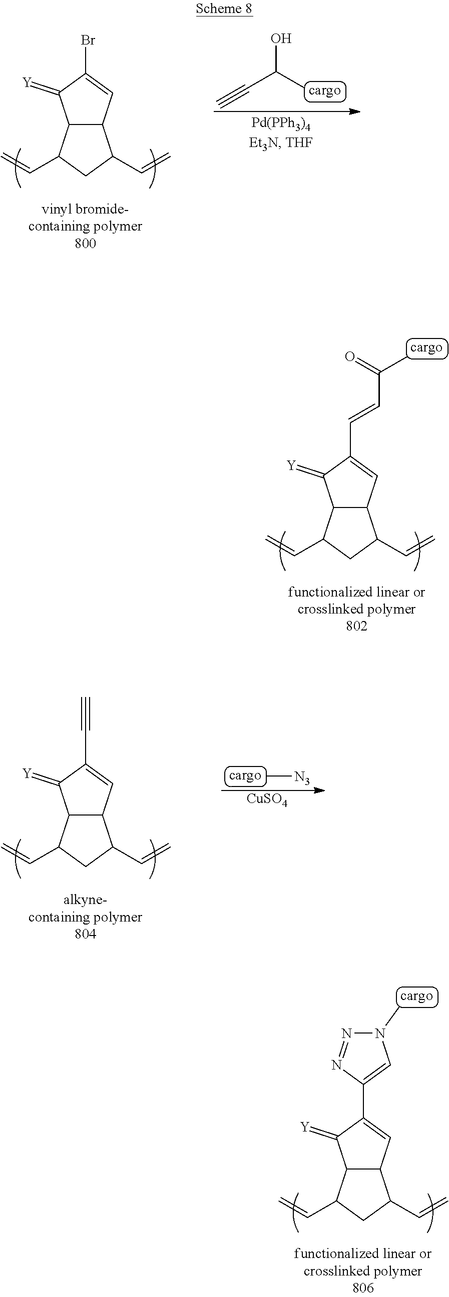

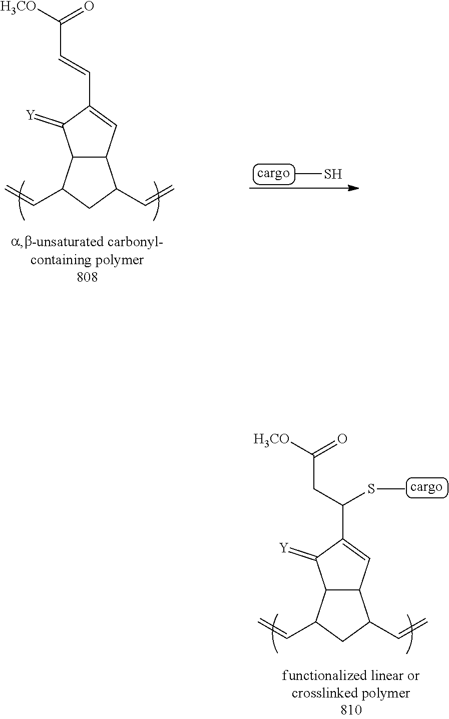

In some additional embodiments, other types of chemical moiety transformations can be carried out after polymerization. Without intending to be limiting examples, certain embodiments of further chemical moiety transformations are illustrated in Scheme 9. For example, in some embodiments, a polymer comprising an alkyne moiety (e.g., compound 900) can be reduced under hydrogenation conditions to provide the corresponding polymer comprising a saturated side-chain (compound 902). In some other embodiments, a ketone-containing compound 904 can be reduced to the corresponding alcohol compound 906 using a reducing agent (e.g., LiAlH₄). In yet additional embodiments, the polymer can comprise a diene motif, such as is illustrated in compound 908 and this can be reacted with an alkene under conditions suitable to promote a Diels-Alder reaction between the diene motif of the monomer and the alkene, thereby providing polymer 910, which comprises a fused ring system. Additional conditions that can facilitate transforming a chemical moiety of the polymer to a different chemical moiety include, but are not limited to, reductive amination conditions, oxime-forming conditions, saponification conditions, amide bond forming conditions, transesterification conditions, palladium-mediated cross-coupling conditions, click chemistry conditions, conjugate addition conditions, carbonyl-reducing conditions, hydrogenation conditions, or combinations thereof.

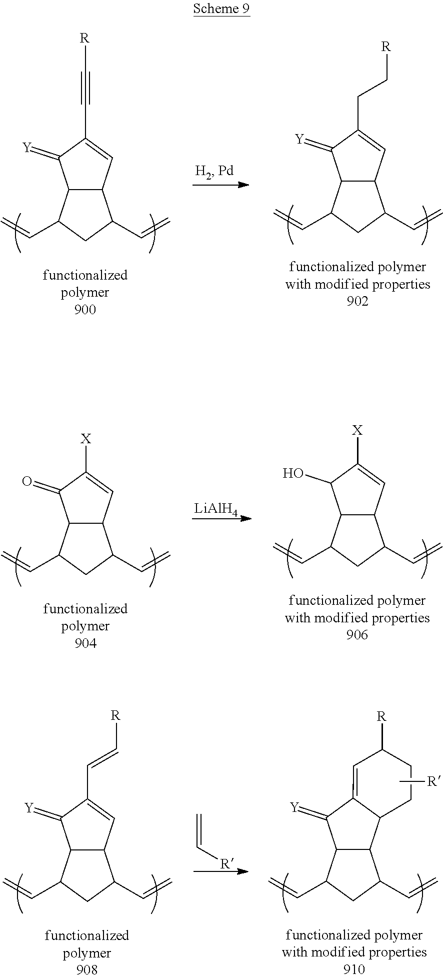

### V. Method of Use

Yet another aspect of this disclosure relates to using the monomer and/or polymer embodiments of the present disclosure for manufacturing processes, including injection molding, resin transfer molding, reaction injection molding (RIM), sheet molding compound (SMC) processes, bulk molding compound (BMC) processes, glass reinforced plastic (GRP) processes, and other processes used in polymer manufacturing. In yet additional embodiments, polymer embodiments of the present disclosure (including linear homo- or copolymers and/or crosslinked homo- or copolymers) can be used in, or can form, foams, gels, aerogels, films, coatings, or composite materials (including, but not limited to, fiberglass and carbon fiber composites). In particular embodiments, polymer and/or copolymer embodiment disclosed herein can be used to make composite materials suitable in ballistic applications. For example, polymer-based armor embodiments comprising compound/polymer embodiments are disclosed herein. These materials offer an attractive alternative to steel armor, since organic polymers are lighter than steel, and have a large elastic deformation response. Also, given their low density and high strength (which can be further increased when coupled with a suitable fiber-reinforcing agent), polymer embodiments disclosed herein can be used in maritime craft, helicopters, and aircraft.

### VI. Overview of Several Embodiments

Disclosed herein are embodiments of a compound having a structure according to Formula I

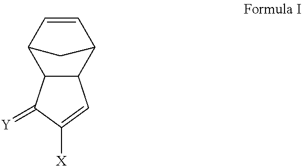

wherein
X is selected from hydrogen, deuterium, halogen, aliphatic, haloaliphatic, haloheteroaliphatic, heteroaliphatic, aromatic, an organic functional group, a detectable moiety, or an active component, or any combination of such groups; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, or an organic functional group; provided that if Y is O, then X is not hydrogen.

In any or all embodiments, X is hydrogen, deuterium, Cl, Br, I, alkyl, alkynyl, alkenyl, heteroalkyl, heteroalkenyl, heteroalkynyl, aryl, heteroaryl, hydroxyl, aldehyde, ester, amide, ketone, a fluorophore, an active component, or a combination thereof.

In any or all of the above embodiments, X is hydrogen; deuterium; Br; I; lower alkyl; —C═CR^{d}, wherein R^{d} is selected from hydrogen, aliphatic, haloaliphatic, haloheteroaliphatic, heteroaliphatic, aromatic, or an organic functional group; —C═CR^{d}R^{e}, wherein R^{d} and R^{e} independently are selected from hydrogen, aliphatic, haloaliphatic, haloheteroaliphatic, heteroaliphatic, aromatic, or an organic functional group; thioether; ether; amine; hydroxyl; aldehyde; ester; amide; ketone; an aryl ring system comprising from 6 ring atoms to 10 ring atoms; a heteroaryl ring system comprising from 5 ring atoms to 10 ring atoms, with at least one heteroatom; fluorescein; rhodamine; BODIPY; an antibacterial agent; an antifungal agent; an anticancer agent; a peptide promoting cellular adhesion; a signaling factor controlling cellular growth or motility; a vitamin; a cofactor; or any combination thereof.

In any or all of the above embodiments, Y is O; S; N-aromatic; or N—OR^{d} or N—NR^{d}R^{e}, wherein R^{d} and R^{e} independently are selected from hydrogen, alkyl, heteroalkyl, heteroalkenyl, heteroalkynyl, aryl, or heteroaryl.

In any or all of the above embodiments, Y is O, S, Se, N-phenyl, N-pyridinyl, N—OH, N—OMe, N—OEt, N—NH₂, N—NHMe, N—N(Me)₂, N—N(Et)₂, or N—N(Me)Et.

In any or all of the above embodiments, the compound is selected from any of compounds 2-86, 2a-86a, and/or 2b-86b as disclosed herein and wherein R^{d} and R^{e} for any of compounds 2-86, 2a-86a, and/or 2b-86b are independently selected from hydrogen, aliphatic, haloaliphatic, haloheteroaliphatic, heteroaliphatic, aromatic, or an organic functional group; each n independently is an integer selected from 0 to 25; and each m independently is an integer selected from 0 to 5.

In any or all of the above embodiments, the compound is an endo isomer.

In any or all of the above embodiments, the compound is an exo isomer.

Also disclosed herein are embodiments of a polymer, comprising at least one monomer subunit having a structure according to Formula IA

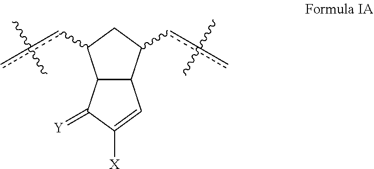

wherein
X is selected from hydrogen, deuterium, halogen, aliphatic, haloaliphatic, haloheteroaliphatic, heteroaliphatic, aromatic, an organic functional group, a detectable moiety, or an active component, or any combination thereof; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, an organic functional group, a detectable moiety, an active component, or a combination thereof.

In any or all of the above embodiments, the polymer is a homopolymer having a structure according to Formula II

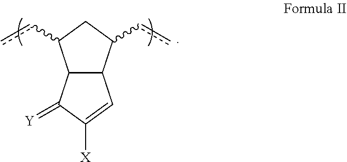

In any or all of the above embodiments, the polymer is a copolymer having a structure according to Formula IIIA or IIIB

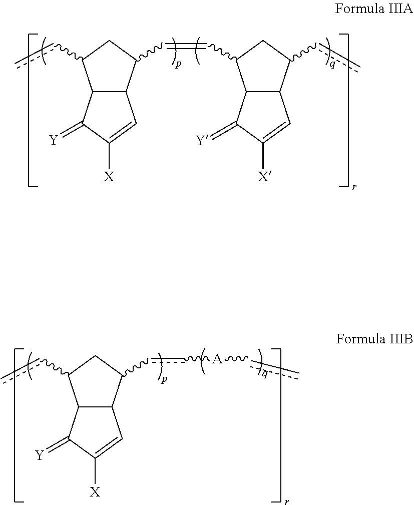

wherein each X and each X′ independently for each occurrence is selected from hydrogen, deuterium, halogen, aliphatic, haloaliphatic, haloheteroaliphatic, heteroaliphatic, aromatic, an organic functional group, a detectable moiety, or an active component, or any combination thereof; each Y and each Y independently for each occurrence is selected from O; S; Se; or NR^{c}, wherein R^{c} is hydrogen, —OR^{d}, or —NR^{d}R^{e}, wherein R^{d} and R^{c} independently are selected from hydrogen, aliphatic, haloaliphatic, haloheteroaliphatic, heteroaliphatic, aromatic, an organic functional group a detectable moiety, an active component, or a combination thereof; A is an aliphatic group; each of p, q, and r independently is an integer of at least 1; and provided that either (i) X and X′ are different, or (ii) Y and Y′ are different.

In any or all of the above embodiments, A is an acyclic aliphatic group or a cyclic aliphatic group.

In any or all of the above embodiments, the polymer is crosslinked with a second polymer.

In any or all of the above embodiments, the polymer comprises an end-capping group selected from hydrogen, aromatic, aliphatic, haloaliphatic, haloheteroaliphatic, heteroaliphatic, or an organic functional group.

Also disclosed herein are embodiments of a method, comprising:
forming a polymer by combining (i) a catalyst comprising ruthenium (Ru), molybdenum (Mo), tungsten (W), titanium (Ti), tantalum (Ta), or a combination thereof; and (ii) a compound having a Formula I

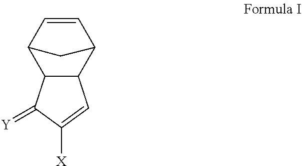

wherein
X is selected from hydrogen, deuterium, halogen, aliphatic, haloaliphatic, haloheteroaliphatic, heteroaliphatic, aromatic, an organic functional group, a detectable moiety, or an active component, or any combination of such groups; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, or an organic functional group.

In any or all of the above embodiments, the method of claim 15, further comprising exposing the polymer to conditions sufficient to promote crosslinking.

In any or all of the above embodiments, the conditions sufficient to promote crosslinking include exposing the polymer to air, radical crosslinking conditions, anionic crosslinking conditions, and/or a crosslinking reagent.

In any or all of the above embodiments, the method further comprises exposing the polymer to hydrogenation conditions to provide a polymer comprising saturated bonds between monomer subunits.

In any or all of the above embodiments, the method further comprises exposing the polymer to conditions capable of transforming a chemical moiety of the polymer to a different chemical moiety.

In any or all of the above embodiments, the conditions are selected from reductive amination conditions, 4+2 cycloaddition conditions, oxime-forming conditions, saponification conditions, amide bond forming conditions, transesterification conditions, palladium-mediated cross-coupling conditions, click chemistry conditions, conjugate addition conditions, carbonyl-reducing conditions, hydrogenation conditions, or combinations thereof.

Also disclosed herein are embodiments of a composite material, comprising: a strikeface layer; a polymer layer that is physically associated with the strikeface layer and that comprises a polymer according to any one of claims 9-14; a reinforcing fiber layer that is physically associated with the polymer layer.

In any or all such embodiments, the strikeface layer is made of a ceramic or steel material, the polymer layer comprises an oxaPDCPD polymer, or an oxaPDCPD-co-PDCPD co-polymer, and the reinforcing fiber layer is made of a glass, carbon, or ultra-high-molecular-weight polyethylene.

### VII. Examples

### Example 1

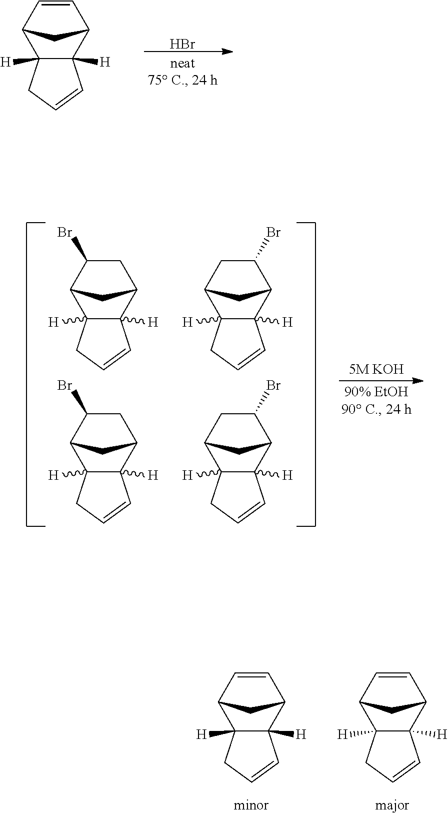

This example describes a method for making a monomer starting material as described herein, which can be used to make functionalized monomers of Formula I. Hydrobromic acid (48%, 130 mL) and dicyclopentadiene (49.98 g, 0.378 mol) were stirred at 75° C. for 24 hours under an argon atmosphere. The reaction was diluted with water and extracted with ether. The extracts were washed with saturated sodium bicarbonate and dried over sodium sulfate. The organic layer containing the product was concentrated in vacuo. The crude dicyclopentadiene hydrobromide adduct was carried forward without purification.

The dicyclopentadiene hydrobromide adduct (85 g) and potassium hydroxide solution (5 M in 95% ethanol, 260 mL) were heated to reflux under argon with vigorous stirring for 24 hours. The reaction was diluted with water and extracted with ether. The extracts were washed with water and dried over sodium sulfate. The organic layer containing the product was concentrated in vacuo. Distillation yielded a clear colorless liquid. ¹H NMR (300 MHZ, CDCl₃) δ 6.06 (dt, J=8.4, 5.6 Hz, 2H), 5.77-5.51 (m, 2H), 2.67 (d, J=5.6 Hz, 1H), 2.57 (s, 1H), 2.54-2.45 (m, 2H), 2.23 (t, J=8.6 Hz, 1H), 1.92-1.81 (m, 1H), 1.47 (d, J=8.6 Hz, 1H), 1.30 (dd, J=8.5, 1.4 Hz, 1H).

### Example 2

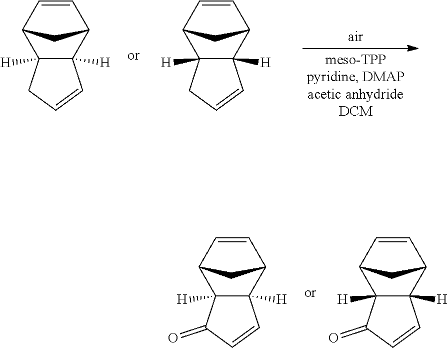 [SMILES: C1=C[C@H]2[C@@H](C1)[C@@H]1C=C[C@H]2C1 ; C1=C[C@@H]2[C@H](C1)[C@@H]1C=C[C@H]2C1 ; O=C1C=C[C@H]2[C@@H]1[C@@H]1C=C[C@H]2C1 ; O=C1C=C[C@@H]2[C@H]1[C@@H]1C=C[C@H]2C1]

In this example, a ketone-containing monomer was made.

Small-Scale Synthesis: meso-Tetraphenylporphyrin (meso-TPP; 13.2 mg; 0.021 mmol) and DMAP (224.8 mg; 1.840 mmol) were dissolved in dichloromethane (100 mL). Acetic anhydride (8.5 mL), pyridine (3 mL) and endo or exo-DCPD (10 mL; 74.5 mmol) were subsequently added. The mixture was irradiated with two halogen bulbs and sparged with air for 192 hours. The reaction mixture was diluted with dichloromethane and washed with 2 M HCl, brine, then saturated sodium bicarbonate. The organic layer containing the product was concentrated in vacuo to afford a black oil. endo-Dicyclopentadienone was purified via reverse recrystallization in pentanes, yielding white crystals. exo-Dicyclopentadienone was purified via vacuum distillation yielding a clear liquid.

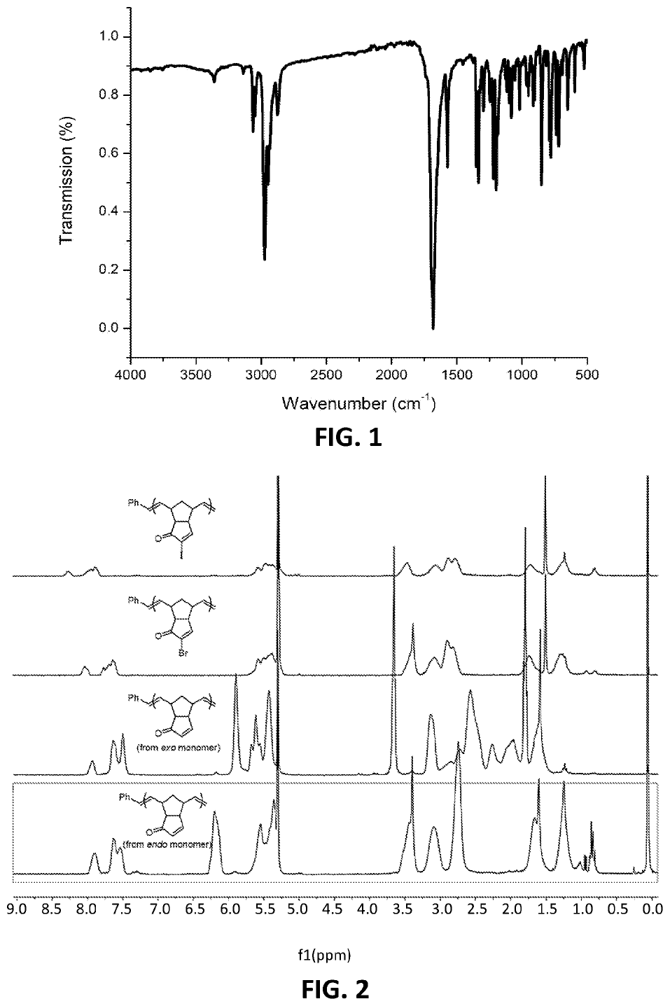

endo-Dicyclopentadienone: ¹H NMR (300 MHZ, CDCl₃) δ 7.38 (dd, J=5.7, 2.5 Hz, 1H), 5.98-5.76 (m, 3H), 3.42 (dddd, J=5.6, 4.2, 2.8, 1.5 Hz, 1H), 3.22 (s, 1H), 2.97 (ddt, J=4.3, 2.9, 1.4 Hz, 1H), 2.84-2.78 (m, 1H), 1.76 (dt, J=8.4, 1.7 Hz, 1H), 1.65-1.59 (m, 1H). MP (DSC) 50° C. See FIG. 1 for AT IR spectrum.

exo-Dicyclopentadienone: ¹H NMR (300 MHZ, CDCl₃) δ 7.56 (dt, J=5.6, 2.1 Hz, 1H), 6.25 (dddq, J=15.5, 7.9, 5.3, 2.3 Hz, 3H), 2.92 (s, 1H), 2.88-2.83 (m, 1H), 2.71 (d, J=2.9 Hz, 1H), 2.30-2.24 (m, 1H), 1.40 (dq, J=9.6, 1.6 Hz, 1H), 1.29 (dt, J=9.4, 1.6 Hz, 1H).

Large-Scale Synthesis: 171 mg (0.28 mmol) meso-TPP and 5.5812 (45.68 mmol) DMAP were dissolved in 2.2 L of DCM. 230 mL (2.43 mol) acetic anhydride, 94 mL (1.16 mol) of pyridine and 300.36 g (2.27 mol) DCPD were subsequently added. The mixture was irradiated in a 5 L photoreactor using a Boryli BTL P28S 500W halogen bulb contained in a cold finger. The mixture was sparged with air and reacted for 27 days. The reaction mixture was washed with 2 M HCl, then saturated sodium bicarbonate. The aqueous extracts were back extracted with DCM. All DCM factions were combined and concentrated in vacuo yielding a black oil. The crude reaction mixture was adhered to a silica gel plug and washed with hexanes and eluted with 3:1 hexanes to ethyl acetate. The resulting concentrate was then purified via vacuum distillation yielding 167.54 g (50.44% yield) of oxaDCPD as a white crystal.

1H NMR (300 MHZ, Chloroform-d) δ 7.38 (dd, J=5.8, 2.6 Hz, 1H), 5.98-5.75 (m, 3H), 3.45-3.39 (m, 1H), 3.22 (s, 1H), 2.97 (dq, J=2.9, 1.4 Hz, 1H), 2.83-2.78 (m, 1H), 1.76 (dd, J=8.4, 1.7 Hz, 1H), 1.62 (d, J=8.5 Hz, 1H).

### Example 3

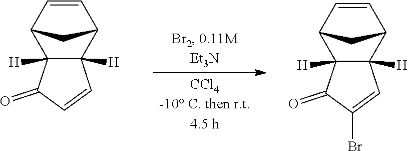 [SMILES: O=C1C=C[C@@H]2[C@H]1[C@@H]1C=C[C@H]2C1 ; O=C1C(Br)=C[C@@H]2[C@H]1[C@@H]1C=C[C@H]2C1]

In this example, a bromine-containing, functionalized monomer embodiment falling within Formula I described herein was made. endo-Dicyclopentadienone (500 mg; 3.42 mol) in dry, degassed CCl₄ (13 mL) was rapidly injected to 34 mL of 0.11 M bromine in dry CCl₄ under argon, in a flask chilled to −10° C. Triethylamine (2.9 M; 4.5 mL) was added immediately afterword. After 30 minutes, the cold bath was removed. After 4 hours, 25 mL of saturated sodium thiosulfate was injected, and the mixture was extracted with ether. The extracts were washed with water and dried over sodium sulfate, after which and the solvent was removed in vacuo. Column chromatography with 9:1 hexanes:ethyl acetate afforded the desired product as white crystals. ¹H NMR (300 MHZ, CDCl₃) δ 7.50 (d, J=2.9 Hz, 1H), 5.89 (ddd, J=32.7, 5.7, 2.9 Hz, 2H), 3.37-3.27 (m, 2H), 3.02 (pd, J=2.8, 1.6 Hz, 1H), 2.91 (t, J=5.1 Hz, 1H), 1.78 (dt, J=8.7, 1.8 Hz, 1H), 1.61 (d, J=8.6 Hz, 2H).

### Example 4

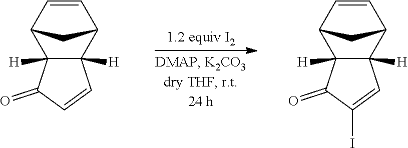 [SMILES: O=C1C=C[C@@H]2[C@H]1[C@@H]1C=C[C@H]2C1 ; O=C1C(I)=C[C@@H]2[C@H]1[C@@H]1C=C[C@H]2C1]

In this example, an iodine-containing functionalized monomer embodiment falling within Formula I described herein was made. endo-Dicyclopentadienone (300 mg; 2.05 mmol), 12 (615 mg; 2.42 mmol), DMAP (61.5 mg; 0.503 mmol), and K₂CO₃ (1.2498 g) were stirred in 16.5 mL dry THF under argon for 24 hours. The reaction was quenched with 10 mL saturated sodium thiosulfate and extracted with ethyl acetate. The extracts were washed with brine and dried over sodium sulfate. The organic layer containing the product was concentrated in vacuo. Recrystallization in pentane yielded white crystals. ¹H NMR (300 MHz, CDCl₃) δ 7.73 (d, J=2.8 Hz, 1H), 5.95-5.82 (m, 2H), 3.43 (d, J=3.9 Hz, 1H), 3.28 (s, 1H), 3.03 (s, 1H), 2.89 (t, J=5.1 Hz, 1H), 1.76 (d, J=8.7 Hz, 1H), 1.62 (d, J=8.6 Hz, 1H).

### Example 5

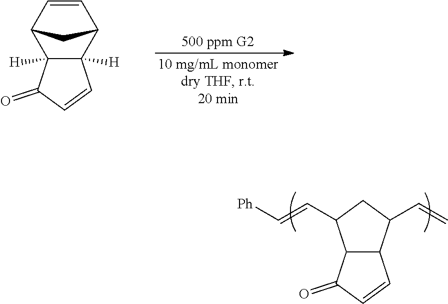

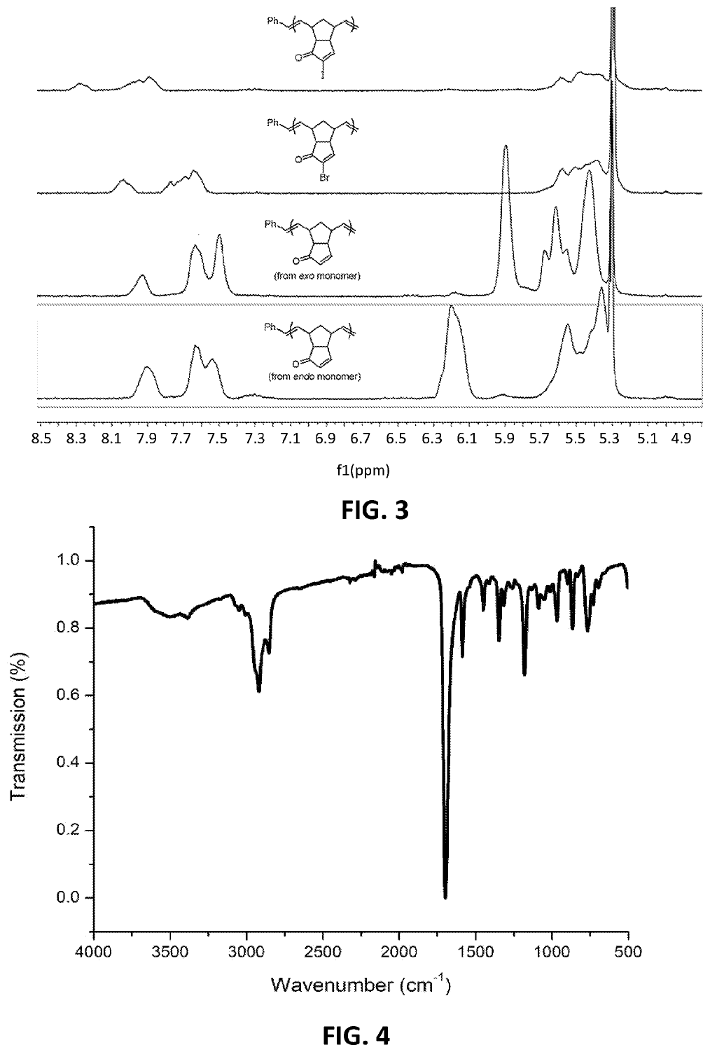

In this example, a ketone exo-monomer was polymerized to form a homopolymer made up of repeating subunits of the monomer embodiment. A flame dried flask was charged with Grubbs 2^{nd} generation catalyst (4.2 mg; 0.0049 mmol) and placed under an argon atmosphere. Dry THF (10 mL) was added to dissolve the catalyst. exo-Dicyclopentadienone (108.8 mg; 0.7448 mmol) was added by syringe, and the reaction mixture was vigorously stirred. After 20 minutes, 1 mL of ethyl vinyl ether was injected, and the reaction was stirred for 10 more minutes. The resulting polymer was precipitated with methanol and isolated via centrifugation. The crude isolated polymer was purified by two subsequent rounds of precipitation from dichloromethane and methanol, isolating the product in each case by centrifugation. 1H NMR (300 MHZ, CD₂Cl₂) δ 8.00-7.45 (m, 1H), 5.95-5.83 (m, 1H), 5.71-5.34 (m, 2H), 3.66 (t, J=6.1 Hz, 1H), 3.13 (s, 1H), 2.58 (s, 2H), 2.12 (d, J=84.1 Hz, 1H). See FIGS. 2 and 3 for the spectrum at ppm values ranging from 0-9 ppm and 4.8-8.5 ppm, respectively.

### Example 6

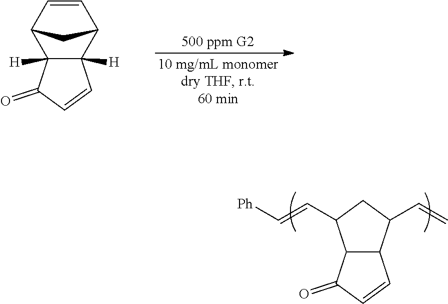

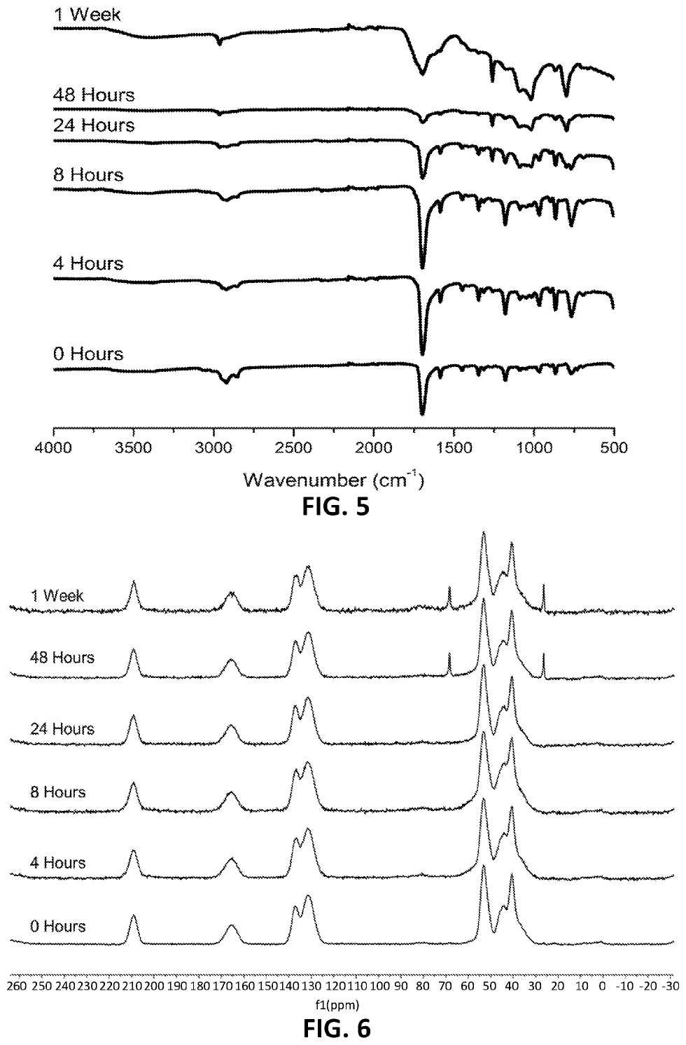

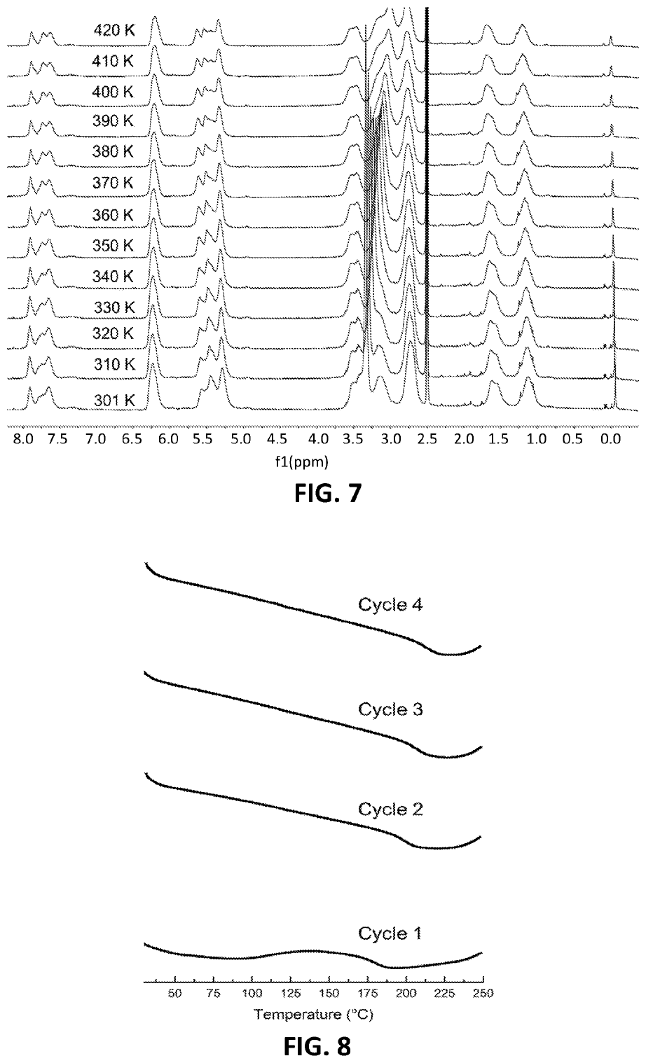

In this example, a ketone endo-monomer was polymerized to form a homopolymer made up of repeating subunits of the monomer embodiment. A solution of Grubbs 2^{nd} generation catalyst (500 ppm) in dry THF (50 mL) was added to endo-dicyclopentadienone (513.2 mg; 3.513 mol) in a flame dried reaction vessel under an atmosphere of argon. The reaction was stirred vigorously. After 60 minutes, 5 mL of ethyl vinyl ether was injected, and the reaction was stirred for 10 more minutes. The resulting polymer was precipitated with methanol and isolated via centrifugation. The crude isolated polymer was purified by two subsequent rounds of precipitation from dichloromethane and methanol, isolating the product in each case by centrifugation. ¹H NMR (300 MHZ, CD₂Cl₂) δ 7.98-7.44 (m, 1H), 6.20 (s, 1H), 5.68-5.31 (m, 2H), 3.40 (s, 1H), 3.09 (s, 1H), 2.75 (s, 2H), 1.25 (s, 1H). See FIGS. 2 and 3 for the spectrum at ppm values ranging from 0-9 ppm and 4.8-8.5 ppm, respectively. ¹³C NMR (126 MHZ, Methylene Chloride-d) o 209.98, 136.82, 54.43, 54.22, 54.00, 53.78, 53.57, 40.77, 40.49, 30.22. See FIG. 4 for AT IR spectrum of the linear polymer and FIG. 5 for the AT IR spectrum of progressively crosslinked poly-endo-dicyclopentadienone. FIGS. 6 and 7 show the solid state 13C NMR spectrum and variable temperature ¹H NMR spectrum, respectively, of progressively crosslinked poly-endo-dicyclopentadienone. FIG. 8 shows representative sequential DSC traces of poly-endo-dicyclopentadienone.

### Example 7

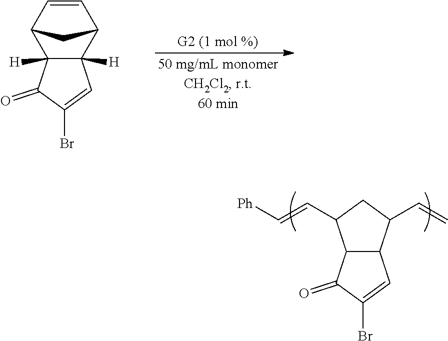

In this example, a bromine-containing, functionalized monomer embodiment was polymerized to form a homopolymer made up of repeating subunits of the monomer embodiment. endo-2-Bromodicyclopentadienone (97.4 mg; 0.433 mmol) was dissolved in dry dichloromethane (1 mL) and injected into a flame dried reaction vessel containing a solution of Grubbs 2nd generation catalyst (4.2 mg; 0.0049 mmol) in dry dichloromethane (1 mL), under an atmosphere of argon. After 60 minutes, 5 mL of ethyl vinyl ether was injected, and the reaction was stirred for 10 more minutes. The resulting polymer was precipitated with methanol and isolated via centrifugation. The crude isolated polymer was purified by two subsequent rounds of precipitation from dichloromethane and methanol, isolating the product in each case by centrifugation. ¹H NMR (300 MHZ, CD₂Cl₂) δ 8.11-7.56 (m, 1H), 5.71-5.33 (m, 2H), 3.39 (s, 1H), 3.25-2.68 (m, 3H), 1.74 (s, 1H), 1.41-1.15 (m, 1H). See FIGS. 2 and 3 for the spectrum at ppm values ranging from 0-9 ppm and 4.8-8.5 ppm, respectively.

### Example 8

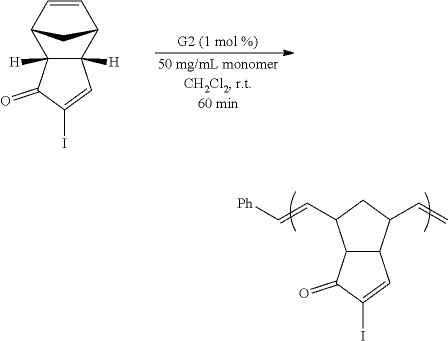

In this example, an iodine-containing, functionalized monomer embodiment was polymerized to form a homopolymer made up of repeating subunits of the monomer embodiment. endo-2-Iododicyclopentadienone (104.7 mg (0.385 mmol) was dissolved in dry dichloromethane (1 mL) and injected into a flame dried reaction vessel containing a solution of Grubbs 2nd generation catalyst (4.0 mg; 0.0047 mmol) in dry dichloromethane (1 mL), under an atmosphere of argon. After 60 minutes, 5 mL of ethyl vinyl ether was injected, and the reaction was stirred for 10 more minutes. The resulting polymer was precipitated with methanol and isolated via centrifugation. The crude isolated polymer was purified by two subsequent rounds of precipitation from dichloromethane and methanol, isolating the product in each case by centrifugation. ¹H NMR (300 MHZ, CD₂Cl₂) δ 8.34-7.82 (m, 1H), 5.68-5.33 (m, 2H), 3.48 (s, 1H), 3.07 (s, 1H), 2.84 (d, J=31.8 Hz, 2H), 1.72 (s, 1H), 1.24 (s, 1H). See FIGS. 2 and 3 for the spectrum at ppm values ranging from 0-9 ppm and 4.8-8.5 ppm, respectively.

### Example 9

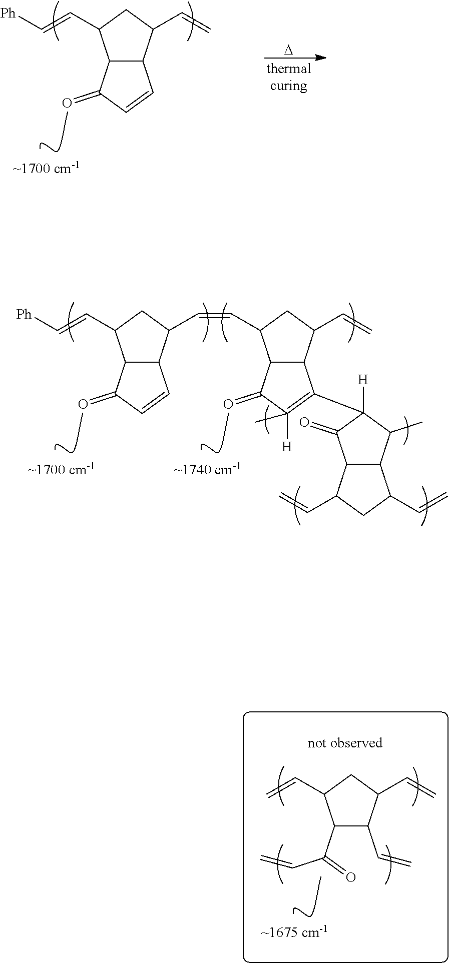

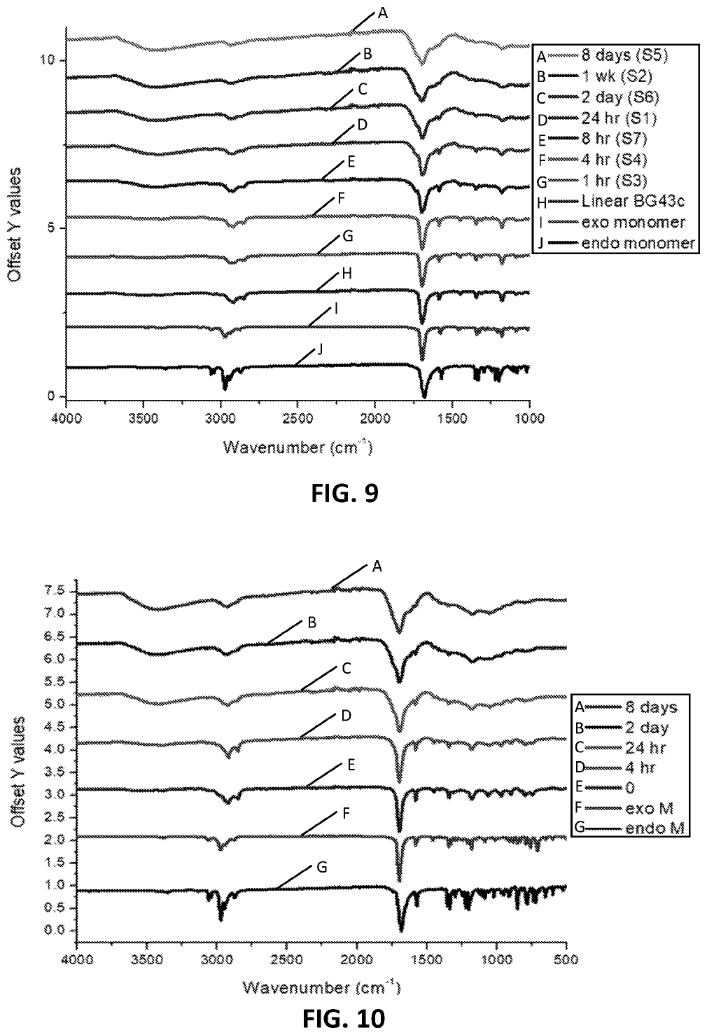

In this example, a polymer embodiment prepared from endo-dicyclopentadienone was cured to provide a crosslinked polymer. Samples of freshly prepared linear oxaPDCPD were thermally cured at 110° C. for 8 days. Individual samples were withdrawn at regular intervals and assessed by infrared spectroscopy to determine the extent of crosslinking and the type of chemical crosslink present within the material. Within the first 24 hours, a second carbonyl stretch was observed at higher wavenumbers than the carbonyl stretch for the linear polymer. These data (see FIG. 9) are consistent with a crosslinking mechanism in which the enone motif within the pendent cyclopentenone residues undergo olefin-addition polymerization. The data are inconsistent with the formation of crosslinks through secondary metathesis events.

### Example 10

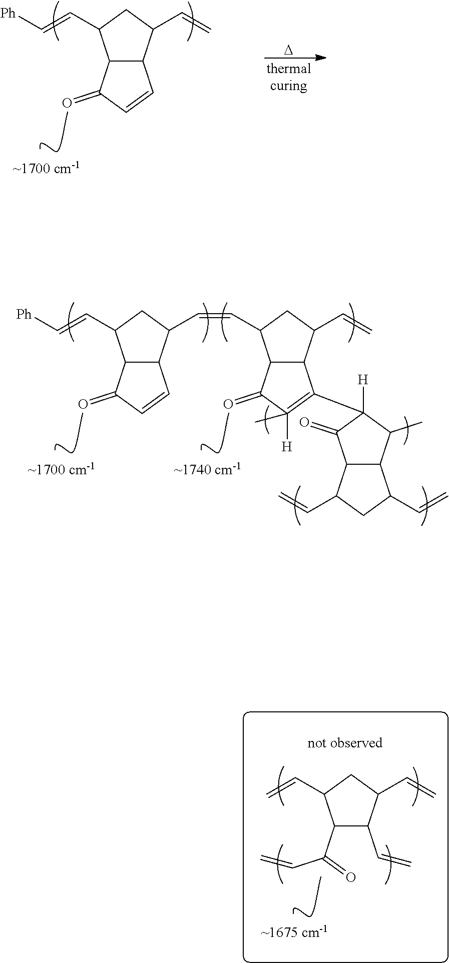

In this example, a polymer embodiment prepared from exo-dicyclopentadienone was cured to provide a crosslinked polymer. Samples of freshly prepared linear poly(exo-dicyclopentadienone) were thermally cured at 110° C. for 8 days. Individual samples were withdrawn at regular intervals and assessed by infrared spectroscopy to determine the extent of crosslinking and the type of chemical crosslink present within the material. Within the first 24 hours, a second carbonyl stretch was observed at higher wavenumbers than the carbonyl stretch for the linear polymer. These data (see FIG. 10) are consistent with a crosslinking mechanism in which the enone motif within the pendent cyclopentenone residues undergo olefin-addition polymerization. The data are inconsistent with the formation of crosslinks through secondary metathesis events.

### Example 11

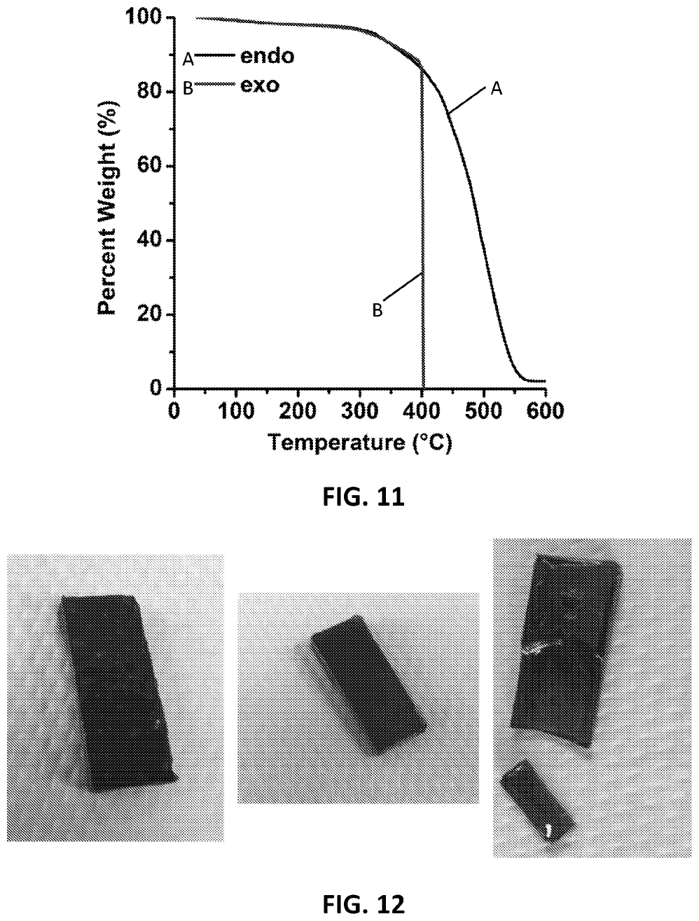

In this example, the thermal stability of two different polymer embodiments (poly(exo-dicyclopentadienone) and oxaPDCPD) was evaluated using thermogravimetric analysis (TGA). Samples were heated in a Shimadzu TGA-50 instrument to 900° C., using a ramp rate of 5° C. The data (see FIG. 11) indicate that both types of functionalized polymers are stable to approximately 400° C.

### Example 12

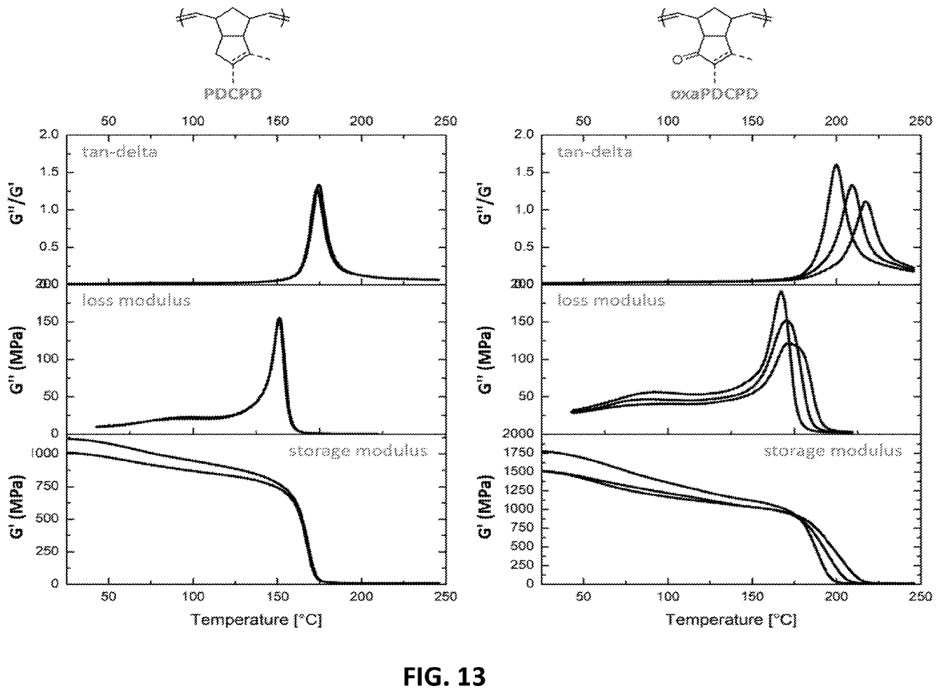

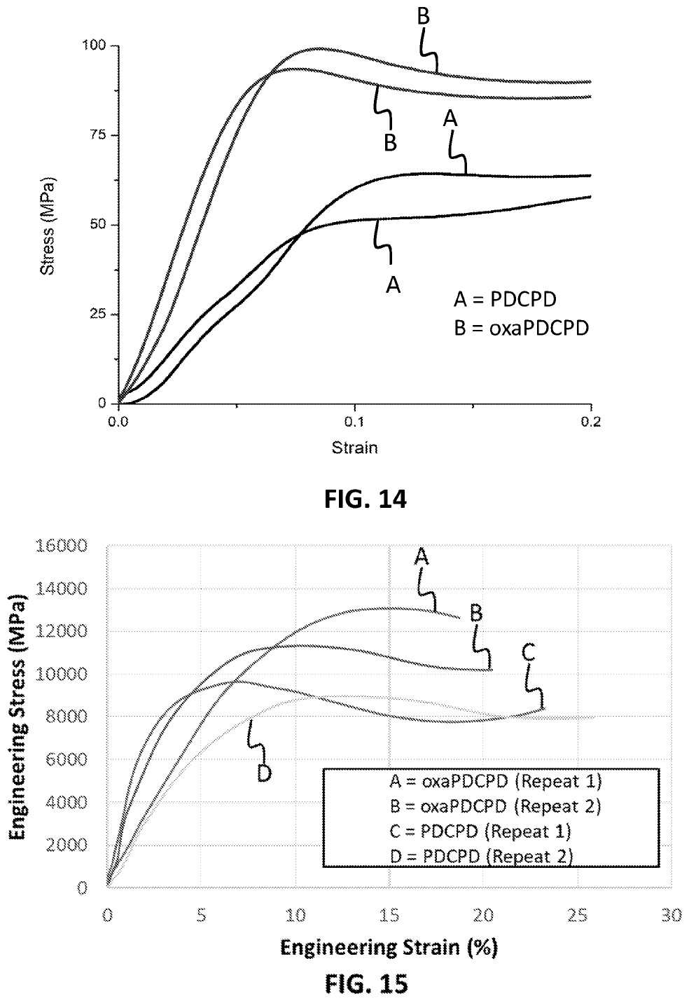

In this example, objects made of a polymer embodiment, namely oxaPDCPD, were obtained using reaction-injection molding (RIM). endo-Dicyclopentadienone (5.568 g; 38.12 mmol) was melted and tri-n-butyl phosphite (50.2 mg; 0.201 mmol) was added. Then Grubbs 2nd generation catalyst (162.4 mg; 0.1912 mmol; 0.5 mol %) was added, and the mixture was heated at 60° C. and sonicated until homogenous. The solution was taken up by syringe, injected into an aluminum mold, and cured at 110° C. for 40 minutes. The solid samples were extracted from the mold using a CNC machined punch plate. Images of the objects made with this method are shown by FIG. 12. For the right-most photographic image in FIG. 12, the object was broken to facilitate examining the internal structure of the object. FIG. 13 shows the dynamic mechanical analysis (DMA) data of the reaction injection molded oxaPDCPD as compared with that of polydicyclopentadiene (PDCPD). As can be seen in FIG. 13, oxaPDCPD exhibits a significantly higher storage modulus, combined with a significantly enhanced glass transition temperature. FIG. 14 shows the stress-strain curves of low strain rate compression strength for oxaPDCPD as compared with PDCPD. An Instron 5969 dual column testing instrument was used for low strain rate compression evaluations. FIG. 15 shows stress-strain curves of high strain rate compression strength for oxaPDCPD as compared with PDCPD. A split-Hopkinson pressure bar system was used to evaluate high strain rate compression strength using a striker diameter of 1.5″ and a testing pressure of 59 Pa.

### Example 13

In this example, injection molding of a copolymer of ethylidene norbornene and PDCPD (0.05/0.95 ENB/PDCPD) was performed. DCPD was melted at 30° C. and 5% by weight ethylidene norbornene (ENB) was added to obtain a liquid. tri-n-butyl phosphite inhibitor (2 eq vs Grubbs 2nd generation catalyst) was mixed through and Grubbs 2nd generation catalyst was then added. The mixture was sonicated to obtain a homogenous resin. The resin was injected with a plastic syringe into an aluminum mold. Alternatively, a light vacuum was applied to the aluminum mold and resin was pulled through the mold. The mold was transferred to a 110° C. oven and allowed to cure for 40 minutes.

### Example 14

In this example, injection molding of oxaPDCPD was performed. oxaDCPD was melted at 60° C. and combined with tri-n-butyl phosphite inhibitor (2 eq vs Grubbs 2nd generation catalyst). Grubbs 2nd generation catalyst was then added and the mixture was sonicated in a warm bath to obtain a homogenous resin. The resin was injected with a plastic syringe into an aluminum mold preheated to ˜45° C. The mold was transferred to a 110° C. oven and allowed to cure for 40 minutes.

Reaction Injected Poly-endo-dicyclopentadienone characterization information: Physical State: semi translucent dark brown, hard plastic; Density: 1.06 g/cm³; Tensile Strength (measured using ATM type V dogbones and measured according to ASTM D638): 53.5+/−2.3 MPa; Strain at Break (measured by evaluating the deformation to the initial length of the specimen): 4.6+/−1.2%; Young's Modulus (obtained from the linear portion of the corresponding stress/strain curve of the tensile plot): 1.47+/−0.14 GPa; Storage Modulus (measured on 4.75×8×20 mm samples): 1601+/−152 MPa; Vicker's Hardness: 16.6+/−1.1; Compression Strength: 100.8+/−14.6 MPa; Glass Transition Temperature (DMTA): 208.6+/−8.6° C.; and Glass Transition Temperature (DSC): 200° C.

Comparative reaction injected Poly-endo-dicyclopentadiene characterization information: Physical State: translucent yellow, hard plastic; Tensile Strength (measured using ATM type V dogbones and measured according to ASTM D638): 39.8+/−1.8 MPa; Strain at Break (measured by evaluating the deformation to the initial length of the specimen): 7.2+/−1.7%; Young's Modulus (obtained from the linear portion of the corresponding stress/strain curve of the tensile plot): 1.22+/−0.02 GPa; Storage Modulus (measured on 4.75×8×20 mm samples): 1117.1+/−75.0 MPa; Vicker's Hardness: 13.9+/−0.6; Compression strength: 55.4+/−5.9 MPa; and Glass Transition Temperature (DMTA): 173.9+/−0.5.

### Example 15

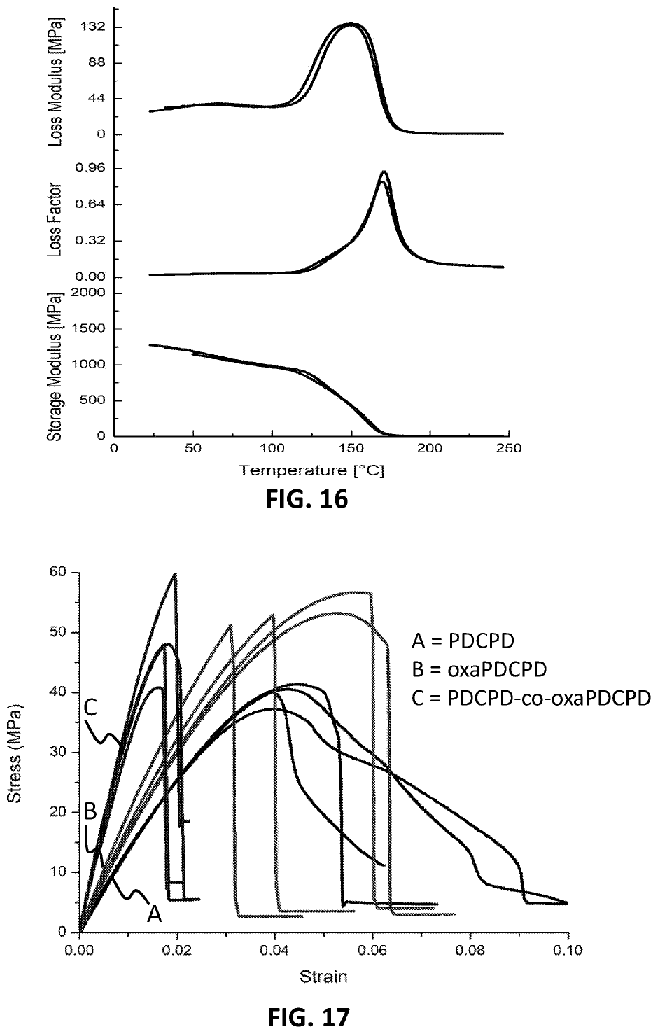

In this example, injection molding of a copolymer of DCPD and oxaDCPD (0.5/0.5 DCPD/oxaDCPD) was performed. A 1:1 ratio of oxaDCPD and DCPD by weight were melted together at 60° C. Tri-n-butyl phosphite inhibitor (2 eq vs Grubbs 2nd generation catalyst) was added and mixed throughout. Grubbs 2nd generation catalyst was then added and the mixture was sonicated in a warm bath to obtain a homogenous resin. The resin was injected with a plastic syringe into an aluminum mold preheated to −45° C. The mold was transferred to a 110° C. oven and allowed to cure for 40 minutes. FIG. 16 shows the DMA for this embodiment and FIG. 17 shows the comparative stress-strain curves of ultimate tensile strength for PDCPD, oxaPDCPD, and PDCPD-co-oxaPDCPD.

Reaction Injected Poly-endo-dicyclopentadienone-co-dicyclopentadiene characterization information: Physical State: translucent brown, hard plastic; Tensile Strength: 49.1+/−7.9; Strain at Break: 1.9+/−0.2; Young's Modulus: 3.62+/−0.27; Storage Modulus: 1255.7+/−29.4; Glass Transition Temperature (DMTA): 170.4+/−1.1. These properties were measured using techniques similar to those discussed for Example 14.

### Example 16

In this example, injection molding of a copolymer comprising ethylidene norbornene, PDCPD, and oxaDCPD (0.05/0.45/0.5 ENB/PDCPD/oxaDCPD) was performed. DCPD was melted at 60° C. and 10% by weight ethylidene norbornene (ENB) was added. oxaDCPD was melted into the resulting liquid to obtain a resin with 45% DCPD, 5% ENB and 50% oxaDCPD by weight. Tri-n-butyl phosphite inhibitor (2 eq vs Grubbs 2nd generation catalyst) was mixed through and Grubbs 2nd generation catalyst was then added. The mixture was sonicated to obtain a homogenous resin. The resin was then injected with a plastic syringe into an aluminum mold. Alternatively, a light vacuum was applied to the aluminum mold and resin was pulled through the mold. The mold was transferred to a 110° C. oven and allowed to cure for 40 minutes.

### Example 17

In this example, ballistic testing with 17 grain fragment simulating projectiles was conducted on both PDCPD and oxaPDCPD plaques produced by reaction injection molding. Testing revealed that 10 mm PDCPD plaques were effective in testing with projectile velocities of ca. 300 m/s. At this testing range, reproducible velocity for the projectile was achieved, while still achieving measurable velocity reduction for each projectile. While a few partial penetrations were observed, most shots penetrated the material, with an average velocity reduction of 74% (Table 1).

Four 10 mm PDCPD plaques were tested in order to assess the material variability. Excellent consistency between samples was observed, and each plaque consistently showed a velocity reduction across three to five shots of 74%.

TABLE 1

| | | Pre-target | Post-target | | Calibrated | |
|---|---|---|---|---|---|---|
| | Powder | Velocity | Velocity | Uncalibrated | Post-Target | Calibrated |
| | load^{(a)} | Radar^{(b)} | Camera^{(c)} | Velocity | Velocity | Velocity |
| Type | (grains) | (m/s) | (m/s) | Reduction (%) | (m/s) | Reduction (%) |
| calibration | 1.5 | 256.6 | 285.3 | −11.2% | 256.4 | 0.1% |
| calibration | 1.5 | 305.4 | 337.2 | −10.4% | 303.0 | 0.8% |
| calibration | 1.5 | 317.6 | 354.4 | −11.6% | 318.5 | −0.3% |
| calibration | 4 | 511 | 567.7 | −11.1% | 510.1 | 0.2% |
| calibration | 6 | 620.2 | 694.5 | −12.0% | 624.1 | −0.6% |
| calibration | 9 | 815 | 908.2 | −11.4% | 816.1 | −0.1% |

| | | |
|---|---|---|
| average calibration = | −11.3% ± 0.5% | 0.0% ± 0.5% |

^{(a)}Trailboss powder used for all shots.
^{(b)}Pre-target radar measurement within 50 cm of the sample.
^{(c)}Post-target camera reading ca. 30 cm behind the sample.

With reproducible performance observed for the polydicyclopentadiene plaques, the ballistic performance of the oxaPDCPD-co-PDCPD copolymer was evaluated. Improved performance in both a 10 mm plaque of PDCPD and a 10 mm puck-shaped sample that was tested at the same time (Table 2) was observed. A larger ratio of partial penetrations was observed (i.e. capture of the projectile within the polymer matrix) and the overall velocity reduction was markedly greater. In fact, a half-thickness oxaPDCPD-co-PDCPD copolymer plaque (5 mm instead of 10 mm in thickness) performed almost as well as the 10 mm PDCPD plaques, with an average velocity reduction of 68% across three projectiles.

TABLE 2

| | Pre-target | Post-target | | Calibrated | Average |
|---|---|---|---|---|---|
| | Velocity | Velocity | | Velocity | Reduction |
| | Radar^{(b)} | Camera^{(c)} | | Reduction | Across |
| Sample | (m/s) | (m/s) | Notes | (%) | Plaque |
| DCPD | 268.2 | 0 | Partial Penetration^{(e)} | 100% | DCPD 76% |
| (10 mm plaque) | 307.2 | 96.2 | | 68.7% | |
| | 327.5 | 135.3 | | 58.7% | |
| DCPD | 314.9 | 105.2 | | 66.6% | DCPD 74% |
| (10 mm plaque) | 314.0 | 79.9 | | 74.6% | |
| | 333.1 | 118.7 | | 64.4% | |
| | 293.3 | 32.6 | | 88.9% | |
| Copolymer | 289.5 | n.d.^{(d)} | No video available^{(d)} | n.d.^{(d)} | Copolymer 91% |
| (10 mm plaque) | 322.0 | 0 | Partial Penetration^{(e)} | 100% | |
| | 322.8 | 89.1 | | 72.4% | |
| | 265.5 | 0 | Partial Penetration^{(e)} | 100% | |
| Copolymer | 307.8 | ca. 1 | Just barely through; | >99% | Copolymer >99% |
| (10 mm puck) | | | caught on mounting tape. | | |
| Copolymer | 291.6 | 124.1 | | 57.4% | Copolymer 68% |
| (5 mm plaque) | 278.5 | 146.9 | | 47.2% | (half-thickness) |
| | 262.9 | 0 | Partial Penetration^{(e)} | 100% | |
| DCPD | 268.5 | 0 | Defects visible in sample. | 100% | DCPD 71% |
| (10 mm plaque) | 302.5 | 156.8 | | 48.2% | |
| | 274.6 | n.d.^{(d)} | No video available^{(d)} | n.d.^{(d)} | |
| | 310.1 | 102.1 | | 67.1% | |
| | 316.0 | 99.3 | | 68.6% | |
| DCPD | 306.2 | 97.1 | | 68.3% | DCPD 75% |
| (10 mm plaque) | 300.1 | 92.8 | | 69.1% | |
| | 300.7 | 85.7 | | 71.5% | |
| | 316.3 | 111.2 | | 64.8% | |
| | 267.0 | 0 | Partial penetration^{(e)} | 100% | |

^{(a)}All shots were made using 1.5 grains of Trailboss powder.
^{(b)}Pre-target radar measurement within 50 cm of the sample.
^{(c)}Calibrated post-target camera reading ca. 30 cm behind the sample.
^{(d)}No data due to lost projectile (e.g. due to rebound from sample).
^{(e)}Partial penetrations resulted in the projectile being embedded in the sample.

The energy transferred to the panel can be assessed by measuring the initial velocity and the residual velocity. The difference between the energy transferred to the target and the kinetic energy of the bullet gives the absorption of the energy by the panel:

$${E}_{t} = \frac{1}{2} {m}_{p} ( {ν}_{i}^{2} - {v}_{r}^{2} ) and {E}_{c} = \frac{1}{2} {m}_{p} {v}_{i}^{2} gives Δ {E}_{f} = \frac{{E}_{t}}{{E}_{c}} \times 100$$

The oxaPDCPD-co-PDCPD polymer was determined as being capable of absorbing a greater degree of energy than the PDCPD parent material.

Examination of the samples after ballistic testing confirmed similar mechanical relationships between PDCPD and oxaPDCPD to that of the mechanical testing. The PDCPD samples that were shot had mostly clean holes where the projectile had passed through the object (ejecting a plug of polymer in the process) without significantly damaging the surrounding material. By contrast, in some embodiments, the oxaPDCPD-co-PDCPD samples were better at stopping projectiles, but tended to suffer more brittle fracture in the process. Despite this, the projectile itself was nearly stopped in its path; it possessed just enough energy to exit the puck, where it was caught by a thin piece of packing tape that had been used to mount the irregularly shaped test sample.

Similar observations were made through high-speed videos that were recorded for representative PDCPD and copolymer samples. In a video recorded of a projectile passing through a PDCPD sample, one can see a clean, unimpeded passage of the projectile, together with ejection of a neat plug of PDCPD. By contrast, in three subsequent videos recorded of the oxaPDCPD-co-PDCPD copolymer, one can see one complete stoppage (in which the crosslinked polymer sample exhibits an impressive degree of flex to capture the projectile), one complete penetration, and one shot that came slowly tumbling out the back of the test sample accompanied by a ‘puzzle piece’ of polymer that clearly resulted from brittle fracture. At the same time, the energy that went into fracturing the material appears to have slowed down the projectile.

### Example 18

In this example, finite-element ballistic modeling was performed, based upon the mechanical data disclosed herein. These data allowed for the creation of a robust theory of material for PDCPD and oxaPDCPD, which in turn allowed models of ballistic performance to be constructed in silico.

These initial models predicted an enhanced ballistic performance for oxaPDCPD relative to PDCPD. Finite element modeling was carried out using a simulated 85×85 mm plaque, varying the thickness between 5 to 20 mm.

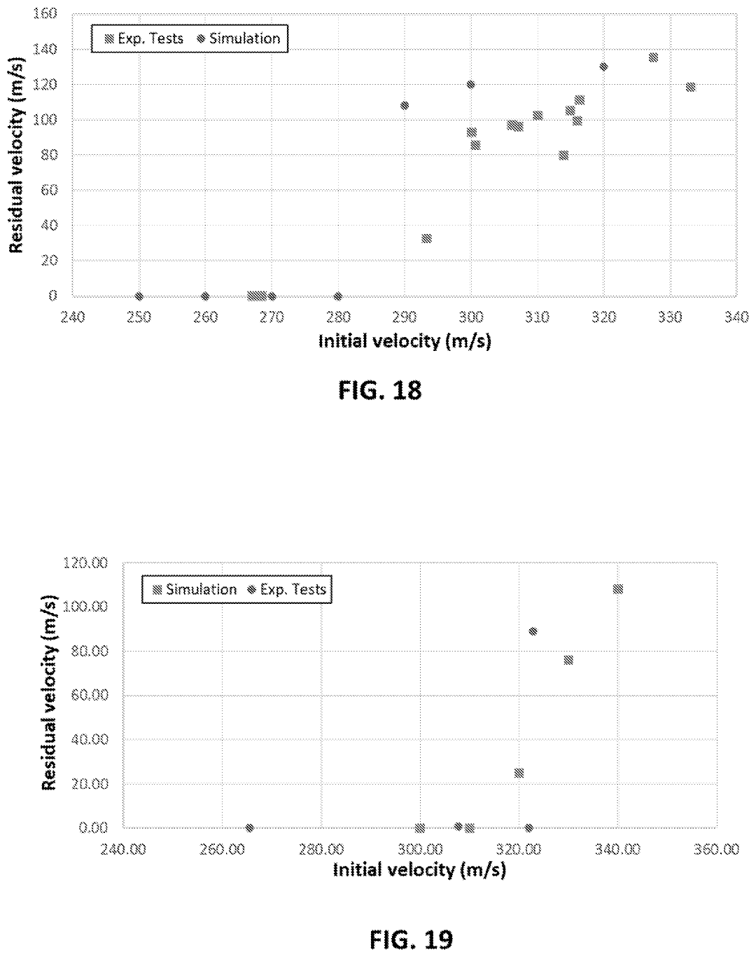

One shot was modeled per simulated sample, using a 1.102 g projectile of AISI steel, with a calibre of 5.6 mm (0.22 cal). The length of the projectile was 6.1 mm and the radius was 2.74 mm. Good agreement was found between theory and experiment (FIGS. 18 and 19, which show results for PDCPD and oxaPDCPD-co-PDCPD, respectively). The V50 was estimated at 285 m/s for the simulation, whereas the V50 in the actual tests was approximately 282 m/s. Modeling using the oxaPDCPD-co-PDCPD copolymer revealed similar trends, except that the ballistic response was shifted to higher input energies. Here again, the experimental tests and simulation are in very good agreement.

The validated ballistic model was then used to predict the V50 for different thicknesses of PDCDP and oxaPDCPD-co-PDCPD. As illustrated in Table 3 an increase of up to 18% is predicted upon switching from the parent polymer to the copolymer.

TABLE 3

| | plate thickness | | |
|---|---|---|---|
| | 5 mm | 10 mm | 15 mm |
| PDCPD | V50 = 255 m/s | V50 = 285 m/s | V50 = 330 m/s |
| oxaPDCPD-co- | V50 = 280 m/s | V50 = 320 m/s | V50 = 390 m/s |
| PDCPD | | | |
| relative increase | 10% | 12% | 18% |

And, the predicted performance of PDCPD and oxaPDCPD from the finite element simulations were compared with the performance of a representative grade of aluminum used in ballistic protection (Table 4).

TABLE 4

| | Thickness | Areal density | V50 | |
|---|---|---|---|---|
| Material | (mm) | (kg/m²)^{(a)} | (m/s) | V50/AD |
| PDCPD | 5 | 6.25 | 255 | 40.8 |
| PDCPD | 10 | 12.5 | 285 | 22.8 |
| PDCPD | 15 | 18.75 | 330 | 17.6 |
| Co-PDCPD | 5 | 6.25 | 280 | 44.8 |
| Co-PDCPD | 10 | 12.5 | 320 | 25.6 |
| Co-PDCPD | 15 | 18.75 | 390 | 20.8 |
| Alu 5083 | 5 | 13.75 | 398 | 28.9 |
| Alu 5083 | 10 | 27.5 | 742 | 26.9 |
| Alu 5083 | 15 | 41.25 | 1044 | 25.3 |

^{(a)}using a conservatively assumed density of 1.25 g/cm³ for both PDCPD and oxaPDCPD.

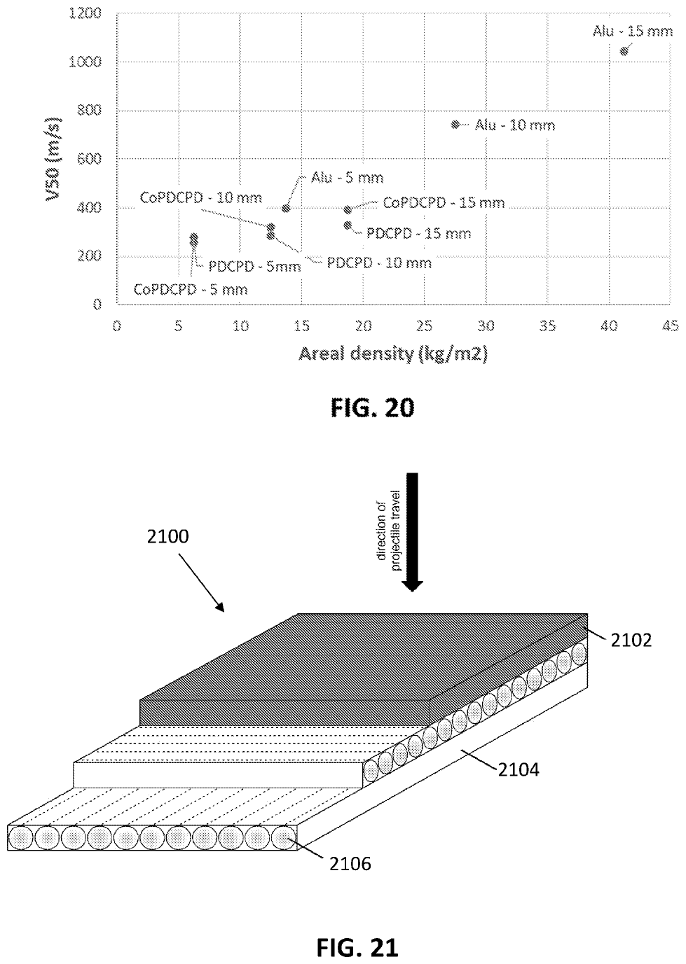

A good ballistic material can have a high V50 for a low areal density. A comparison of the performance of aluminum 5083 to PDCPD and oxaPDCPD-co-PDCPD on this basis is provided by see FIG. 20. This comparison, however, utilizes the polymer materials without any fiber-reinforcing agents. The properties of the polymer materials should improve considerably when fiber-reinforcement is added. The oxaPDCPD-co-PDCPD copolymer is particularly impressive in that it already has good ballistic performance and will have improved interlaminar adhesion with various commercially available fiber-reinforcing agents.

### Example 19

In this example, both ceramic-oxaPDCPD composites for use as appliqué armor for deployment in Light Utility Vehicles, and fiber-reinforced oxaPDCD composites for use as structural armor components are described. In both systems, the oxaPDCPD matrix is compared with both the parent PDCPD polymer and with the oxaPDCPD-co-PDCPD copolymer. Also, “composite-of-composite” systems, wherein a ceramic strikeface fronts an oxaPDCPD (or oxaPDCPD-co-PDCPD) energy/fragment-absorbing layer that is strengthened with fiber reinforcement (FIG. 21), are evaluated. As FIG. 21 shows, the composite material 2100 can comprise strikeface 2102 (which can be made of a ceramic and/or steel material) that is responsible for breaking up an incoming projectile into high-energy fragments that are then stopped by polymer layer 2104 (e.g., a layer comprising an oxaPDCPD and/or an oxaPDCPD-co-PDCPD co-polymer backing layer. In some embodiments, reinforcing fiber layer 2106 can be included, which can be made of carbon, glass, or UHMWPE fiber.

The performance of representative fiber-reinforced composites (using glass, carbon, and UHMWPE fibers) in tensile and 3-point bending tests is evaluated. Single fiber pull-out experiments are used to report upon interfacial adhesion. Specific comparisons with traditional composite materials (e.g. glass and carbon fiber-reinforced epoxy) also are conducted.

Then, additional studies (using both oxaPDCPD on its own, and oxaPDCPD-fiber composites) with larger-diameter FSPs (20 mm) are performed. Then, guided by the modelling data of Example 17, full-fledged ceramic-oxaPDCPD, ceramic-PDCPD, and ceramic-steel composite armor panels (20″×20″; panels will be made with and without fiber reinforcement) are constructed. These are tested in standard ballistic experiments at STANAG Level 1 (e.g. 7.62×51 mm NATO Ball projectiles @ 833 m/s, and 5.56×51 mm NATO Ball projectiles (SS109) @ 910 m/s) and STANAG Level 3 (e.g. 7.62×51 mm AP projectiles @ 930 m/s). oxaPDCPD samples also are evaluated in blast protection models. And, steel-(oxa)PDPD composites, in which the polymer layer is placed both in front and behind the steel defeat layer, also are evaluated.

In view of the many possible embodiments to which the principles of the present disclosure may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A compound, having a structure according to Formula I 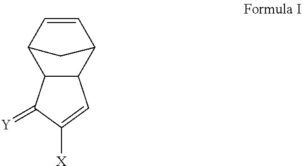
wherein
X is selected from hydrogen, deuterium, halogen, aliphatic, haloaliphatic, haloheteroaliphatic, heteroaliphatic, aromatic, an organic functional group, a detectable moiety, or an active component, or any combination of such groups; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, or an organic functional group; provided that if Y is O, then X is not hydrogen.

2. The compound of claim 1, wherein X is hydrogen, deuterium, Cl, Br, I, alkyl, alkynyl, alkenyl, heteroalkyl, heteroalkenyl, heteroalkynyl, aryl, heteroaryl, hydroxyl, aldehyde, ester, amide, ketone, a fluorophore, an active component, or a combination thereof.

3. The compound of claim 1, wherein X is hydrogen; deuterium; Br; I; lower alkyl; —C≡CR^{d}, wherein R^{d} is selected from hydrogen, aliphatic, haloaliphatic, haloheteroaliphatic, heteroaliphatic, aromatic, or an organic functional group; —C═CR^{d}R^{e}, wherein R^{d} and R^{e} independently are selected from hydrogen, aliphatic, haloaliphatic, haloheteroaliphatic, heteroaliphatic, aromatic, or an organic functional group; thioether; ether; amine; hydroxyl; aldehyde; ester; amide; ketone; an aryl ring system comprising from 6 ring atoms to 10 ring atoms; a heteroaryl ring system comprising from 5 ring atoms to 10 ring atoms, with at least one heteroatom; fluorescein; rhodamine; BODIPY; an antibacterial agent; an antifungal agent; an anticancer agent; a peptide promoting cellular adhesion; a signaling factor controlling cellular growth or motility; a vitamin; a cofactor; a fire retardant; a pH sensor; an H₂S sensor; an antibody-based sensor; or any combination thereof.

4. The compound of claim 1, wherein Y is O; S; N-aromatic; or N—OR^{d} or N—NR^{d}R^{e}, wherein R^{d} and R^{e} independently are selected from hydrogen, alkyl, heteroalkyl, heteroalkenyl, heteroalkynyl, aryl, or heteroaryl.

5. The compound of claim 1, wherein Y is O, S, Se, N-phenyl, N-pyridinyl, N—OH, N—OMe, N—OEt, N—NH₂, N—NHMe, N—N(Me)₂, N—N(Et)₂, or N—N(Me)Et.

6. The compound of claim 1, wherein the compound is selected from 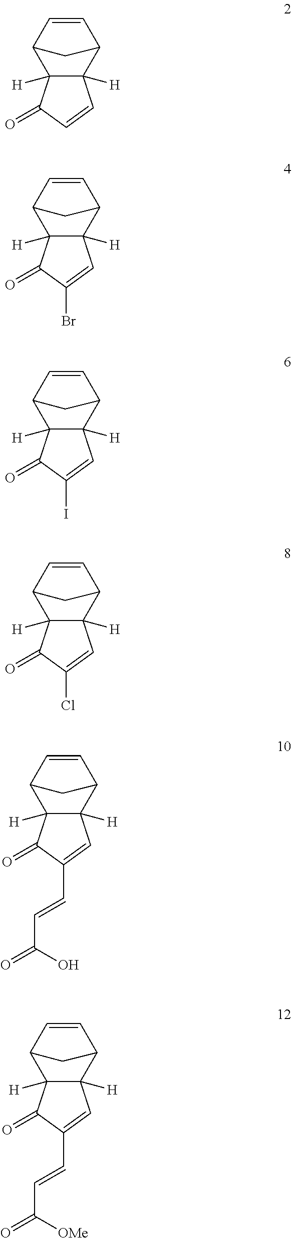 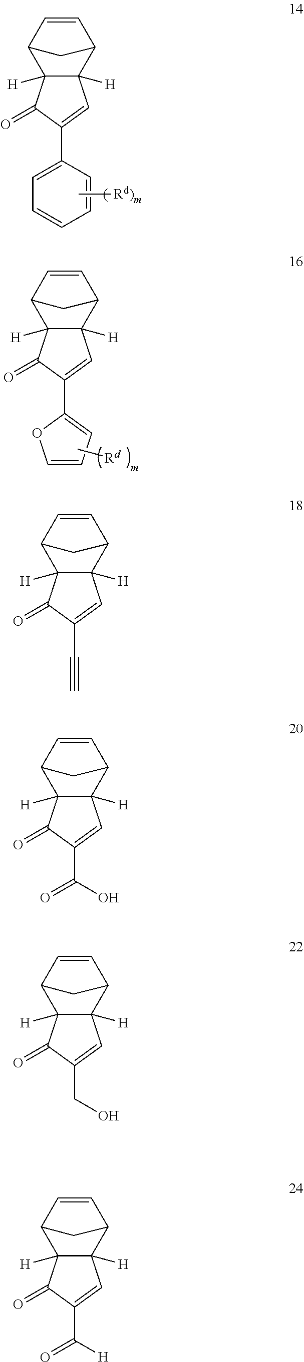 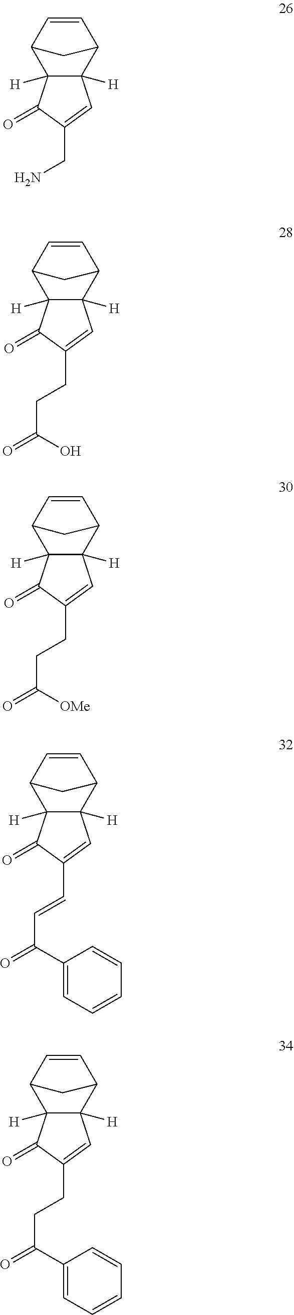 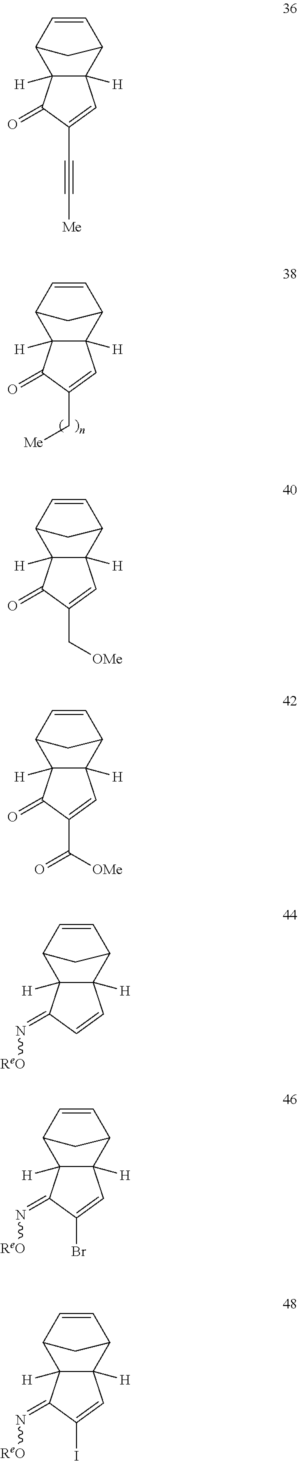 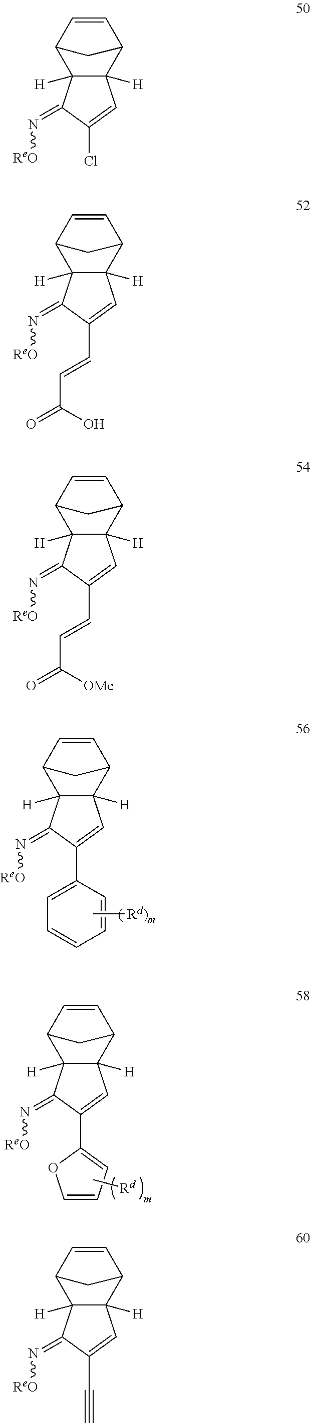 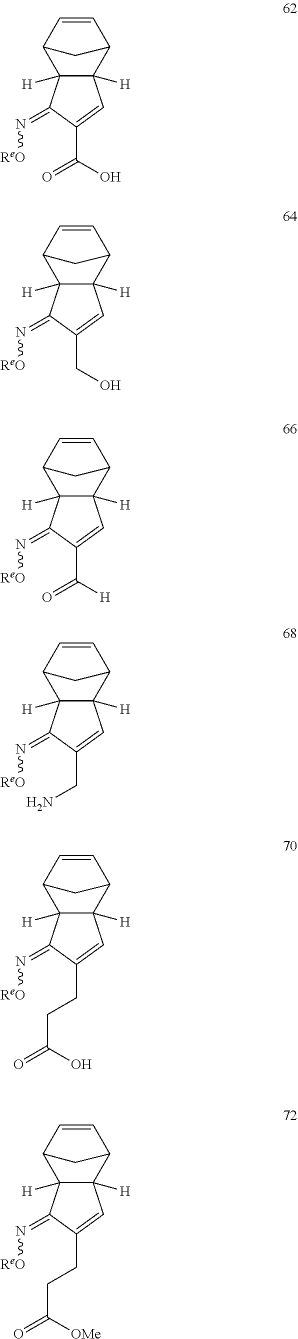 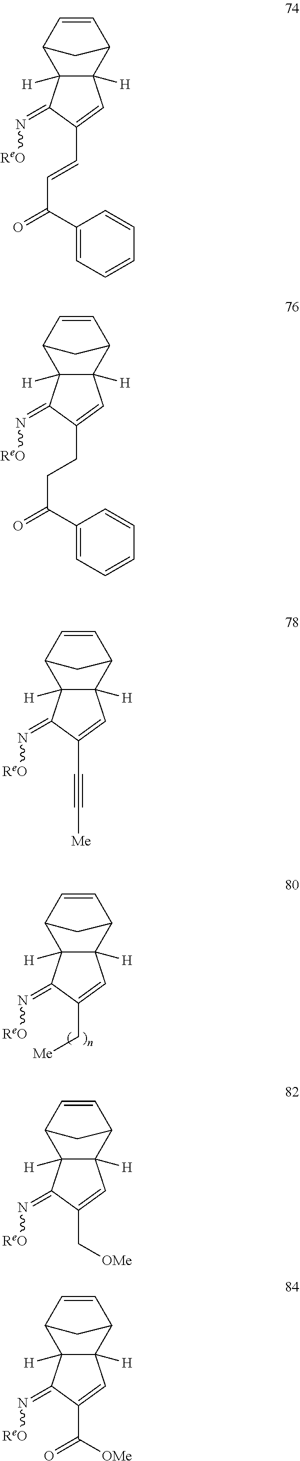 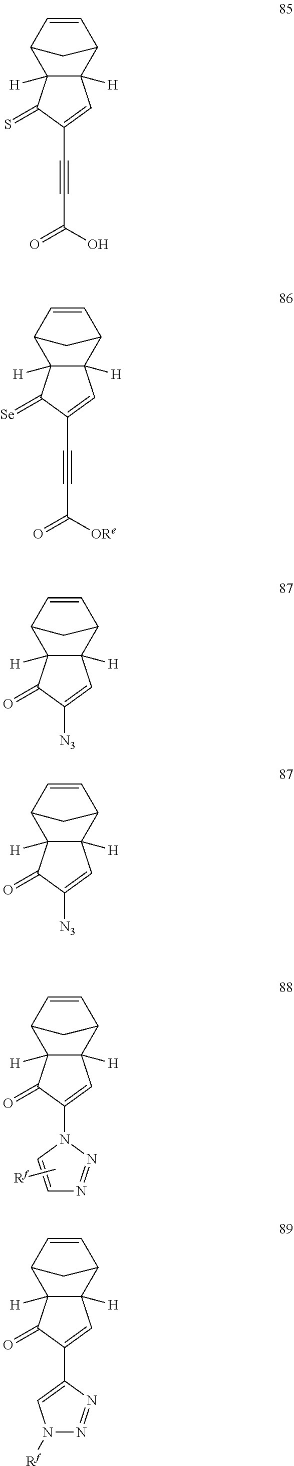
wherein R^{d} and R^{e} are independently selected from hydrogen, aliphatic, haloaliphatic, haloheteroaliphatic, heteroaliphatic, aromatic, or an organic functional group; R^{f} is an active component or a detectable moiety; each n independently is an integer selected from 0 to 25; and each m independently is an integer selected from 0 to 5.

7. The compound of claim 1, wherein the compound is an endo isomer.

8. The compound of claim 1, wherein the compound is an exo isomer.

9. A polymer, comprising at least one monomer subunit having a structure according to Formula IA 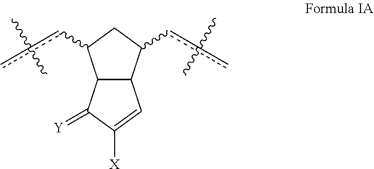
wherein
X is selected from hydrogen, deuterium, halogen, aliphatic, haloaliphatic, haloheteroaliphatic, heteroaliphatic, aromatic, an organic functional group, a detectable moiety, or an active component, or any combination thereof; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, an organic functional group, a detectable moiety, an active component, or a combination thereof.

10. The polymer of claim 9, wherein the polymer is a homopolymer having a structure according to Formula II

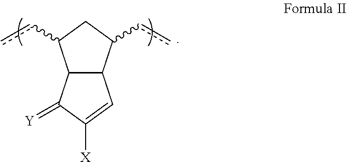

11. The polymer of claim 9, wherein the polymer is a copolymer having a structure according to Formula IIIA or IIIB 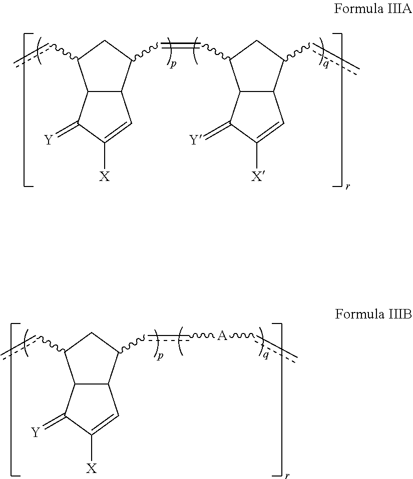
wherein each X and each X′ independently for each occurrence is selected from hydrogen, deuterium, halogen, aliphatic, haloaliphatic, haloheteroaliphatic, heteroaliphatic, aromatic, an organic functional group, a detectable moiety, or an active component, or any combination thereof; each Y and each Y independently for each occurrence is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, an organic functional group a detectable moiety, an active component, or a combination thereof; A is an aliphatic group; each of p, q, and r independently is an integer of at least 1; and provided that either (i) X and X′ are different, or (ii) Y and Y′ are different.

12. The polymer of claim 11, wherein A is an acyclic aliphatic group or a cyclic aliphatic group.

13. The polymer of claim 9, wherein the polymer is crosslinked with a second polymer.

14. The polymer of claim 9, wherein the polymer comprises an end-capping group selected from hydrogen, aromatic, aliphatic, haloaliphatic, haloheteroaliphatic, heteroaliphatic, or an organic functional group.

15. A method, comprising:
forming a polymer by combining (i) a catalyst comprising ruthenium (Ru), molybdenum (Mo), tungsten (W), titanium (Ti), tantalum (Ta), or a combination thereof; and (ii) a compound having a Formula I 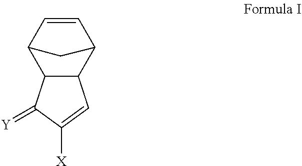
wherein
X is selected from hydrogen, deuterium, halogen, aliphatic, haloaliphatic, haloheteroaliphatic, heteroaliphatic, aromatic, an organic functional group, a detectable moiety, or an active component, or any combination of such groups; and
Y is selected from O; S; Se; or NR^{c}, wherein R^{c} is hydrogen, —OR^{d}, or —NR^{d}R^{e}, wherein R^{d} and R^{e} independently are selected from hydrogen, aliphatic, haloaliphatic, haloheteroaliphatic, heteroaliphatic, aromatic, or an organic functional group.

16. The method of claim 15, further comprising exposing the polymer to conditions sufficient to promote crosslinking.

17. The method of claim 16, wherein the conditions sufficient to promote crosslinking include exposing the polymer to air, radical crosslinking conditions, anionic crosslinking conditions, and/or a crosslinking reagent.

18. The method of claim 15, further comprising exposing the polymer to hydrogenation conditions to provide a polymer comprising saturated bonds between monomer subunits.

19. The method of claim 15, further comprising exposing the polymer to conditions capable of transforming a chemical moiety of the polymer to a different chemical moiety.

20. The method of claim 19, wherein the conditions are selected from reductive amination conditions, 4+2 cycloaddition conditions, oxime-forming conditions, saponification conditions, amide bond forming conditions, transesterification conditions, palladium-mediated cross-coupling conditions, click chemistry conditions, conjugate addition conditions, carbonyl-reducing conditions, hydrogenation conditions, or combinations thereof.

21. A composite material, comprising:
a strikeface layer;
a polymer layer that is physically associated with the strikeface layer and that comprises a polymer according to claim 9;
a reinforcing fiber layer that is physically associated with the polymer layer.

22. The composite material of claim 21, wherein the strikeface layer is made of a ceramic or steel material, the polymer layer comprises an oxaPDCPD polymer, or an oxaPDCPD-co-PDCPD co-polymer, and the reinforcing fiber layer is made of a glass, carbon, or ultra-high-molecular-weight polyethylene.
